(19) United States
(12) Patent Application Publication
(10) Pub. No.: US 2006/0199260 A1
(43) Pub. Date: Sep. 7, 2006

(54) Microbioreactor for continuous cell culture

(76) Inventors: Zhiyu Zhang, Cambridge, MA (US); Paolo Boccazzi, Cambridge, MA (US); Hyun-Goo Choi, Malden, MA (US); Klavs F. Jensen, Lexington, MA (US); Anthony J. Sinskey, Boston, MA (US)

Correspondence Address: CHOATE, HALL & STEWART LLP, TWO INTERNATIONAL PLACE, BOSTON, MA, 02110, US

(21) Appl. No.: 11/236,453

(22) Filed: Sep. 26, 2005

Related U.S. Application Data
(63) Continuation-in-part of application No. 10/816,046, filed on Apr. 1, 2004.
Application No. 10/816,046 is a continuation-in-part of application No. 10/427,373, filed on May 1, 2003.
(60) Provisional application No. 60/376,711, filed on May 1, 2002.
(60) Provisional application No. 60/613,140, filed on Sep. 24, 2004.

(51) Int. Cl. C12M 3/00; C12M 1/12

(52) U.S. Cl. 435/293.1; 435/297.2; 435/29

(57) ABSTRACT

The present invention microscale bioreactors (microfermentors) and microscale bioreactor arrays for use in culturing cells. The microfermentors include a vessel for culturing cells and means for providing oxygen to the interior of the vessel at a concentration sufficient to support cell growth, e.g., growth of bacterial cells. Depending on the embodiment, the microfermentor vessel may have various interior volumes of less than approximately 1 ml. The microfermentors may include an aeration membrane and optionally a variety of sensing devices. Methods of using the microfermentors, e.g., to select optimum cell strains or bioprocess parameters are provided. The microbioreactors having a variety of different designs, some of which incorporate active fluid mixing and/or have the capability to operate in batch, fed-batch, or continuous mode. In certain embodiments the microreactors operate as microchemostats. Methods for culturing cells under chemostat conditions in a microbioreactor are also provided.

### CROSS-REFERENCE TO RELATED APPLICATION

This application is continuation-in-part of U.S. patent application Ser. No. 10/816,046, filed Apr. 1, 2004, which is a continuation-in-part of 10/427,373 filed May 1, 2003, which claims priority to U.S. Provisional Patent Application 60/376,711, filed May 1, 2002, all of which are incorporated herein by reference. This patent application claims priority to, and the benefit of, U.S. Provisional Patent Application Ser. No. 60/613,140, filed Sep. 24, 2004, which is incorporated herein by reference.

### BACKGROUND OF THE INVENTION

A critical driving force behind research in bioprocess science and engineering continues to be the demand for fast and accurate analytical information that can be used, for example, to evaluate the interactions between biological systems and bioprocess operations. One significant challenge is to carry out large numbers of experiments rapidly and efficiently. This issue is of particular importance since many of the advances in molecular biology now lead to large numbers of potential biological systems that contain evolved biocatalysts, new pathway designs, and a variety of unique biological organisms from diverse sources.

Bioprocess development techniques have been unable to keep pace with the current rate of discovery and genetic manipulation in biological systems. Of the hundreds of thousands of genetic and process permutations that can now be designed, only a small fraction can be tested using standard bioprocess practices. Bench-scale bioreactors, with typical volumes of between 2 and 10 liters, are limiting for a number of reasons including the time required to obtain sufficient data for a biological system, the effort required to obtain the data, and the high cost of these systems. Currently the smallest bioreactors that are available commercially have working volumes of approximately 0.5 liters (Sixfors, Appropriate Technical Resources) and allow six parallel fermentations to be carried out.

There exists a need for systems that allow rapid testing, process development, and optimization to be carried out through parallel fermentations. In particular, there exists a need for microscale bioreactor systems that allow multiple experiments to be performed in parallel without an accompanying increase in cost. In addition, there exists a need for microscale bioreactor systems wherein experimental conditions and results obtained in the microscale bioreactor may be translated into predictable large-scale bioprocess operations.

The above needs are not limited to bioprocess development but extend more generally to other settings, e.g., any settings in which it is desired to test or optimize reaction conditions, substrates, etc.

### SUMMARY OF THE INVENTION

The present invention encompasses the recognition that the ability to perform cell culture, e.g., for testing, strain optimization, bioprocess parameter optimization, etc., in bioreactors with small volumes offers significant advantages as compared with fermentations performed in traditional production scale or bench scale fermentors. Accordingly, the invention provides a variety of microscale bioreactors (microfermentors), microscale bioreactor arrays, and associated apparatus as well as methods for use thereof. The invention further encompasses the recognition that the use of small scale reactors in process development and optimization extends beyond the field of bioproduction. The testing and/or optimization of any type of chemical or biochemical reaction would benefit from the availability of small-scale reactors that could be operated in parallel. Thus any of the bioreactors, bioreactor arrays, and reactor operation units described herein may be used for chemical process development and/or optimization.

In one aspect, the invention provides a microscale bioreactor (microfermentor) comprising a vessel having an interior volume of less than 200 microliters and means for providing oxygen to the vessel at a concentration sufficient to support cell growth. Optionally, the microfermentor includes at least one channel extending from and in communication with the vessel and/or means for introducing a component into the vessel or removing a sample from the vessel via a channel. According to certain embodiments of the invention the means for providing oxygen comprises an aeration membrane, wherein oxygen diffuses through the membrane into the vessel. The membrane may comprise, for example, a fluoropolymer or a silicone.

In another aspect, the invention provides microscale bioreactors as described above and having means for quantification of biomass, e.g., by measuring the optical density of the culture medium, by measuring the concentration of a cell metabolite, etc. Optionally, the microscale bioreactors may include means for measuring dissolved oxygen within the culture vessel, and/or means for measuring at least one other parameter, which may be, e.g., temperature, pH, carbon dioxide concentration, carbon source concentration, concentration of an ionic species, and concentration of a cellular metabolite.

According to certain embodiments of the invention the means for measuring biomass and/or a bioprocess parameter comprises an optical sensor, e.g., an optical chemical sensor. In certain embodiments of the invention a waveguide sensor is used. According to certain embodiments of the invention Raman spectroscopy is used to measure one or more bioprocess parameters, e.g., concentrations of various organic compounds present in the medium.

In certain aspects of the invention the microscale bioreactors include means for controlling the temperature and/or pH in the culture vessel. The microscale bioreactor systems of the invention may also include means for delivering nutrients and/or for removing a cell product from the culture vessel.

In another aspect, the invention provides two-vessel microscale bioreactors that comprise a first vessel having an interior volume of 1 ml or less for culturing cells and a second vessel separated from the first vessel at least in part by a membrane permeable to oxygen and carbon dioxide. In certain embodiments of the invention the membrane is permeable to cell products and/or nutrients but not permeable to cells. These microscale bioreactor systems may further include means for flowing a liquid or gas through the second vessel.

In another aspect, the invention provides a microreactor comprising: (a) a first body layer that defines a vessel having an interior volume of less than 1 milliliter; (b) a second body layer that defines a headspace located opposite the vessel; and (c) a gas-permeable membrane that separates the vessel interior from the second body layer. In certain embodiments of the invention the microreactor incorporates a miniature mixing stirbar. In certain embodiments of the invention the microreactor operates either in batch or fed-batch mode.

The invenntion further provides microbioreactors that can be operated as microchemostats and methods of use thereof. For example, the invention provides a microbioreactor comprising comprising: (a) at least one culture vessel having an interior volume of less than 1 ml; (b) a mechanism for continuously mixing the contents of the culture vessel; (c) an inflow port to allow fresh culture medium to be continuously supplied to the culture vessel; and (d) an outflow port to allow culture medium to be continuously removed from the culture vessel at the same rate as fresh medium is supplied, such that a constant fluid volume and constant growth conditions are maintained within the culture vessel for a prolonged period of time after cells cultured in the culture vessel reach a steady state. Preferably the constant growth conditions include constant dissolved oxygen concentration, constant biomass concentration and/or cell density, and constant pH.

The microchemostat can further comprise one or more inflow or outflow channels in communication with the interior of the culture vessel and can comprise a collection chamber. In certain embodiments of the invention the interior of the culture vessel comprises a well located in a first body layer of material and a gas-permeable membrance covering the open portion of the well. Additional layers can serve as gaskets and/or provide structural support and protection. In certain embodiments of the invention means are provided for inhibiting cell growth and/or movement and/or metabolism.

The invention provides a number of methods for using a continuous flow microbioreactor including, (a) introducing at least one cell into a microbioreactor that comprises a culture vessel having an interior volume of less than 1 ml; (b) continuously flowing fresh culture medium into the vessel while continuously removing culture medium containing cells from the culture vessel at the same rate as that which which fresh medium enters the vessel so that a constant medium volume is maintained in the culture vessel; (c) actively mixing the contents of the culture vessel; (d) maintaining the cells for sufficient time to achieve a first steady state. The method may further include maintaining the cells for a period of time and/or collecting a sample and performing an analytical procedure on cells or medium in the sample.

The invention further provides methods for modifying polymeric surfaces with PEG-containing polymers to reduce cell and/or protein adhesion and provides articles, including microbioreactors, comprising PEG-modified surface(s).

In another aspect, the invention provides a chamber sufficiently large to accommodate the microscale bioreactor or microscale bioreactor array, wherein the chamber provides means to control at least one environmental parameter such as temperature or humidity.

The invention further provides bioreactor assemblies (microfermentor arrays) for performing multiple fermentations in parallel. Such assemblies include a plurality of microscale bioreactors as described herein.

In other aspects, the invention includes a variety of methods for using the microscale bioreactors and microscale bioreactor arrays. For example, the invention provides a method of selecting a strain that produces a desired product or degrades an unwanted compound comprising steps of (a) culturing a plurality of different strains, each in an individual microscale bioreactor; (b) measuring the amount of the desired or unwanted product in each of the microscale bioreactors; and (c) selecting a strain that produces an optimum amount of a desired product or degrades a maximum amount of the unwanted compound. The invention further provides a method of selecting a bioprocess parameter comprising steps of (a) culturing an organism type in a plurality of microscale bioreactors, wherein the microscale bioreactors are operated under conditions in which the value of the bioprocess parameter varies and wherein the organism produces a product or degrades a compound; (c) monitoring biomass in each of the microscale bioreactors; and (d) identifying the value of the bioprocess parameter that results in optimum biomass, optimum product formation, or optimum compound degradation. In addition to biomass, other bioprocess parameters may also be monitored, and multiple parameters may be varied. According to certain embodiments of the invention the bioprocess parameter or parameters are actively controlled. The above methods can conveniently be practiced with the apparatus for parallel operation of a plurality of microreactors provided herein.

In another aspect, the invention provides a method of monitoring gene expression comprising: (a) culturing cells in a microbioreactor, wherein the microbioreactor comprises a vessel with an interior volume of 200 μl or less and means for providing oxygen to the interior of the vessel; (b) harvesting some or all of the cells; (c) contacting RNA obtained from the cells, or a nucleic acid transcription product of such nucleic acid, with a microarray comprising probes for a plurality of genes under conditions such that hybridization occurs; and (d) collecting a signal from the microarray, wherein the signal is indicative of the expression level of at least one gene.

The contents of all papers, books, patents, etc., mentioned in this application are incorporated herein by reference. In the event of a conflict or inconsistency between any of the incorporated references and the instant specification or the understanding of one or ordinary skill in the art, the specification shall control, it being understood that the determination of whether a conflict or inconsistency exists is within the discretion of the inventors and can be made at any time.

### BRIEF DESCRIPTION OF THE DRAWINGS

FIGS. 1A and 1B show top and side views of the design of one embodiment of a microfermentor of the invention.

FIG. 2A shows a side view of an embodiment of a two vessel microfermentor in which the fermentation vessel is in contact with the external environment.

FIG. 2B shows a side view of an embodiment of a two vessel microfermentor in which the fermentation vessel is enclosed.

FIG. 3 (upper portion) shows a design of an embodiment of a microfermentor in which components are provided externally to the microfermentor vessel. FIG. 3 (lower portion) shows a schematic of a microfermentor array of the microfermentors depicted in the upper portion of the figure.

FIG. 4A shows a schematic of a platform for an integrated microfermentor array and associated system components.

FIG. 4B shows a schematic of a platform for a microfermentor array and associated microfluidics in which bioprocess parameters are varied among the individual microfermentors.

FIG. 4C shows a schematic of robotic loading and sampling of a microfermentor array.

FIG. 5 shows a schematic illustration of the formation of an oligo(ethylene oxide) self-assembled monolayer on a metal oxide surface.

FIG. 6 shows a strategy for generating a self-assembled film incorporating a recognition element.

FIG. 7 shows a schematic illustration of a surface-initiated ring-opening metathesis polymerization from a hydrated metal oxide surface.

FIG. 8 shows schematics of straight (top) and serpentine (bottom) waveguides.

FIG. 9 shows an example of a microfabricated heat exchanger.

FIG. 10 is a flowchart of the fabrication procedure employed in one embodiment of the invention.

FIG. 11 shows a top view of a completed microfermentor fabricated as outlined in FIG. 10 and filled with phenol red.

FIG. 12 illustrates a one-dimensional resistance-in-series model of the membrane and the medium, which was used to model oxygen diffusion into a microfermentor.

FIG. 13A shows the calculated steady state oxygen concentration using a one-dimensional resistance-in-series model obtained assuming a cell population homogenously spread throughout the medium.

FIG. 13B shows the calculated steady state oxygen concentration profile using a one-dimensional resistance-in-series model of membrane and medium obtained assuming a membrane thickness of 100 μm, a microfermentor depth of 300 μm, and a cell population of 10¹¹ cells/L, with the cells at the bottom of the microfermentor (heterogenous case).

FIG. 14 shows a schematic of a microscale bioreactor system with associated optical excitation and detection sources.

FIGS. 15A and 15B depicts two views of a microfermentor system in which a microfermentor is placed in an environmental control chamber. The transparent glass slide is not readily visible.

FIG. 16 shows optical density and dissolved oxygen data obtained from batch fermentation of E. coli in a microfermentor in medium without glucose.

FIG. 17 shows optical density and dissolved oxygen data obtained from batch fermentation of E. coli in a microfermentor in medium containing 30 g/L glucose.

FIGS. 18A and 18B show optical density and dissolved oxygen data obtained from batch fermentation of E. coli in a bench scale fermentor.

FIG. 19 shows a schematic diagram of an embodiment of the invention in which biomass, dissolved oxygen, and pH can be measured simultaneously.

FIG. 20 is a graph comparing pH curves in the microfermentor and in a 0.5 L bench scale fermentor (Sixfors).

FIG. 21 shows a schematic of a microfermentor integrated with optical density, dissolved oxygen, and pH sensors together with associated instrumentation and computer software.

FIG. 22 shows images of cells exposed either to an uncoated glass surface or to glass surfaces that were coated with various comb polymers. The central panel in the upper portion of the figure shows the molecular formula of the polymers.

FIG. 23 shows modeling of oxygen transfer in a microbioreactor as resistances-in-series.

FIG. 24 shows the modeled oxygen concentration profile across PDMS and membrane at t=0, 1, 2 hours (with cell growth modeled as exponential growth).

FIGS. 25A-25C show schematic diagrams of a microreactor of the invention that can be used for fed-batch fermentations. FIG. 25A shows an expanded view of the layer structure of the microreactor. FIG. 25B shows a longitudinal section of the microreactor with channels and integrated magnetic stirbar. FIG. 25C illustrates the principle of passive delivery of a liquid to the microreactor vessel.

FIGS. 26A and 26B show photographs of a realized embodiment of the microreactor of FIGS. 25A-25C. FIG. 26A shows a photograph of the empty vessel of the microreactor. The stirbar and fluorescent sensor for DO (black spot) are visible. FIG. 26B shows the microreactor vessel at the end of a fermentation run. Turbidity of the cell culture obscures the stirbar and the DO sensor.

FIG. 27 shows a schematic diagram of a top view of a microreactor of the invention with a plurality of channels extending from and in communication with the microreactor vessel and additional channels in the body layers that define the microreactor vessel and headspace.

FIGS. 28A-28C show schematic diagrams of the layer structure and sensor locations of the microreactor of FIG. 27, illustrating the path taken by 3 different channels, labeled A-A, B-B, and C-C.

FIG. 29 shows a photograph of a realized embodiment of the microreactor illustrated schematically in FIGS. 27 and 28A-28C.

FIGS. 30A and 30B show a schematic diagram of top and side views of a miniature magnetic stirbar useful to provide active mixing for certain microreactors of the invention. Dimensions are included for representative purposes and may be varied depending, for example, on the size of the microreactor.

FIG. 31 shows a schematic diagram of a set-up for operating a microreactor of the invention (in this case a microreactor with integrated stirbar and fed-batch capability). The diagram shows the instrumentation, optics, magnetic stirbar and actuating magnet, chamber in which microreactor is mounted, and fluidics for reagent feed and culture inoculation (syringe not attached during run). Components not drawn to scale.

FIG. 32A shows a schematic diagram of a body layer of a microbioreactor that can operate as a microchemostat and photographs of various components.

FIG. 32 B shows a schematic diagram of a body layer of a microchemostat with heated and cooled zones together with a temperature profile showing the temperatures of various regions of the device as determined using modeling.

FIG. 33 shows photographs of a realized embodiment of a microbioreactor that can operate as a microchemostat.

FIG. 34 shows a schematic diagram of a microbioreactor array in which the microbioreactors can operate as microchemostats.

FIGS. 35A and 35B show schematic diagrams of the layer structure of a microbioreactor that can operate as a microchemostat, including heated and cooled sections.

FIG. 36A shows a scheme for synthesis of comb polymers presenting long PEG chains grafted onto a poly(acrylic acid) (PAA) backbone. FIG. 36B shows a schematic diagram of a scheme for the modification of PMMA with a PAA-g-(PEG-r-PPG copolymer. x=grafting density.

FIGS. 37A-37D show that PEG modification increases resistance of PMMA and PDMA surfaces to cell adhesion. FIG. 37A shows adhesion of various cell types to unmodified (upper panels) and PEG-modified (lower panels) PMMA surfaces. Left panels show E. coli. Middle panels show S. cerevisiae. Right panels show fibroblasts. FIG. 37B shows a quantitative comparison of adhesion of various cell types to unmodified, PEG-modified, and PAA/PAAm multilayer-modified PMMA surfaces. Left panel shows E. coli adhesion. Middle panel shows S. cerevisiae adhesion. Right panel shows fibroblast adhesion. FIG. 37C shows images of PEG-modified (left) and unmodified (right) PDMS micochannels illustrating their relative wettability. FIG. 37D shows the resistance of PAA-g-(PEG-r-PPG)-modified surfaces to the non-specific adsorption of various proteins.

FIGS. 38A and 38B shows adhesion of E. coli to unmodified (A) and PEG-modified (B) PMMA surfaces after 1 day of culture in a microbioreactor.

FIG. 39 is a schematic diagram illustrating the concept of pressure-driven flow in a microchemostat.

FIG. 40 is a graph showing that variation in the stirring rate can control the oxygenation of medium in the culture vessel.

FIGS. 41A-41C show dissolved oxygen (DO), pH, and optical density (OD) of E. coli cultured in microbioreactors operating as microchemostats. FIG. 41A shows results obtained under oxygen-limited chemostat conditions (rich medium). FIG. 41B shows the same culture later in time and shows the effect of turning off the medium flow (resulting in non-chemostat conditions). FIG. 41C shows results obtained under oxygen-rich conditions, in which nutrients were limiting.

FIGS. 42A and 42B shows a microbioreactor of the invention. FIG. 42A shows a schematic perspective diagram of a microbioreactor with integrated sensors mounted on a glass substrate. FIG. 42B shows a photograph of the microbioreactor.

FIGS. 43A-43F are graphs showing values for bioprocess parameters monitored over time in microbioreactors and bench-scale bioreactors. FIGS. 43A and 43B show optical density in microbioreactors and bench-scale bioreactors respectively. FIGS. 43A and 43B show % dissolved oxygen in microbioreactors and bench-scale bioreactors respectively. FIGS. 43A and 43B show pH in microbioreactors and bench-scale bioreactors respectively. Each curve represents an individual bioreactor run.

FIGS. 44A-44D are graphs showing values for concentration of glucose (FIG. 44A), acetate (FIG. 44B), formate (FIG. 44C), and lactate (FIG. 44D) in a microbioreactor and a bench-scale bioreactor monitored over time.

FIGS. 45A-45C are graphs showing values for optical density (FIG. 45A), % dissolved oxygen (FIG. 45B), and pH (FIG. 45C) for cells cultured in microbioreactors with pure oxygen (open circles) or air (closed circles).

FIGS. 46A-46B show results comparing operation of batch and fed-batch fermentation runs in a microreactor capable of operating in fed-batch mode. FIG. 46A is a graph showing dissolved oxygen concentration over time in a fed-batch fermentation in which the culture (E. coli) was supplied with 4 g/L glucose (dashed line) and in a batch fermentation in which the culture was supplied only with water (solid line). FIG. 46B is a graph showing pH over time in two fed-batch fermentations in which the cultures (E. coli) were supplied with 0.1 M NaOH (dot-dash line) or 0.01 M NaOH (dashed line) and in a batch fermentation in which the culture was supplied only with water (solid line).

FIGS. 47A-47C show graphs of dissolved oxygen (DO), raw optical density (OD), and pH for four microreactors operating in parallel in an apparatus of the invention.

FIGS. 48A and 48B show graphs of optical density (OD), dissolved oxygen (DO) and pH of E. coli FB21591 grown in 50 ul microbioreactors in LB plus 0.8% glucose (A) and defined medium containing 0.8% glucose (B).

FIGS. 49A and 49B show graphs of dissolved oxygen (DO), optical density (OD), and pH for three microreactor fermentations operating in batch mode. FIG. 49A shows E. coli FB21591 cultured in LB+glucose+MES. FIG. 49B shows S. cerevisiae ATCC 4126 cultured in YPE+galactose.

FIG. 50A shows a schematic view of a longitudinal section of a microbioreactor suitable for continuous culture. FIG. 50B shows a photograph of the empty PMMA chamber of the reactor (middle layer of the 3 PMMA layers shown in FIG. 50A) with a magnetic stir bar in the center. FIG. 50C shows a Femlab simulation of temperature control and distribution in the microbioreactor.

FIG. 51 shows an experimental setup for a microbioreactor suitable for continuous culture.

FIG. 52 shows results of experiments in which E. coli were cultured under continuous culture conditions in the microbioreactor shown in FIGS. 50A and 50B. The figure shows attainment of steady state conditions at medium inflow rates of 0.5 μL/min, 1 μL/min, and 1.5 μL/min, as indicated.

FIG. 53 shows steady state conditions of pH, OD, and DO in E. coli cultures maintained under continuous culture conditions in the microbioreactor shown in FIGS. 50A and 50B at different dilution rates.

FIG. 54A shows a schematic view of a longitudinal section of another embodiment of a microbioreactor suitable for continuous culture. A-E, thermal bonded PMMA layers; F—PMMA cork; G—PDMS gasket and aeration membrane; H—silastic O-ring; I—optical fiber fixed by F; J—grit for holding PDMS membrane; K—magnetic mixer; L—PDMS optical plugs; M—optical fibers and micro-lens; N—fluidic interconnections; O—pH and DO fluorescent sensors. FIG. 54B is a photograph showing an overview of the individual parts. FIG. 54C is a photograph showing a bottom view of the microbioreactor with associated optical fibers and optical plug. FIG. 54D is a photograph of the assembled and bonded microbioreactor.

### DETAILED DESCRIPTION OF CERTAIN EMBODIMENTS

I. Overview

The present invention encompasses the recognition that microscale bioreactors (microfermentors) offer a means of addressing the continuing demand in bioprocess science and engineering for fast and accurate analytical information that can be used to rapidly evaluate the interactions between biological systems and bioprocess operations. In addition, such systems provide a platform for efficiently incorporating modern tools of biology (e.g., genetics, enzymology, molecular biology, and bioinformatics) to improve bioprocess screening and development. For example, microscale bioreactors allow the rapid screening of strains and metabolic pathways for applications ranging from synthesis of natural products to bioremediation. Bioprocess technology has been instrumental in the development and large-scale production of numerous pharmaceuticals and vaccines. In addition, bioprocesses are employed in the food industry, waste treatment, etc.

Metabolic pathway engineering is making a profound impact in areas as diverse as drug discovery (e.g., through the synthesis of novel natural products (2)), commodity chemicals (e.g., the synthesis of ascorbic and lactic acids (3) 1,3-propanediol (4)), and the biodegradation of toxic pollutants (5). Metabolic engineering encompasses the targeted improvement of product formation or cell properties through the modification of biochemical reactions. Hence, metabolic engineering focuses on determining the enzymes that offer the greatest amount of control over the rate of production of a certain metabolite (metabolic control analysis or MCA), then altering the activity of those enzymes (e.g., via molecular biology) and/or altering relevant reaction conditions to manipulate product yields. MCA can involve making mathematical models, carbon tracing, and developing assays for obscure metabolites and aids in the understanding of metabolic fluxes. The alteration of enzyme activities can involve polymerase chain reaction (PCR) techniques, genetic library construction, screening, cloning, and other molecular biology tools. Microfermentor technology will have a significant impact both on how bioprocess development and metabolic engineering research are carried out and also on how rapidly research can be translated into improvements into bioprocesses.

The invention provides microscale bioreactors that include a vessel for culturing cells having a interior volume of less than 200 μl and means for providing oxygen to the interior of the vessel so as to support the growth of cells. The terms “interior volume” and “working volume” are used interchangeably herein. In addition, the invention provides a microscale bioreactor system including a microscale bioreactor and a chamber that provides environmental control. The invention also provides a bioreactor assembly including an array of microscale bioreactors, which may be operated in parallel. The availability of a large number of bioreactors operating in parallel offers a number of unique advantages. For example, the microfermentor array makes it possible to (i) systematically evaluate the effects of varying one or more of a large number of parameters (e.g., temperature, nutrient composition, pH, etc.) on any phenotypic characteristic of interest, e.g., growth rate, metabolite production or compound biotransformation ability, etc., of a particular strain or (ii) systematically evaluate the characteristics (e.g., metabolite production) of a large number of different strains while holding environmental conditions constant.

Developing microscale bioreactors requires more than merely scaling down from currently available fermentor technology. For example, the large volumes employed in traditional fermentors makes it possible to monitor parameters such as oxygen concentration, biomass, etc., by removing samples from the fermentor at appropriate times. Sequential sampling may be impractical in the context of a microscale bioreactor or may need to be performed differently and on a smaller scale. Large indwelling sensor devices are not practical in the context of a microfermentor. Thus accurate monitoring of bioprocess parameters, a requirement for many applications, requires the development of alternative methods. Furthermore, oxygenation using traditional techniques such as sparging and/or stirring may be problematic in small volumes.

In addition to the challenges discussed above, use of fermentors with small volumes offers a number of potential advantages. For example, microfabrication technologies can be used to efficiently produce a large number of identical microfermentors. Microfabrication also allows integration of sensing devices into the structural components of the bioreactor, which enhances the possibilities for acquiring large amounts of data in an efficient manner. Thus in preferred embodiments of the invention at least one sensing device is integrated into a structural component of the microfermentor.

Miniaturization of fermentation processes to microliter scale represents a significant departure from conventional procedures. The inventors have recognized the need to address the following significant issues: (i) design and fabrication techniques, including materials selection and surface modification; (ii) bioprocess parameter control; (iii) selection, development, and integration of sensor technology; and (iv) appropriately sensitive analytical devices. In addition, the inventors have recognized the importance of utilizing appropriate biological systems for evaluating performance of the microfermentors and for comparing microfermentors with traditional bioprocessing methodologies. Significant differences between traditional fermentors and microfermentors include, for example (i) the ratio of wall surface area to volume; (ii) more significant evaporative losses in microfermentors; (iii) incompatibility of microfermentors with conventional oxygenation methods.

As described in more detail in the Examples, the inventors have constructed a microscale bioreactor with a working volume of 5 μl and have shown that it can support the growth of bacterial cells. The inventors have demonstrated successful delivery of oxygen to the microfermentor interior and lack of toxicity over a period of 10 hours.

Non-invasive online monitoring of dissolved oxygen, optical density, and pH during the culture period was achieved using integrated optical sensors. Results indicate that cell growth and various additional bioprocess parameters including dissolved oxygen profile and pH profile within the vessel over time, final number of cells, and cell morphology in the microfermentor are comparable to that in a conventional fermentor. Values of additional parameters including organic acid production and substrate utilization also closely resemble those obtained in larger fermentation vessels.

The inventors have constructed a number of additional microreactors having working volumes of less than 200 μl, including embodiments with magnetic mixers, and successfully employed them to monitor growth of microorganisms cultured in the microreactor vessels. In addition, the inventors have demonstrated a fed-batch system in which a solution of interest is added continuously to a microreactor during the culture period. Effects on cell growth were observed, demonstrating active control over bioprocess parameters. The inventors have also developed methods for measuring gene expression using the small growth volumes available from the microreactors of the invention.

The following sections provide relevant definitions, describe the manner in which the invention addresses the foregoing concerns and others, and describe methods for making and using the microreactors, microreactor arrays, apparatus for simultaneous operation of multiple microreactors, and other aspects of the invention.

II. Definitions

Actuating device (also referred to as an actuator): An “actuating device” or “actuator” refers to a device that puts another device or element of a system into action or motion.

Bioreactor Operation Strategies: In accordance with the terminology as commonly accepted in the art and described in (54), bioreactor operation strategies can be classified into one of three general modes, i.e., batch or fed-batch operations, the semi-continuous or cut-and-feed strategy (which may also be referred to as semi-batch), and perfusion culture. Batch culture is usually performed using suspension culture cells in a stirred tank bioreactor, although in the case of a microreactor as described herein, stirring may or may not be performed. Product is harvested from the medium at the end of the batch cycle. Fed-batch culture differs from batch culture in that nutrients (or solutions of interest such as reactants, buffers, etc.) are added either continuously or periodically during the batch cycle. The semi-continuous or cut-and-feed strategy also typically employs stirred tank, homogeneously mixed bioreactors. In this operating strategy a bioreactor is inoculated with cells, which are then allowed to grow for a period of time, often until the culture is approaching early stationary phase. A large fraction of the cell culture broth is then harvested, usually on the order of 70-90%, and the bioreactor replenished with fresh medium. The cycle is then repeated. Perfusion operations retain cells within the reactor while allowing a cell-free sidestream to be removed; they can be subdivided into two categories, the homogeneous systems such as the perfusion chemostat or heterogeneous systems like hollow fiber or fluidized bed bioreactors. It is to be understood that these definitions are not intended to limit the invention or its modes of operation in any way and that they are to be interpreted as appropriate in the context of microfermentors as described herein.

Channel: The term “channel” refers to a hole of constant or systematically varied cross-sectional area through a material. Generally a channel has a defined cross-sectional geometry, which may be rectangular, ovoid, circular, or one of these geometries with an imposed finer feature, such as indentations, etc. A “microfluidic channel”, also referred to herein as a “microchannel”, has at least one dimension of less than 1000 microns. Typically the characteristic dimensions of a cross-section of a microchannel (e.g., height and width of a channel with a rectangular cross-section, diameter of a microchannel with a circular cross-section, etc.) will both be less than 1000 microns. It will be understood that the cross-section is to be taken perpendicular to the length of the microchannel and that the length of the microchannel is often greater than 1000 microns. It will further be appreciated that any of the channels in the devices described herein may be, and typically is, a microfluidic channel.

Fermentation: The terms “ferment”, “fermentation”, etc., are to be understood broadly as indicating culture of cells in general. The terms do not imply any particular environmental conditions or metabolic processes. While typically these terms refer to culture of bacterial cells (e.g., eubacteria), they may also apply to archaebacteria or eukaryotic cells (e.g., yeast or mammalian cells). As a noun, a “fermentation” or “fermentation run” or “fermentor run” refers to a period of time during which cells are cultured in a fermentor.

Microreactor: As used herein, the term “microreactor” refers to a reactor, i.e., a device that contains a space in which a chemical or biochemical process (e.g., the growth of cells) is conducted, having an interior volume of less than 1 ml. Microreactors include microscale bioreactors, also referred to as microbioreactors.

Microscale bioreactor: As used herein the term “microscale bioreactor” or “microbioreactor” is used to describe a bioreactor (i.e., an apparatus for culturing cells) having an interior volume of less than 1 ml. The terms “microscale bioreactor” and “microfermentor” are used interchangeably herein.

Parallel: Reaction runs, including but not limited to, fermentor runs are performed “in parallel” when the run times of the runs overlap. The runs may, but need not be, started and/or terminated at substantially the same time. The runs may last for the same length of time or for different lengths of time.

Strain: In a broad sense, cells or viruses may be considered to be of different strains if they differ from each other in one or more phenotypic or genotypic characteristic. In general, a “strain” is a population of organisms descended from a single cell and maintaining the phenotypic and genotypic characteristics of that cell. Although frequently used to refer to microbes (i.e., microscopic organisms), the term may be used herein to refer to cells of any type.

III. Design and Fabrication of Microscale Bioreactors

A. Design

In certain embodiments of the invention the microscale bioreactor comprises a vessel for culturing cells and a means for providing oxygen to the vessel at a concentration sufficient to support cell growth. In certain embodiments of the invention the vessel has an interior volume of less than 1 ml. In certain embodiments of the invention the vessel has an interior volume of less than 200 μl. In certain preferred embodiments of the invention the working volume is between 50 μl and 100 μl inclusive. In certain preferred embodiments of the invention the working volume is between 5 μl and 50 μl, inclusive. In certain preferred embodiments of the invention the working volume is between 5 μl and 10 μl, inclusive. In certain preferred embodiments of the invention the working volume is approximately 7.5 μl or approximately 10 μl. In certain preferred embodiments of the invention the working volume is approximately 5 μl. (Generally the term “approximately” as used herein will indicate that a number may vary by ±1%, ±5%, ±10%, depending upon the context.) Small working volumes offer a number of advantages. For example, they permit efficient gas-liquid contacting to control the level of dissolved oxygen (DO). Small working volumes also imply smaller diffusion times, which aids in exchange of gases. In addition, microscale bioreactors having working volumes in the range of between 5 μl and 50 μl or between 50 μl and 100 μl may be more easily produced using microfabrication than those with larger working volumes. Microfabrication facilitates the production of microfermentor arrays with a very high density of individual microfermentors. In addition, microfabrication allows for configurations with very large specific gas-liquid interfaces. Particularly in the context of microscale bioreactors employing active aeration, microfabrication allows one to achieve a large mass transfer coefficient (k_{L}a). For example, the inventors have achieved a greater than two orders of magnitude increase in mass transfer coefficients for gas-liquid-solid reaction systems by precise design of the contacting scheme (8). Moreover, small system dimensions imply faster diffusion across the vessel volume and thus more uniform conditions within. Furthermore, smaller dimensions (e.g., dimensions resulting in an interior volume of less than approximately 100 μl) may be desirable to ensure adequate support for an aeration membrane that forms the top of the culture vessel.

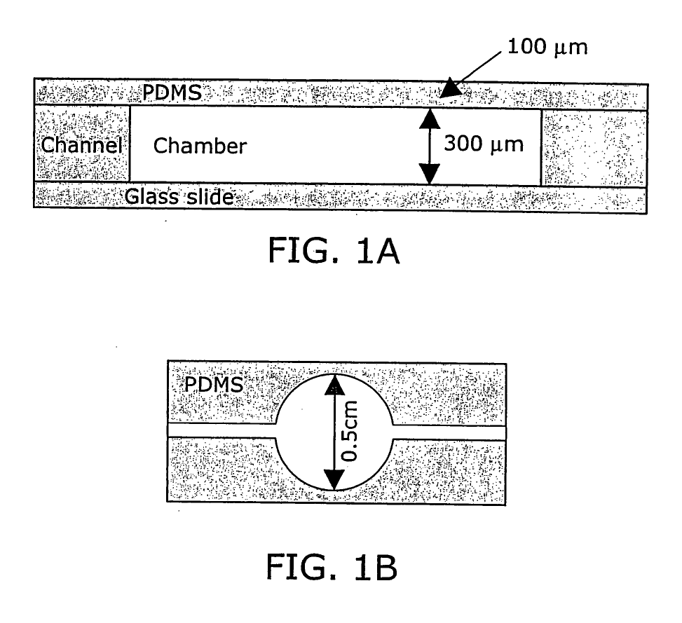

FIGS. 1A and 1B show top and side views of the design of one embodiment of a microfermentor of the invention. As seen in FIG. 1A, in this embodiment of the invention the vessel has a round cross-section in the horizontal dimension with an overall cylindrical configuration. The bottom of the microfermentor is formed from a rigid substrate (e.g., silicon, glass, plastics such as poly(carbonate), plexiglass, etc.), sufficiently strong to support and stabilize the remaining portions of the structure. In certain embodiments of the invention at least one wall (e.g., a side wall, top wall, or bottom wall) of the microfermentor comprises a transparent material to permit optical access. However, in certain embodiments of the invention use of a transparent material is not necessary as waveguides can be used to guide light in or out (see below).

As shown in FIG. 1, in preferred embodiments of the invention one or more channels extend from the vessel. For example, in those embodiments of the invention that operate in batch mode, the channels are used solely to introduce medium and inoculum (i.e., cells) to the vessel prior to the beginning of a fermentation. However, in certain embodiments of the invention such channels may be used for other purposes, e.g., to remove samples, to introduce additional components such as nutrients, buffers, etc., during the course of a fermentation. The channels may conveniently be used to interface with robotics, e.g., for introducing components into the vessel and/or for removing samples. Robotics may be used, for example, to interface microfermentors or microfermentor arrays with, for example, a microtiter plate from which materials may be transferred into the fermentor or into which samples may be placed. The channels may connect with pumps, reservoirs, etc. Microfluidics technology may be employed.

As described further below, the microfermentor includes means for delivering oxygen to the vessel. In preferred embodiments of the invention one or more walls of the microfermentor vessel consists at least in part of a gas-permeable membrane for oxygenation of the growing culture. The gas-permeable membrane may also aid in dispersal of gases produced during metabolism. In certain embodiments of the invention as described in Example 1, the membrane serves as both the aeration membrane and the structural material of the microfermentor. For example, as shown in FIG. 1, both the top and side walls of one embodiment of the microfermentor are made of the polymeric material poly(dimethylsiloxane) (PDMS). In certain embodiments of the invention the microfermentor includes multiple membranes. These membranes may be made from the same material or from different materials, e.g., materials having different properties such as gas diffusivity and solubility.

Since adequate oxygenation is a major consideration for cell growth, selection of appropriate microfermentor dimensions and membrane materials may be guided by an oxygen transport model that takes into account the properties of the oxygen delivery system. Use of such a model is described in more detail in Example 2. The calculations therein may readily be applied to any given material for which parameters such as oxygen diffusivity and solubility are known. In certain embodiments of the invention the permeability (i.e., product of diffusivity and solubility) of the membrane to oxygen is approximately equal to that of PDMS, i.e., 800 Barrer (1 Barrer=10⁻¹⁰ cm³(STP)·cm/cm²·s·cm Hg) (44). In certain other embodiments of the invention the permeability of the membrane to oxygen is greater than 800 Barrer. In certain other embodiments of the invention the permeability of the membrane to oxygen is either between approximately 600 and 800 Barrer, between approximately 400 and 600 Barrer, between approximately 200 and 400 Barrer, or between approximately 80 and 200 Barrer.

The invention provides a variety of microscale bioreactor systems in which two vessels are separated by a membrane. A first vessel serves as a cell culture vessel while the second vessel contains a liquid that serves as a source of one or more components such as oxygen, nutrients, buffers, etc. A variety of different configurations are possible.

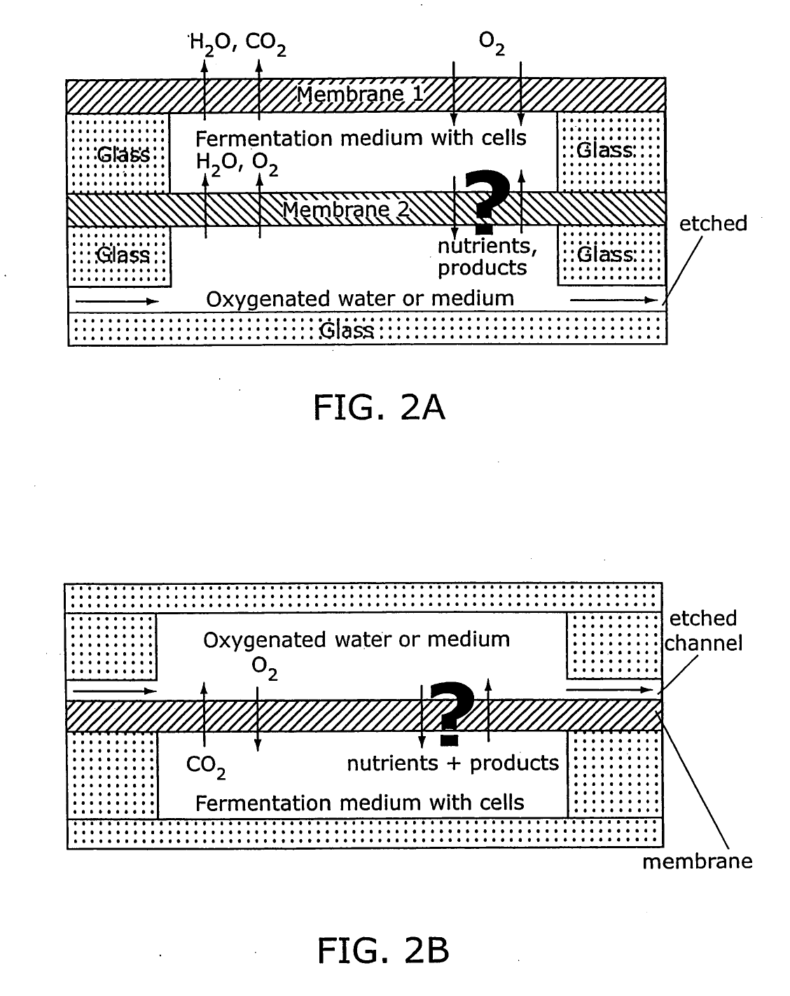

FIG. 2A shows a side view of one such embodiment of the invention in which the fermentation vessel is on top. The two vessels of the microscale bioreactor are separated by a membrane (Membrane 2) that allows free transport of water and oxygen into the top vessel. In certain embodiments of the invention this membrane prevents back-diffusion of nutrients, products, and/or salts while in other embodiments of the invention the membrane is permeable to these components. (The question mark in the figure indicates that nutrients, products, and salts may or may not diffuse through Membrane 2.) Membranes such as those typically used in desalination applications can be used for this purpose. A wide variety of membranes that may be used to control the transport of nutrients, products, salts, and cells is available from, e.g., Millipore Corp., Bedford, Mass. Factors such as pore size, surface characteristics such as hydrophobicity, and presence of channels for active or passive transport may be selected by one of ordinary skill in the art to achieve desired transport characteristics.

In the design depicted in FIG. 2A the top membrane (Membrane 1) allows diffusion of water and gases. Salts are not volatile so will not evaporate from the top membrane (Membrane 1), while most products are too large to diffuse readily through the top membrane. Channels in communication with the lower vessel allow oxygenated water to flow through the lower vessel, providing a continuous supply of oxygen and water to diffuse across Membrane 2. Circulation may be achieved using a pump. Since the liquid circulates and can be replenished, the volume of the lower vessel may be small relative to the volume of the upper vessel and may, in certain embodiments of the invention, consist merely of a chamber with similar height to that of the channels.

In certain embodiments of the invention rather than circulating liquid through a lower vessel as shown in FIG. 2A, a lower vessel with a volume that is large relative to the volume of the upper vessel (e.g., at least twice the volume of the upper vessel) is used, thus providing a reservoir of component(s). The contents of the reservoir may be replaced periodically. There may also be channels (not shown) in communication with the cell culture vessel, e.g., in order to allow introduction of cells and culture medium, removal of samples, etc.

This design offers the following features and advantages, among others: (1) Water losses from evaporation may be replaced by osmosis from bottom vessel; (2) Oxygenation may be provided from both the top and bottom (increases maximum allowable depth); (3) Contact with large reservoir of pH-neutral water or medium allows neutral pH to be maintained in the fermentor; (4) The process remains batch if only gases and water permeate membrane, while if the membrane allows nutrients, products, etc., to also permeate, process becomes semi-batch or continuous; (5) Since sensors may be integrated onto the glass or other material from which the microfermentor is fabricated, they are now separated from the fermentation medium. This allows separate calibration for sensors, and also eliminates need to sterilize sensors (e.g. some sensors are UV or temperature sensitive); (6) The design allows control of the oxygen gradient within the culture vessel by controlling oxygen content of water below, and atmosphere above, the culture vessel.

FIG. 2B shows another embodiment of a two-vessel microfermentor design. In this embodiment the culture vessel is not in contact with air. Instead, oxygen is provided via a membrane that separates the culture vessel from a second vessel that contains a reservoir of oxygenated liquid, e.g., water. The separating membrane allows free transport of water and oxygen into the culture vessel. In certain embodiments of the invention this membrane prevents back-diffusion of nutrients, products, and/or salts while in other embodiments of the invention the membrane is permeable to these components. (The question mark in the figure indicates that nutrients, products, and salts may or may not diffuse through the membrane.) Oxygenated liquid may be flowed through the upper vessel via channels as shown. In this design diffusion from the upper to the lower vessel takes place in the same direction as the gravitational forces.

This design offers the following features and advantages, among others: (1) Water losses from evaporation may be eliminated by contact with the water-filled vessel; (2) Contact with a large reservoir of pH-neutral water or medium allows neutral pH to be maintained in the fermentor; (3) The process remains batch if only gases and water permeate membrane, if the membrane allows nutrients, products, etc. to also permeate, process becomes semi-batch or continuous.

Although in FIGS. 2A and 2B the permeable membranes separating the two vessels have been depicted as structural components of the vessels, this need not be the case. The permeable membranes may instead form a portion of a separating layer made from a less permeable material.

In summary, the two-vessel designs address the potential problem of evaporative losses that may occur, e.g., in a non-humidified environment. In addition, these designs provide a second source of oxygen for the fermentation, and as a result a deeper culture vessel with a larger volume to surface ratio can be utilized. These designs also allow for control of pH, e.g., by allowing diffusion of protons and hydroxyl ions. In addition, pH control may be enhanced by providing appropriate buffers in the liquid that fills the second (non-culture) vessel.

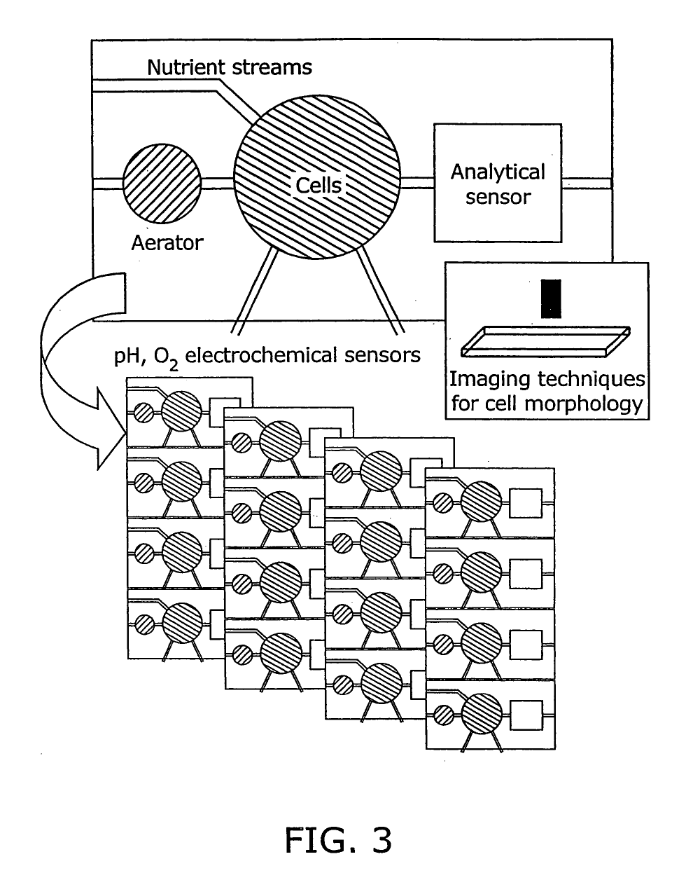

FIG. 3 shows a design of yet another embodiment of a microfermentor. The upper portion of FIG. 3 shows a single microfermentor unit. Each microfermentor includes a vessel in which cells are cultured and multiple channels extending from the vessel. The channels allow nutrient streams to enter the vessel and also provide means of contact between the interior of the vessel and various sensor devices. In this embodiment of the microfermentor, aeration is provided by means of a channel that allows communication between the microfermentor vessel interior and an external aeration chamber. This chamber may, for example, connect to a source of oxygen, may include a stirrer, etc. Multiple individual microfermentor units may be connected to a single aerator or each unit may have a dedicated aerator unit.

One of the goals of the invention is to provide an efficient platform in which multiple fermentations can be performed in parallel (e.g., simultaneously). Accordingly, the invention provides a system comprising a microfermentor array, by which is meant a plurality of physically connected microfermentors. The microfermentors are typically arranged in a regular geometry such as in mutually perpendicular rows, but this is not a requirement. Microfermentors are understood to be “physically connected” if they are arranged on or in a single substrate, attached to a common base, and/or connected to each other or to a central receptacle or chamber (e.g., via channels). The microfermentor arrays may include any number of individual microfermentor units. For example, in certain embodiments of the invention a microfermentor array includes at least 10 microfermentors. In certain embodiments of the invention a microfermentor array includes at least 100 microfermentors, at least 1000 microfermentors, or at least 10,000 microfermentors. The lower portion of FIG. 3 presents a sketch of an embodiment of a microfermentor array in which the individual microfermentor units shown in the upper portion of FIG. 3 are employed. (For illustrative purposes the columns are offset from one another.)

According to certain embodiments of the invention the system consists of multiple microfermentors, each with integrated bioanalytical devices, and operating in parallel. This system addresses the continuing demand in bioprocess science and engineering for fast and accurate analytical information that can be used to rapidly evaluate the interactions between biological systems and bioprocess operations. Moreover, the microfermentors provide the platforms for efficiently incorporating modern tools of biology (e.g., genetic profiling, enzyme catalysis, and bioinformatics) to improve bioprocess screening and development.

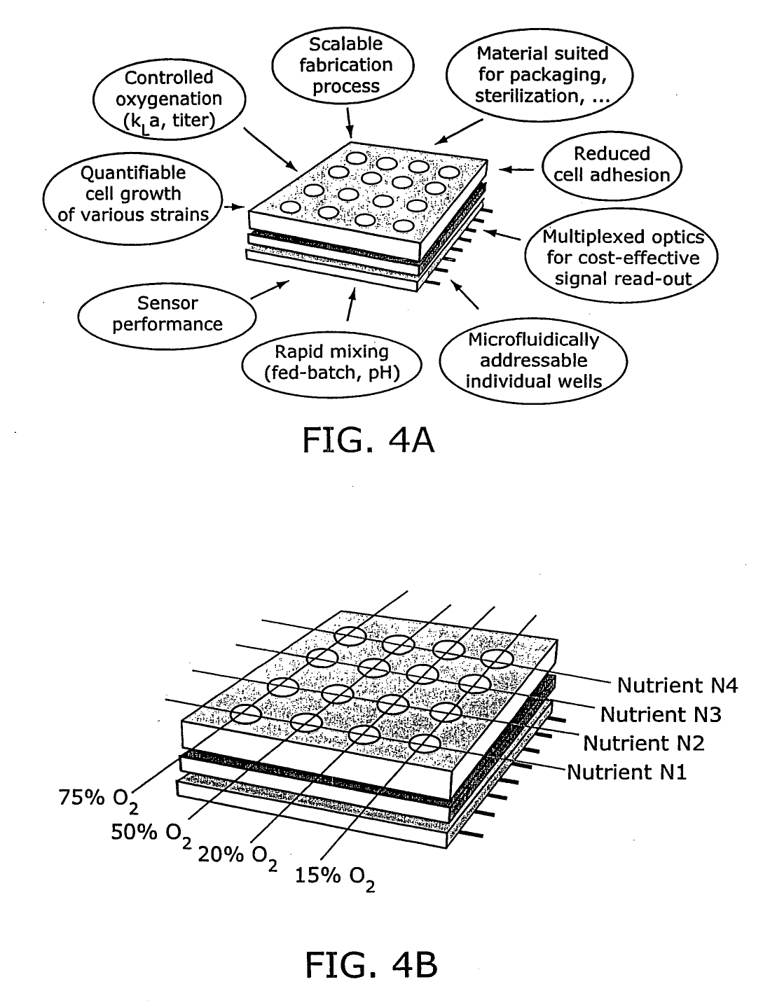

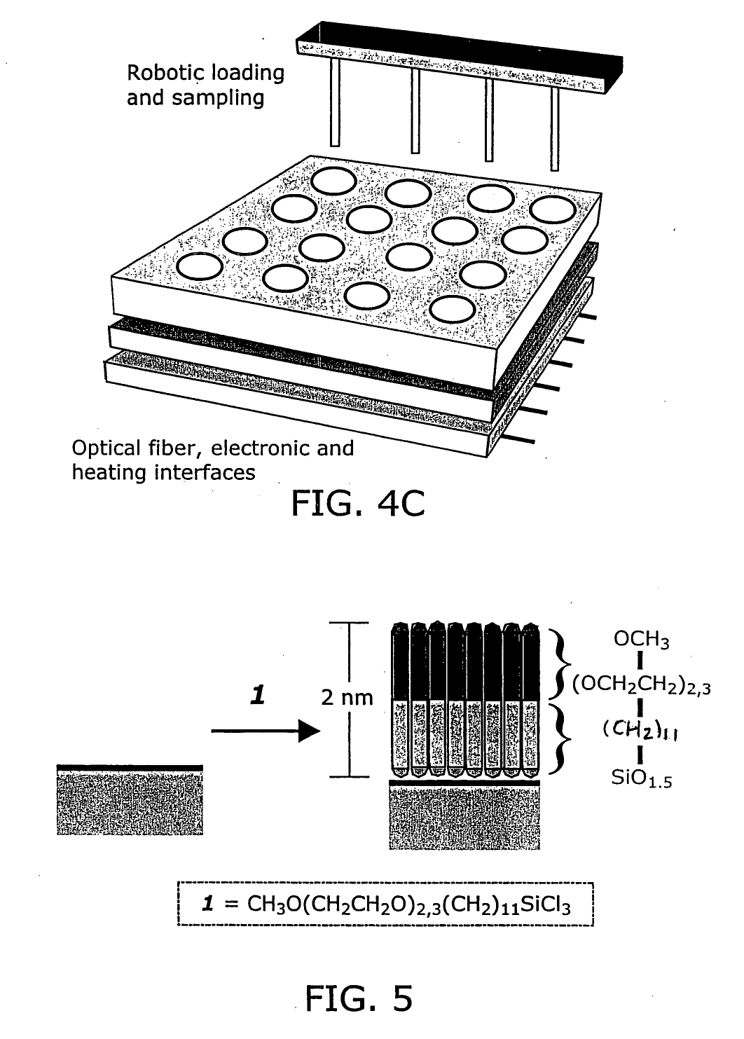

FIG. 4A is a schematic diagram of a system comprising an array of microfermentors consisting of mutually perpendicular rows and columns of individual units. Any of the microfermentors described herein may be either placed within the wells of the plate depicted in FIG. 4A or the wells themselves may serve as individual microfermentor vessels. According to certain embodiments of the invention the system allows for integrating parallel operation of multiple microfermentors with fluid delivery and optical and electronic sensing elements. The microfermentors can be run in different modes including batch, fed batch, and continuous. According to certain embodiments of the invention the microfermentor units can be autoclaved and exchanged.

The plate has chambers for multiple, parallel fermentation experiments. As shown in FIG. 4B, fluidic interface elements needed, for example, to inoculate the culture medium, to control pH, to add nutrient(s), or to remove portions of the cell culture may be integrated on the plate and in the system interface. This integration may be performed in such a way as to minimize mechanical manipulations and components needing sterilization. Elements present on or in the plate would typically include simple channels, valves, and connections to the system interface, etc. Other elements may also be included. Fluid control elements and delivery methods (e.g., pumps) may be housed in the system itself.

Similarly, according to certain embodiments of the invention reusable sensing elements are located elsewhere within the system whereas one-time use components are incorporated on or in the plate. For example, fluorescent dyes for dissolved oxygen and pH measurements may be incorporated into the plate, whereas optical fibers, lenses, and optical detection equipment may be situated in the system interface so that they could be used repeatedly for successive fermentation experiments. According to certain embodiments of the invention other means, e.g., optical means for measuring fluorescence and luminescence from biological species are incorporated into the system as described herein. Analogously, according to certain embodiments of the invention electronic sensing and automation means are incorporated into the system itself whereas simple actuator and sensing elements (e.g. electrochemical and capacitance) are incorporated into the plate.

According to certain embodiments of the invention the plate is packaged at the point of manufacture and may be pre-sterilized. When starting parallel fermentation, the plate is removed from the package and easily mounted in the system.

The plate and/or other system components can be manufactured by any of a number of standard microfabrication techniques, or combinations thereof, including but not limited to hot embossing, injection molding, electroplating, microelectrode discharge machining etc. According to various embodiments of the invention the plate is disposable or reusable depending, for example, on the particular application.

FIG. 4B is a schematic diagram of a system comprising a microfermentor array with microfluidic channels allowing control over parameters in individual microfermentors (see discussion of bioprocess control below). According to the approach depicted in FIG. 4B, by varying each of multiple parameters across different dimensions of the array, a combinatorial effect is achieved. For example, by employing four different values for dissolved oxygen and four different nutrient compositions across the two dimensions of the array, a total of 16 different culture conditions may be tested. According to various embodiments of the invention a single bioprocess parameter is varied across a single dimension of the array. According to certain other embodiments of the invention a plurality of bioprocess parameters are varied across one or more dimensions of the array.

Microfermentor arrays in which a plurality of substantially identical microfermentors operate in parallel offer a number of advantages. For example, it is possible to operate multiple microfermentors in parallel, terminate the fermentor run of one or more microfermentors at each time point of interest, and subject much or all of the contents of the microfermentor(s) to analysis. This offers an alternative to the approach of removing multiple samples from a single microfermentor, as would typically be done with a traditional bench-scale or industrial scale fermentor (although this approach may also be employed in the case of a microfermentor of the invention). The availability of multiple microfermentors operating in parallel thus offers higher flexibility for analysis.

The possibility of operating multiple microfermentors in parallel means that it will be possible to conveniently perform multiple substantially identical fermentation runs (e.g., multiple runs under identical or substantially identical conditions and/or in which the same organism is used) and to analyze the results of multiple such fermentation runs, which can greatly enhance confidence in the results. The degree to which conditions must be similar in order to be considered “substantially identical” may vary depending on the application and the particular condition under consideration. For example, two fermentation runs may be considered to occur under “substantially identical conditions” with respect to a particular parameter if the parameter varies between the two runs by less than approximately 20%, less than approximately 10%, less than approximately 5%, less than approximately 1%, or less than approximately 0.1%, depending, e.g., upon the particular parameter, the purpose of the fermentation run, etc. Rather than relying on results obtained from one or even a few large fermentations, the microfermentor arrays of the invention offer the possibility of obtaining data with increased statistical significance and of reliably identifying trends and variations, e.g., caused by different culture conditions.

In certain embodiments of the invention the microfermentor(s) and/or sensor(s) interface with standard laboratory robotics, with analytical equipment (e.g., HPLC, GC/MS, FTIR, etc.) and/or with data acquisition systems. In particular, in certain embodiments of the invention interfacing optical microscopy with the cell unit allows optical monitoring of cell morphology. In certain embodiments of the invention the microfermentors and microfermentor arrays are disposable.

The microfermentors, microfermentor arrays, and microfermentor systems of the invention may be mounted on or attached to a base and/or enclosed within appropriate housing. The housing may be provided with access ports, e.g., to allow entry and exit of wires, cables, tubes, etc. As used herein, according to various embodiments of the invention a “microfermentor system” includes one or more microfermentors or microfermentor arrays as described herein, optionally with associated microfluidic components, and one or more of the following: a plate or platform on or in which one more microfermentors or microfermentor arrays, optionally with associated microfluidics, may be mounted or housed; a chamber in which the microfermentors or microfermentor arrays, plates, or platforms may be enclosed; a pump; sensing and/or detection means; analytical equipment; robotics; software and computers, e.g., for data acquisition and/or bioprocess control; and any wires, cables, fibers, electronic components, etc., needed for operation of any of the foregoing system components. The system may include means for delivering energy to any component of the system, e.g., a

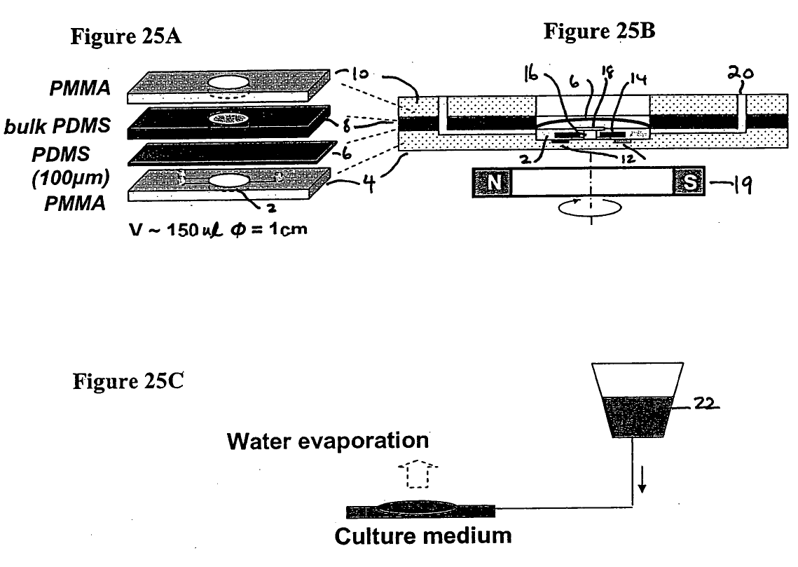

FIG. 25B shows a schematic diagram in longitudinal section of the design of another microreactor of the invention, which can supply one or more reagents to the vessel during operation. FIG. 25A shows an expanded view of a layer structure that can be used to implement the microreactor. Alternate structures and implementation approaches resulting in the same overall configuration may also be employed. As shown in FIGS. 25A and 25B, a microreactor vessel 2 is housed in first body layer 4. The material layers of the microbioreactor structure are relatively thin sheetlike expanses or regions of material that can be oriented so as to have substantially parallel upper and lower surfaces, the upper and lower surfaces generally having greater dimensions than the height. However, the layers can also assume a more blocklike or cuboidal shape, etc. In most cases the microreactors of the invention comprise multiple layers, overlying one another, but it is not necessary for a device to contain multiple layers.

A layer typically refers to a single thickness of a homogeneous substance, but the surfaces of the layer may contain depressions and/or outward projections. For example, as is evident from the figures and description herein, the culture vessels of the microbioreactors of the invention are typically fabricated as voids or depressions, also referred to as wells, in a material layer. The well may extend part of the way through the layer, in which case its bottom is formed by the remaining thickness of the layer. Alternately, the well may extend throughout the layer, in which case its bottom is formed by another layer beneath the layer containing the well. Layers referred to as “body layers” either contain a well or void that defines the interior of a culture vessel, or include a void having a similar shape to that of the culture vessel, so that in the assembled device the body layers are located such that the voids largely overly or lie beneath the culture vessel. Adjacent layers may contain complementary projections and depressions that fit together in the assembled structure.

Returning to FIGS. 25A and 25B, as shown schematically, in this embodiment of the invention the vessel exists as a void in body layer 4, but other methods of implementing the vessel are within the scope of the invention. A gas-permeable membrane 6 is located between the first body layer and a second body layer 8. The membrane extends across the vessel. An optional third body layer 10 overlies the second body layer. The surfaces of the body layers and gas-permeable membrane are preferably substantially planar, and upper and lower surfaces of each layer are substantially parallel to one another.

Voids in the second and optional third body layers provide the gas-permeable membrane with access to the external environment. By “external environment” is meant the environment immediately surrounding the structure from which the microreactor is fabricated. Thus if the microreactor is placed in a chamber, the external environment is typically the environment within the chamber. The external environment can be the environment within another device that interfaces with the microreactor, including devices that interface with the culture vessel, devices that interface with a channel, etc. For example, if flow between two channels A and B (or portions of a single channel) is regulated by a valve, the external environment of channel A can be the interior of channel B, and vice versa. In certain embodiments of the invention the first body layer and, optionally, the second body layer, are constructed out of a rigid material such as a rigid plastic, metal, etc., such that the layers do not bend under their own weight when supported at one end and do not deform during typical handling procedures. In certain embodiments of the invention the second body layer is constructed out of a less rigid material, e.g., the same material as the gas-permeable membrane. Thus the outer body layers provide resistance to damage that may occur, e.g., during handling, and protect the more delicate membrane and second body layers. The first body layer may be supported by a rigid substrate layer. Sensors (e.g., optical sensors) 12 may be mounted in depressions in the bottom of the microreactor vessel as shown in FIG. 25B or elsewhere in the vessel. The layers may be attached to one another using a number of different methods. They may be mechanically joined, e.g., using screws. Alternately, they may be bonded, e.g., using an adhesive or using thermal bonding or simply by positioning the layer between two or more layers whose positions are fixed with respect to one another. A combination of methods may be used.

Certain of the microbioreactors of the invention comprise a structural element or means for active mixing of the contents of the culture vessel. “Active mixing” is typically achieved using a device that converts electrical and/or magnetic energy into mechanical energy so as to cause motion of a mixing structure such as a stirbar, impeller, or other moving or rotating element, etc. A microbioreactor comprising such a mixing structure or any other structure or component that can perform active mixing of the contents of the culture vessel when supplied with an appropriate energy source is said to comprise a mechanism for actively mixing the contents of the culture vessel. The energy source may be located external to the microbioreactor device. The mixing structure is typically located in the microbioreactor culture vessel, though in some embodiments one or more mixing structures may be located in a microchannel and/or collection chamber. It will be understood that although the mechanism allows for continuous mixing of the contents of the culture vessel, e.g., during one or more culture periods, the use of the device is in no way limited to conditions in which continuous mixing is employed. It is also to be understood that the rate of mixing may vary or may be uniform during any particular period of time.

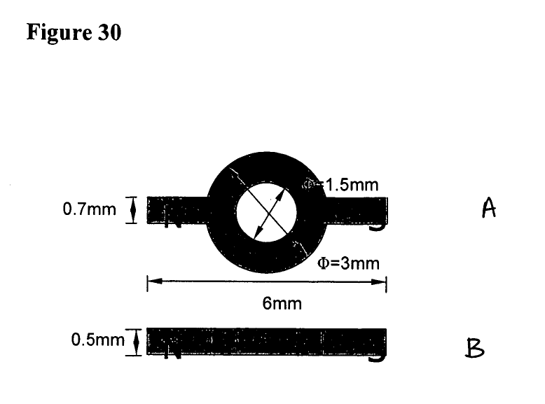

For example, as shown in FIG. 25B, the microreactor optionally includes a miniature magnetic stirbar 14, also referred to as a spinbar. The stirbar may be mounted on a vertical post 16 that projects upward from the base of the microreactor vessel. The post may be made out of the same material as any the lower body layer or may be made out of a different material. FIGS. 30A and 30B show schematic diagrams of top (30A) and side (30B) views of a stirbar suitable for use in the microreactor. North and south poles of the magnet project outward from a collar that is used to mount the stirbar on the post. In certain embodiments of the invention the magnetic stirbar is made of a material having a particularly high magnetic strength such as neodymium, neodymium-iron, neodymium-iron-boron, etc. As depicted in FIG. 25B, a cap 18 retains the stirbar on the post. The stirbar sits on a shoulder that is elevated a small distance (e.g, approximately 100-200 μm) from the bottom of the reactor. The shoulder serves to elevate the stirbar in the reactor for better spinning, prevent it from scratching the reactor bottom and from scratching optical sensors. This structure is optional. As discussed further below, rotation of the stirbar may be achieved by use of a rotating magnetic field, depicted schematically as magnet 19 below the microreactor.

One or more channels 20 (e.g., microfluidic channels) located within one or more of the layers extends from and communicates with the microreactor vessel. Such communication need not be continuous, e.g., there may be one or more valves located along the channel. The channels can be used to supply a variety of components to the microreactor vessel either before or during the microreactor run. For example, a first channel may be used to inoculate the culture with medium and cells (e.g., using a syringe). A second channel can be used to supply the vessel with a reagent during the run. Any of the channels may be blind in the sense of lacking an opening that communicates with the external environment following fabrication of the microreactor. Access to the channel may be gained by puncturing one or more body layers, e.g., with a needle. Certain materials will spontaneously reseal following withdrawal of the needle. Alternatively, it may be desirable to seal a channel using a material such as an adhesive. It may be desirable to include both types of channels, i.e., one or more channels that lacks a permanent communication with the external environment and one or more channels that includes a permanent communication with the external environment.

A variety of methods may be used to supply a reagent to the interior of the microreactor vessel, including both active and passive pumping strategies. FIG. 25C illustrates the principle of passive delivery of a liquid to the microreactor vessel. A reservoir 22 containing a liquid is provided and is connected to the vessel via a channel and appropriate tubing if necessary. Preferably the reservoir is located at an elevated position with respect to the vessel. Evaporation of water from the culture medium draws liquid from the reservoir into the channel and drives it into the vessel. The microreactor can be operated as a batch process when water is fed into the vessel from the reservoir or in a fed-batch mode when a reagent such as a nutrient (e.g., glucose), base, etc., is placed in the reservoir. This approach can be utilized for microreactors operating in parallel, in which case it may be desirable to provide a single reservoir connected by channels to the individual microreactor vessels.

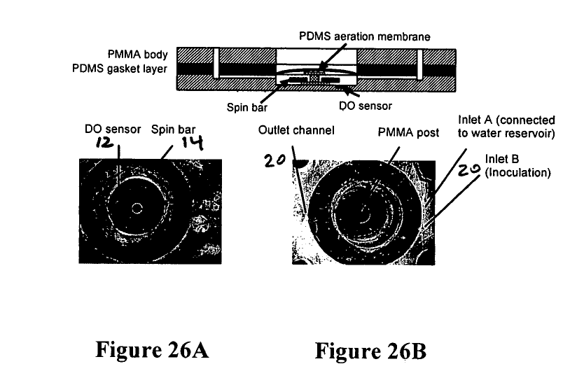

FIGS. 26A and 26B show photographs of a realized embodiment of the microreactor described above. FIG. 26A shows the microreactor with an empty vessel. A DO sensor 12 and stirbar 14 are visible as are three microfluidic channels 20. FIG. 26B shows the same microreactor, following a fermentation run. The channel inlets for connection to a reservoir and for inoculation are indicated. Turbidity of the culture obscures the sensor and stirbar.

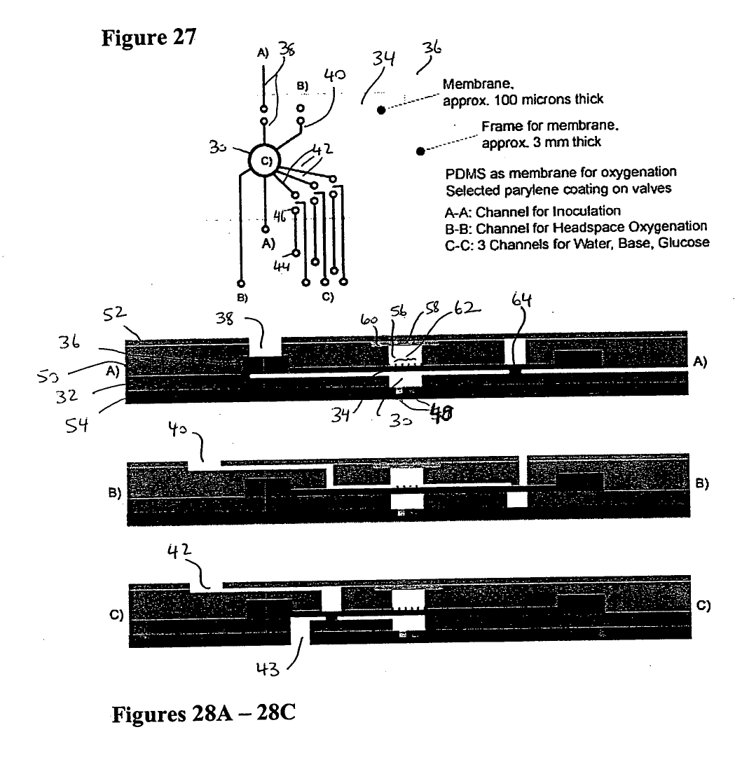

FIG. 27 shows a schematic diagram of a sectional view of another microreactor of the invention. The section is taken primarily in the plane of a gas-permeable membrane layer as described below, but certain elements such as the reactor vessel and channels, which are present in other layers are also depicted. The figure is color coded, with the colors representing elements that are present within different layers as shown in FIG. 28. For example, green represents elements in FIG. 27 that are present within the green layer in FIG. 28. FIGS. 27 and 28A-28C are most easily understood if considered together. In both figures, a plurality of channels communicate or potentially communicate with a microreactor vessel 30, which is housed in a body layer 32. A gas-permeable membrane 34 extends across the vessel. The membrane is optionally secured by another body layer 36, which serves as a frame or gasket for the membrane. The channels include channel 38, which extends from points marked A to A in FIG. 27, channel 40, which extends from points marked B to B in FIG. 27, and channels 42, which extend from points marked C to C in FIG. 27.

The open circles in FIG. 27 represent blind termini, which may be voids or holes in one or more layers of the structure. Access can be gained to channels connected to blind termini, or termini can be joined to one another, either by puncture with a needle or by opening a valve. The termini depicted in FIG. 27 may be located in different layers of the structure. For example, terminal 44 and terminal 46 are located in different layers, with terminal 44 located in a layer directly above the layer that contains terminal 46. A needle inserted at terminal 44 can be used to pierce through to terminal 44, thereby providing access to the vessel interior. It will be appreciated that when this method is used, the region to be pierced should be made out of a material that can be readily pierced.

FIGS. 28A-28C show 3 cross-sectional views of the layer structure of the microreactor of FIG. 27. The microreactor includes first body layer 32, gas-permeable membrane 34, second body layer 50, third body layer 36, a fourth layer 52 that overlies the second body layer, and optional substrate layer 54. Sensors 48 for measuring bioprocess parameters (e.g., oxygen, pH) in the vessel are embedded in the substrate layer but may be positioned elsewhere. The microreactor vessel is located within the first body layer. The third body layer serves as a gasket for the gas-permeable membrane. By “gasket” is meant a device used to retain fluids under pressure or seal out foreign matter, e.g., a seal made from a deformable material and compressed between plane surfaces. A void in the second body layer defines a headspace 56 for the microreactor vessel, by which is meant an empty space that does not contain liquid (but may, of course, contain gas). A sensor 58 (e.g., a carbon dioxide or oxygen sensor) is located in communication with the headspace for sensing the contents thereof. The sensor may be embedded in a protective structure 60 (e.g., a Teflon ring surrounding the sensor). The fourth layer serves a protective function. The sensor may optionally be embedded in this layer. Sensors may also be placed within any of the channels. In order to prevent bulging of the gas-permeable membrane upwards into the headspace (which may occur if there is even a minor pressure difference as depicted schematically in FIG. 25B), an optional element 62 may be included. Element 62 may be, for example, a grid composed of the same material as body layer 50 or of a different material. The configuration of the element may vary, provided that it does not excessively prevent gas transfer across the gas-permeable membrane. It is generally desirable to reduce or minimize bulging of the membrane since such bulging can affect the accuracy of optical measurements. A variety of different methods can be used to reduce bulging.

FIG. 28A shows the path taken by channel 38 through the microreactor structure. Channel 38 may be used, for example, to inoculate the vessel with media containing cells. Starting near the left side of the figure, channel 38 enters the structure through voids in layers 52 and 50. The channel then encounters body layer 36, which must be pierced to allow access to the next portion of the channel. Continuing below, in body layer 32, the channel provides access to the microreactor vessel. The channel continues to the right of the vessel and comes to valve 64. The valve may be, for example, a portion of body layer 32 that projects upward into the channel. Pressure in the channel causes the overlying membrane to move upwards into void 66, thereby allowing fluid to flow beyond the valve into the rightmost portion of the channel. Valve 64 may be used to allow flushing of the microreactor vessel. Similar pressure-operable valves may be present elsewhere in the structure. Other types of valves may be used instead. It will be appreciated that a valve such as valve 64 can be actuated by applying pressure from either the left or right side of the valve.

FIG. 28B shows the path taken by channel 40 through the microreactor structure. Channel 40 may be used, for example, to supply oxygen to the headspace and/or to flush the headspace. Starting near the left side of the figure, channel 40 enters the structure through a void in layer 52 and continues in layer 50. The channel extends down through layer 50 until encountering membrane 34. It continues leftward and enters the headspace. The channel exits the headspace on the opposite side and ends blindly. However, the portion of the membrane below the channel can act as a valve, being displaced downwards into the void below (in layer 32) when pressure is applied at the other end of the channel. Fluid may thus be forced through the channel, exiting through the void in layers 50 and 52 near the left end of the channel.

FIG. 28C similarly shows the paths taken by channels 42 and 43 through the microreactor structure. Channels 42 and 43 may be used, for example, to supply a reagent to the microreactor, e.g., during a fermentation run. As described above, displacement of portions of the gas-permeable membrane acts as a valve allowing fluid to enter the portion of the channel in direct communication with the interior of the microreactor vessel.

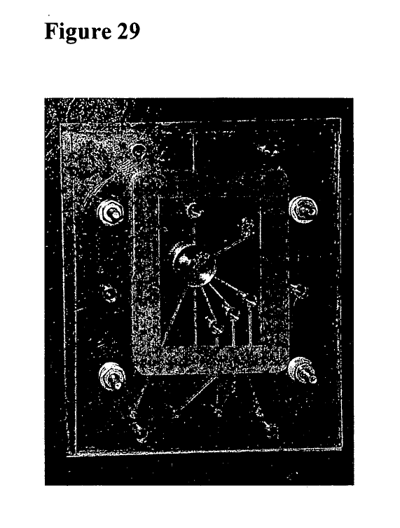

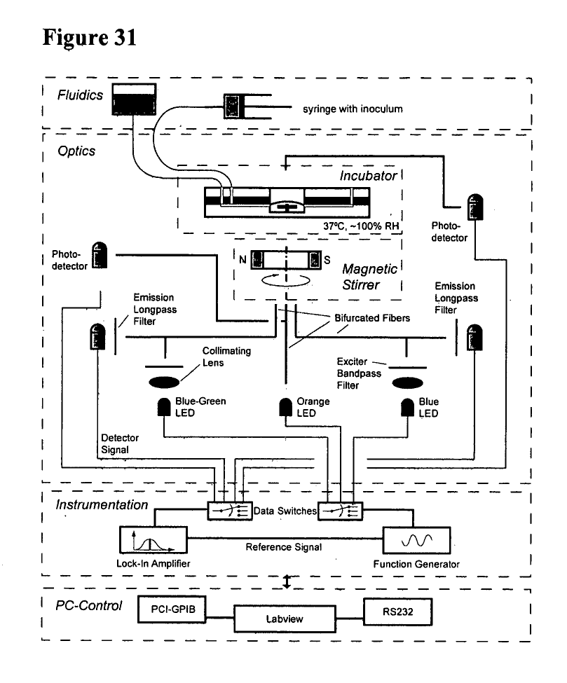

FIG. 29 shows a photograph of a realized embodiment of the microreactor of FIGS. 27 and 28. The microreactor vessel, gasket layer and various channel elements are visible. The microreactor may optionally be provided with a stirbar as described above. Additional components such as reservoirs for feeding reagents, an oxygen supply, etc, may also be provided. The microreactor may be operated in a set-up such as that depicted in FIG. 31A, which shows a microreactor structure with integrated stirbar and actuating magnet, connected fluidics that interface with one or more channels, and a syringe for inoculation. Optical elements for signal transmission, excitation, and detection are also depicted and are described in more detail elsewhere herein. Such elements may measure transmission, absorption, reflection, fluorescence, luminescence, etc.

Certain of the microbioreactors described above may be operated as microchemostats. These microbioreactors allow for continuous medium inflow and outflow and allow for precise control over growth conditions within the culture vessel. As is known in the art, a chemostat is a continuous culture system in which the supply of nutrients is determined externally and cell growth and/or biomass increase is limited by the availability of a selected nutrient. Either prokaryotic (bacteria) or eukaryotic (e.g., fungal, insect, mammalian, etc.) cells can be cultured in a chemostat. The growth-limiting nutrient can vary and is often a carbon source such as glucose, but can also be other nutrients, such as nitrogen source, specific amino acids, nucleotide precursors, trace minerals, etc. For purposes of the present invention, growth can also be limited by factors other than nutrient availability. For example, the growth-limiting factor may be the presence of a gas such as oxygen. pH or temperature can also be growth-limiting. In general, any factor that affects cell growth and can be externally controlled and maintained at a fixed level can be the growth-limiting factor in a chemostat. Generally nutrient availability is controlled by supplying a constant flow of medium of a given composition to a culture vessel and removing culture medium from the vessel at an equal rate (i.e., volume/time). Thus the microbioreactor can comprise means for supplying a constant flow of medium to the culture vessel and means for removing culture medium from the vessel at an equal rate to the rate at which medium is supplied. Such means should be capable of operating while the microbioreactor is being used to culture cells.

Chemostat operation is often described in terms of the dilution rate D, which equals the flow rate F (volume/time) divided by the culture volume, V. The dilution rate, D, equals the specific growth rate, u, a measure of how fast a cell reproduces that reflects the intrinsic ability of the cells to reproduce under the given conditions. See Smith, H. L., et al, The Theory of the Chemostat: Dynamics of Microbial Competition (Cambridge Studies in Mathematical Biology), Cambridge University Press, Cambridge, England (1995) for additional details regarding chemostats and some of their uses.

“Constant growth conditions” or “chemostat conditions” refers to a situation in which environmental conditions that are physiologically relevant for cell growth are maintained at a fixed level (to within experimental error) so that on a statistical basis cells in the culture are exposed to an identical and constant environment over time. The biomass concentration and/or cell density thus remains constant within the culture vessel for a prolonged period of time, and the culture is in a steady state. It is noted that biomass concentration refers to weight of cells per unit volume (either dry or wet weight can be used), while cell density refers to the number of cells per unit volume. In many instances these parameters are directly related and can be used interchangeably, though exceptions exist such as situations in which cell division is inhibited, in which case cells can increase in volume but cannot divide. Another example is a population of cells that is synchronized with respect to cell cycle stage, in which case there can be an increase in total cell volume without an increase in cell number during G1, S, G2, and/or M phase and a sudden increase in cell number without a correspondingly large increase in total cell volume when cytokinesis takes place.

The growth conditions can include concentration of dissolved gases (e.g., oxygen, carbon dioxide), the pH, the temperature, the biomass concentration, the cell density, the concentration of one or more nutrients, the concentration of one or more metabolic products, or any combination of the foregoing. By “prolonged period of time” is meant at least 5 times the turnover time (i.e., the time that would be required to completely fill an empty culture vessel), which is numerically equal to the reciprocal of the dilution rate. Preferably growth conditions and biomass concentration remain constant for at least 10 times the turnover time, more preferably at least 20 times the turnover time, yet more preferably at least 30 times, at least 50 times, at least 100 times the turnover time, or longer. It is important not only that the average concentrations of nutrients, oxygen concentration, etc., within the culture vessel remains constant but also that the contents of the vessel are well mixed, in order to avoid local differences in growth conditions.

The existence of constant growth conditions can be verified by assessing parameters such as dissolved oxygen concentration (e.g., as a percentage relative to the dissolved oxygen concentration that exists when medium without cells is in room air), pH, and biomass concentration (e.g., cell density) over time. Typically, the rate of change (dX/dt), of these 3 parameters (where X is dissolved oxygen concentration, pH, optical density) is less than 0.25, more preferably less than 0.1, and more preferably less than 0.05, and still more preferably less than 0.01 over a prolonged period of time to verify the existence of chemostat conditions. In the case of an anaerobic culture (e.g., a culture of strictly anaerobic cells), the dissolved oxygen concentration should be approximately 0. Appropriate corrections can be made for artifacts and/or measuring errors due, for example, to transient changes in the volume of medium in the culture vessel due to minor fluctuations in pressure driving medium inflow and outflow. Measuring the concentrations of various nutrients and/or metabolites (either online or offline) can also be used to verify the existence of constant physiological conditions. Comparing gene expression profiles over time provides a complementary approach that may be used to verify the existence of constant physiological conditions.

While, a chemostat may be inoculated with only a single cell, in practice it is more typical to inoculate with a plurality of cells and to maintain chemostat conditions in a culture vessel with a plurality of cells. For example, a chemostat such as the microchemostats of the invention may be inoculated at a density of at least 10/ml, at least 10² cells/ml, at least 10³ cells/ml, at least 10⁴ cells/ml, at least 10⁵ cells/ml, at least 10⁶ cells/ml, at least 10⁷ cells/ml, or more. Preferably chemostat conditions are maintained for a prolonged period of time at cell densities of at least 10 cells/ml, at least 10² cells/ml, at least 10³ cells/ml, at least 10⁴ cells/ml, at least 10⁵ cells/ml, at least 10⁶ cells/ml, at least 10⁷ cells/ml, or more. In certain embodiments of the invention the chemostat is inoculated and/or maintained at a cell density of between 10 and 10⁸ cells/ml, or within any range intermediate between these two values.

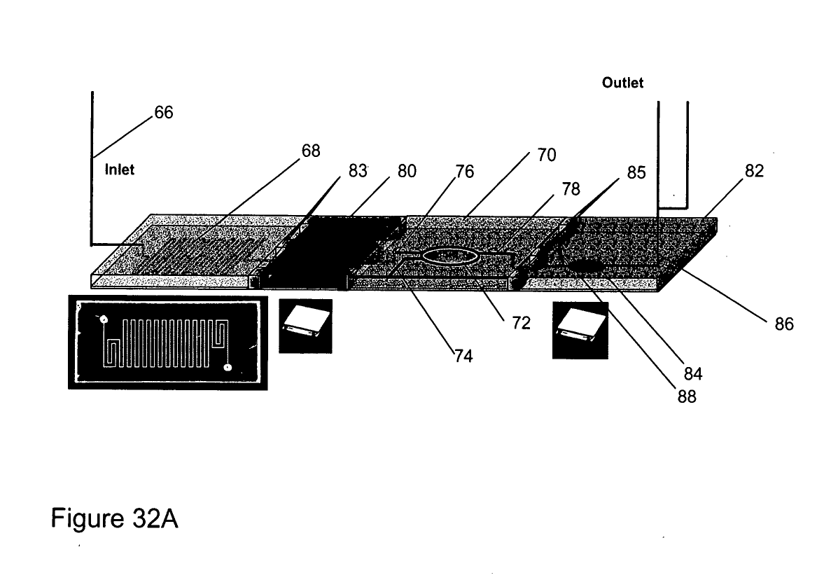

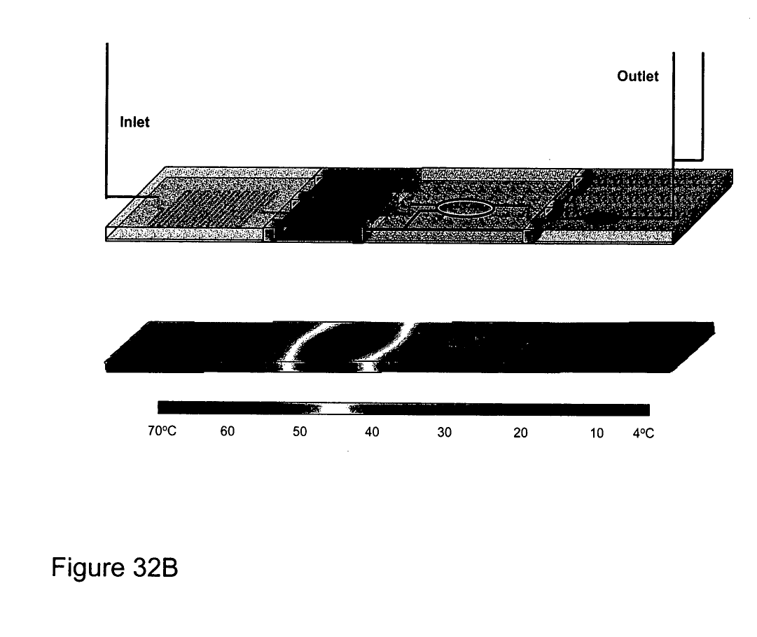

FIG. 32A shows a schematic diagram of a microbioreactor that can be operated as a microchemostat (i.e., a chemostat in which the interior volume of the culture vessel is less than 1 ml). As in the case of the other figures herein, it is to be understood that the figure itself and description thereof represent various embodiments of the invention and are not intended to be limiting. Inlet 67 represents a connection (e.g., a length of tubing) to a medium reservoir (not shown), which joins channel 68 in the device. Channel 68 can, but need not, have a winding configuration along all or part of its length, with multiple turns and bends as shown. This configuration results in a channel with considerably greater actual length than the distance of a single straight line extending between the beginning and end of the winding portion. Having a longer effective length results in a lower medium flow rate than would otherwise be the case when a constant pressure source is used to drive medium flow and thus avoiding the need to set the pressure at extremely low values.

The device includes body layer 70, which contains the culture vessel 72. The gas-permeable membrane which covers the opening of the culture vessel and would be present in a fully assembled microbioreactor is not shown. The culture vessel contains means for active mixing, e.g., a magnetic stirbar (not shown). Inoculation channel 74 is in communication with the culture vessel via an inoculation port which in this case is simply the junction at which the inoculation channel opens into the interior of the culture vessel but could also be a discrete structure. Medium inflow channel 76 and medium outflow channel 78 are in communication with the culture vessel via medium inflow and medium outflow ports, which could also be discrete structures but in this case are simply the junction at which the channels meet the culture vessel, through which fluid can flow.

The device depicted in FIG. 32A consists of a plurality of sections. The term “section” is intended to indicate a portion of a structure that is distinguishable from one or more other portions of the structure, e.g., it is at least in part physically or materially discontinuous with, separated from, or spaced apart from, one or more other portions of the structure. Different sections may be fabricated as a single structural unit containing, for example, gaps, spaces, boundaries, etc., or may be fabricated as separate units that are then assembled.

Medium inflow and outflow channels 76 and 78 extend from the section that contains the culture vessel to adjoining sections 80 and 82 on either side that are spaced apart, but physically connected with, the section containing the culture vessel. The section containing the vessel and the adjoining sections are physically connected by connecting elements 83 so that continuous channels can be formed that allow fluid to flow from one section to another. In the embodiment shown in FIG. 32A, a single channel flows between sections 80 and 82 via the connecting element in the center, joining channels 68 and 76, but multiple channels could flow through a single connecting element or through multiple connecting elements. Channels 68 and 76 could also be considered a single continuous channel with multiple segments but have been numbered separately for purposes of convenience. The adjoining sections are depicted as being in the same material layer as the culture vessel but need not be, i.e., they could be at least in part in a different plane. In the embodiment shown in FIG. 32A, the sections are joined by connecting “bridges” such as connecting elements 83. The region between the connecting elements is empty but in certain embodiments of the invention it is filled, e.g., with an insulating material. Rather than having discrete connecting elements, the space between adjacent sections is filled with a different material in certain embodiments of the invention.

The adjacent sections provide spatially distinct regions in which environmental conditions that differ from those present in the culture vessel can be established and confined so that they do not substantially affecting environmental conditions within the culture vessel. A variety of other configurations could be used to provide spatially distinct regions, provided that they sufficiently isolate the spatially distinct regions from the culture vessel. In the context of the microbioreactors of the present invention, environmental conditions that affect cell growth, movement, metabolism, etc., may be established within the spatially distinct regions. When operating as a microchemostat, a continuous flow of culture medium into the culture vessel must be maintained, necessitating the existence of a continuous connection to a source of medium. It is of particular importance to prevent contamination of the medium reservoir and conduits leading from the medium reservoir into the culture vessel. Under conditions in which nutrients, oxygen, etc., are limiting in a culture vessel bacterial chemotaxis (directional movement in response to a chemical stimulus such as a gradient of a nutrient) will tend to occur, which would result in the movement of bacteria out of the culture vessel and into the medium inflow channel, where a higher concentration of the limiting factor exists. Ultimately bacteria may reach the medium reservoir.

The inventors have recognized that the problem of contamination may be addressed by establishing a spatially distinct region in which conditions inhibitory to cell growth and/or movement exist, such that culture medium must flow through the spatially distinct region in order to enter the culture vessel and cells must pass through the spatially distinct region in order to reach the medium reservoir from the culture vessel. Chemotaxis of most bacterial species can be inhibited by heat. In the embodiment depicted in FIG. 32A, spatially distinct section 80 is heated, preferably to at least about 50° C., more preferably 50-60° C., yet more preferably 60-70° C. Higher temperatures, e.g., 70-80° C. or more could also be used. Such temperatures can substantially prevent bacterial chemotaxis for most bacterial species, and the temperature can be selected such that any cells entering the spatially distinct region are killed. In order to reduce heating of the medium entering the culture vessel, the proximal portion of the medium inflow channel (closer to the conduit that connects to the medium reservoir) is located in a separate section spaced apart, though continuous with, the heated section. Channel dimensions and flow rate can be selected to avoid excessive heating of the medium that flows through the heated section to reduce the potential destructive effect of heating on components in the medium (e.g., antibiotics) and to reduce any potential effects of the heated medium on temperature within the culture vessel.

In some embodiments of the invention the microbioreactor does not include separate sections, but the dimensions and positions of one or more inflow channels (e.g., a medium inflow channel) and the culture vessel are such that it is possible to maintain environmental conditions in at least a portion of an inflow channel which are different from those that are maintained within the culture vessel, without significantly affecting the conditions in the culture vessel. For example, as described in Example 12, part of the microbioreactor structure through which the medium inflow channel passes can be heated to a temperature sufficient to substantially inhibit bacterial chemotaxis and/or kill bacteria that enter the heated zone without significantly affecting the temperature in the culture vessel.

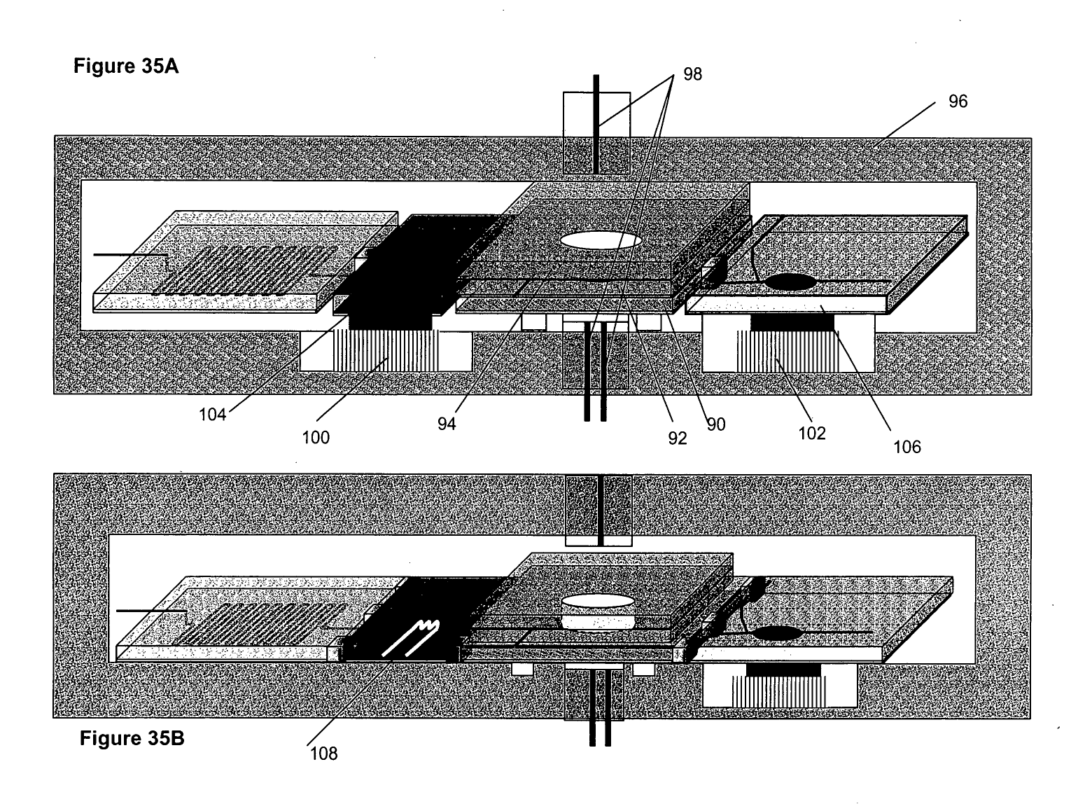

Any suitable external heating device can be used, an example of which is shown in the photograph in FIG. 32A underneath section 80, and discussed further in Example 10. Alternately, in some embodiments of the invention a heating coil is embedded in the spatially distinct region (see FIG. 35B).

Maintaining a relatively high average linear flow rate (fluid volume/(cross-sectional area of channel)(time)) in the medium inflow channel also reduces movement of cells towards the medium reservoir. The maximum swimming speed of a number of different bacteria is approximately 100˜125 μm/s, and the average swimming speed is typically approximately 30 μm˜50 μm/s. In certain embodiments of the invention the average linear flow rate in the medium inflow channel is at least 30 μm/sec, between 30 and 50 μm/sec, between 50 and 100 μm/sec, between 100 and 500 μm/sec, between 500 and 1000 μm/sec, between 1000 and 2000 μm/sec, or within any intermediate range. Higher values could also be used. Lower values are also usable, particularly if the medium inflow channel also passes through a region that inhibits cell growth and/or movement. One of ordinary skill in the art will be able to select appropriate channel dimensions and volume flow rates to achieve a wide range of average linear medium flow rates. For example, Example 10 describes channels having a 20 μm×250 μm cross-section, in which the average linear flow rate is 200 μm/sec at a volume flow rate of 0.8 μL/min and 500 μm/sec at a volume flow rate of 2 μL/min. Example 12 also describes suitable channel cross-sectional dimensions and flow rates. Modeling (e.g., using FEMLAB® or other fluid dynamics programs) could be used to test various channel dimensions and flow rates. It is noted that the liquid flow rate in a channel such as those of the microbioreactors of the invention generally assumes a parabolic distribution, with faster rates near the center of the channel and slower rates closer to the walls. Unless otherwise indicated, linear flow rates described herein refer to average linear flow rates. One of ordinary skill will be able to select appropriate channel dimensions and volume flow rates to achieve a wide range of maximum or minimum linear medium flow rates. In certain embodiments of the invention the average linear medium flow rate is selected to be at least as great as the average or maximum swimming speed of cells (e.g., bacteria) to be cultured in the microchemostat. In certain embodiments of the invention the minimum linear medium flow rate is selected to be at least as great as the average or maximum swimming speed of cells (e.g., bacteria) to be cultured in the microchemostat.

Medium leaving the culture vessel flows through spatially distinct region 82, which is depicted as a section adjoining and physically connected to the section containing the culture vessel via connecting elements 85. This section contains a collection chamber 84, in communication with medium outflow channel 78, which continues beyond the collection chamber and ends at outlet 86. It will be appreciated that the media flow channels leading into and out of the collection chamber could be considered to be individual channels or segments of a single channel, connected via the collection chamber. The collection chamber can be of any convenient volume, typically at least 10% of the volume of the culture vessel and can be fabricated as a well in either the upper or lower surface of the material, provided that another layer exists either above or below, respectively, to enclose the chamber. Channel 88 can be used to withdraw a sample from the collection chamber and/or to introduce a fluid into the collection chamber. Outlet 86 is in communication with an effluent reservoir (e.g., via a length of tubing). Sample may also be removed from the collection chamber via outlet 86, for which purpose it may be useful to have a bifurcated conduit communicating with the effluent reservoir, as shown in FIG. 32A. Any of the channels or conduits may be provided with valves.

The collection chamber may be located in a spatially distinct region in which environmental conditions that inhibit cell growth and/or reduce cell metabolism are established. This can be achieved, e.g., by cooling the spatially distinct region to an appropriate temperature, preferably less than about 10° C., e.g., about 4° C. A suitable cooling element is shown in FIG. 32A. Alternately, the collection chamber can be in the same section as the culture vessel, e.g., as described in Example 12.

It will be appreciated that the use of discrete, visually recognizable connected sections is but one way to achieve spatially confined environmental conditions. Use of materials with different properties, even if located so that no distinct boundaries are visible, can also be used to provide spatially distinct regions with spatially confined environmental conditions that differ from those in the culture vessel. Another approach is to direct electromagnetic radiation of an appropriate wavelength, at a particular region of a structure. The radiation can be directed towards only a portion of a structure so that the portion constitutes a spatially defined region with a spatially confined environmental condition. For example, a beam of X-rays, UV light, etc., can be directed at a portion of a structure without substantially altering the environment in adjacent portions. Such methods are used to inhibit cell growth and/or metabolism in the medium inflow channel, medium outflow channel, and/or collection chamber in certain embodiments of the invention. Cell growth and/or metabolism could also be inhibited in the collection chamber by supplying it with an appropriate inhibitory agent such as sodium azide. Furthermore, as noted above, in some embodiments of the invention spatially confined environmental conditions are achieved simply by appropriate positioning and dimensions of elements such as the culture vessel, channel(s), collection chamber, etc., without the need to utilize different materials or a sectional structure.

Another approach to preventing cell contamination of the medium reservoir and/or medium inflow channel is to include a filter having a pore size selected to prevent passage of cells through the filter at some point in the conduit and/or channel(s) that connect the medium inflow port and the the medium reservoir. For example, the medium inflow port could contain a filter blocking passage of cells out of the culture vessel. A filter such as a commercially available membrane or ceramic filter. Such filters are widely used, for example, for water purification purposes. Filter made of polycarbonate or other plastics could also be used. Preferably the filter has a pore size of 1 μm or less, 0.5 μm or less, 0.3 μm or less, 0.2 μm or less, or 0.1 μm or less. Yet another approach to preventing cell contamination of the medium reservoir and/or medium inflow channel is to introduce fresh medium into the culture vessel using a microdispenser. The microdispenser could contain a filter and/or could be located at a sufficient distance above the medium in the culture vessel as to prevent entry of cells.

Where heating and/or cooling are applied, it is preferable that the heating or cooling does not significantly affect the temperature within the culture vessel. For example, preferably the temperature within the culture vessel remains within several degrees, e.g., within ±2° C., ±1° C., within ±0.5° C., or less of the temperature that would exist in the absence of heating and/or cooling. Heat transfer modeling may be used to design structures that meet this criterion as described further in Example 10. FIG. 32B shows the results of such modeling for a realized embodiment of the microchemostat shown in FIG. 32A, indicating that local heating and cooling are predicted to have minimal if any detectable effect on the temperature within the culture vessel.

It will be appreciated that in certain embodiments the microbioreactor devices described herein generally comprise integrated systems in which culture vessel with optional sensors, associated medium inflow and outflow channels, optional valves, optional mixing elements, optional collection chambers, optional spatially distinct regions, etc., form a single structural unit, i.e., they are formed from a single block or layer of material or are formed of multiple blocks or layers of material that are physically attached so as to operate as a single unit and generally remain so throughout one or more fermentation runs. The microbioreactors may thus be referred to as “microbioreactor cassettes” or “microbioreactor chips”. Components such as optical fibers or other means for transmitting or receiving electromagnetic radiation, radiation sources, heating and cooling elements, pumps, medium and effluent reservoirs, etc. will typically be external to the single structural unit and are preferably provided with appropriate interfaces and connections thereto. Certain of these elements may also be provided as part of the single structural unit. A complete cell culture system may include a single stuctural unit comprising a culture vessel and other components such as those described above, together with one or more peripheral components.

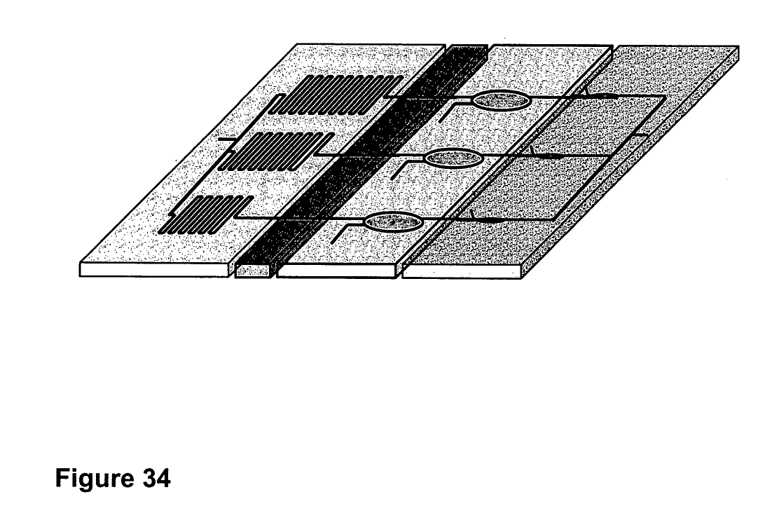

In order to operate and monitor a plurality of individual microreactors such as those described above in parallel, the apparatus described in copending patent application Ser. No. 10/816,046 may be used. Multireactor devices such as that depicted schematically in FIG. 34 are an attractive alternative. The device shown in FIG. 34 essentially replicates the individual microbioreactors described above. The device comprises a plurality of substantially identical culture vessels, each in communication with individual medium inflow and outflow channels, but makes use of a single medium channel connected to the medium reservoir, which channel divides into multiple channels to supply individual culture vessels. Similarly, medium outflow channels join to form a single channel that connects with an effluent reservoir. The device shown in FIG. 34 consists of multiple sections, which are connected via connecting elements as described above (not shown). As in the case of the individual microbioreactors, in certain embodiments of the invention the multireactor device forms a single structural unit, i.e., it is formed from a single block or layer of material or is formed of multiple blocks or layers of material that are physically attached so as to operate as a single unit and generally remain so throughout one or more fermenation runs.

FIG. 35A depicts a device similar to that in FIG. 32A, in which the section containing the culture vessel contains two additional layers. As before, a gas-permeable membrane (not shown) covers the lower body layer 90 containing the well that defines the interior of the culture vessel. Two additional body layers 92 and 94 with voids aligned with the culture vessels overlie layer 90. In certain embodiments of the invention layer 92 is made of a deformable material that helps to seal the membrane in place and provide a tight seal for channels in the upper surface of layer 90 and/or the lower surface of layer 94. In certain embodiments of the invention layer 94 is made of a rigid material. A further layer (not shown) can be added to provide an enclosed headspace for the culture vessel, which may be in communication with a channel for sampling and may contain one or more sensors (e.g., a carbon dioxide sensor). The microbioreactors are depicted within chambers 96 that provide environmental control and contain access ports for optical fibers 98. These optical fibers allow for measurements of DO (below), pH (below), and OD (above and below) as described elsewhere herein. Heating and cooling elements 100 and 102 provide temperature control in spatially distinct sections 104 and 106, respectively. FIG. 35B depicts a similar structure to that in FIG. 35A but includes an integrated heating element 108 within the single structural unit.

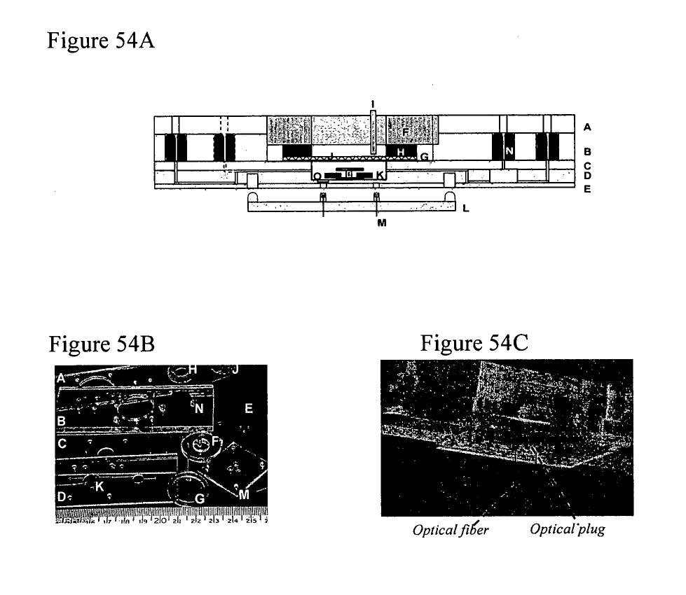

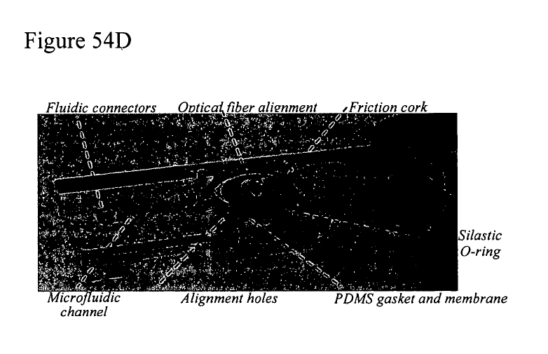

Examples 12 and 13 describe additional microbioreactors designed according to the principles designed above. Certain of these microbioreactor devices comprise one or more additional components such as microlenses, optical connectors, optical plugs, microfluidic connectors, sealing elements, functional or structural layers, etc. Exemplary embodiments are shown in FIG. 54. Optical microlenses/connectors in the integrated microbioreactor can be molded, machined, or embossed out of optically transparent materials such as glass, transparent plastics, or PDMS. A variety of embodiments are encompassed. See, e.g., references 145-146. One or more relevant dimensions of such components (e.g., diameter) are typically 1000 μm or less, often considerably less. Including such components in a microbioreactor device offers a number of advantages. For example, with optical fibers fixed in the middle of the optical plugs and a smooth, convex or hemispherical outer surface shape on the other end facing towards the optical sensors in the bioreactor, the optical plugs can effectively increase the intensity of light passed from optical fibers onto optical sensors and thus increase signal-to-noise ratio of optical measurements. In addition, in embodiments with a size comparable with the size of cavities in the microbioreactor, the optical plugs improve the alignment of optical fibers to optical sensors. It will be appreciated that any component or structure found in one or more of the microbioreactor designs described herein, including those described in the Examples and/or depicted in the Figures, may be utilized in any of the other microbioreactor designs, and the invention includes all such variations and combinations even if not explicitly set forth herein.

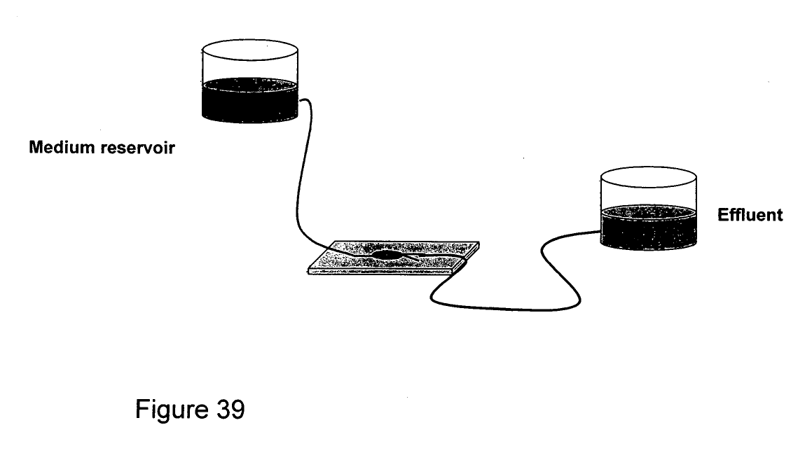

A variety of methods can be used to control medium inflow and outflow rates. Gravity-driven flow can be achieved by elevating a medium reservoir above the height of the microchemostat and maintaining the effluent reservoir below the level of the medium reservoir. By adjusting the relative heights of the medium reservoir, microchemostat, and effluent reservoir, the total rate of medium inflow to and outflow from the culture vessel can be controlled over a wide range. Since the culture vessel maintains a constant average volume, these rates will generally be equal except for insignificant contributions from evaporation. FIG. 39 illustrates the principle of passive, gravity-driven pumping under chemostat conditions.

Medium inflow and outflow can also be controlled using any of a variety of active means. Positive pressure can be exerted on the medium reservoir to cause medium to flow through the medium inflow channel into the culture vessel. Alternately, negative pressure can be exerted on the effluent or medium outflow channel. Pressure can be delivered using a constant pressure source or a motor-driven pump. Valves can be used to regulate the flow. Combinations of any of the foregoing methods can also be used.

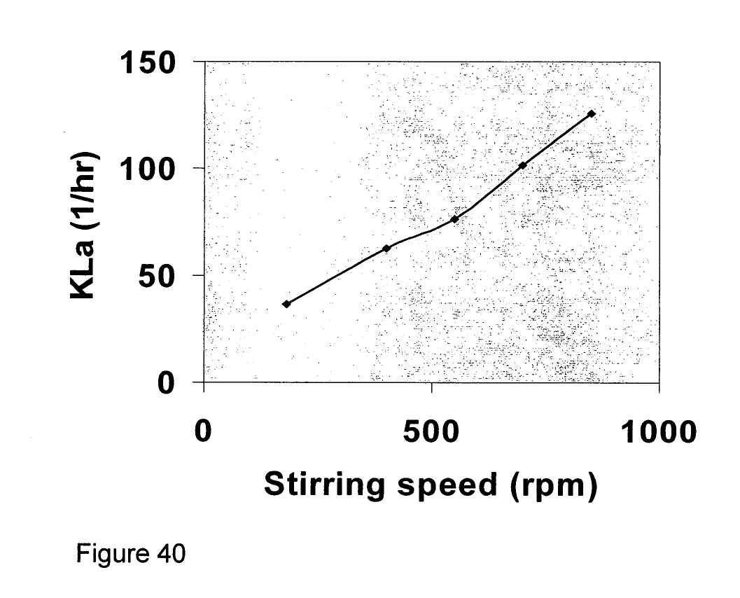

Dissolved oxygen concentration is an important parameter that can affect cell growth. As described in further detail in Example 10, chemostats can operate under conditions in which either nutrient availability or oxygen concentration limits cell growth. Oxygenation in a microchemostat can be controlled by varying the rate of active mixing. FIG. 40 shows results of an experiment in which stirring speed of a miniature magnetic stirbar was varied over a wide range and shows that the oxygen mass transfer coefficient varied in an approximately linear fashion. Higher or lower oxygen transfer rates could also be achieved by increasing or decreasing the rate of stirring (or other active mixing), respectively. Gases having higher or lower oxygen concentration than room air can be introduced into an environment control chamber housing the microchemostat, thus giving a wider range of achievable values for the oxygen transfer rate. Example 10 describes operation of the microchemostat under a variety of different conditions including oxygen-limited growth and nutrient-limited growth. Example 10 also describes changing the growth conditions during a culture period, resulting in a rapid alteration in the growth rate which is reversible when the original conditions are restored.

In certain embodiments of the invention an image is acquired from a culture during a fermentation run. A variety of suitable image sensing devices are known in the art. For example, CMOS image sensors such as the Agilent HDCS-1020 HDCS-2020 CMOS image sensors (Agilent Technologies, Palo Alto, Calif.), which include a highly sensitive active pixel photodiode array may be used. Charge coupled devices (CCDs) and intensified CCDs could also be used. Miniature cameras such as those available from Images SI, Inc., Staten Island, N.Y. are suitable. These ultra-miniature CCD cameras can be mounted on or in a chamber or a supporting component to capture information about the state of the culture during a fermentation run. The invention therefore enables the acquisition of a wide range of physiological and/or biochemical information during an ongoing fermentation run. The use of cells that express fluorescent or luminescent proteins (e.g., green fluorescent protein (GFP) and numerous related proteins and variants, luciferase, etc.) can permit monitoring and visualization of a variety of cell processes.

B. Fabrication Techniques

A wide variety of fabrication techniques may be used to construct the microreactors and microfermentors of the invention. As described in more detail in Example 1, in certain embodiments of the invention microfabrication using soft lithography is employed. This technique offers a number of advantages. For example, soft lithography allows the rapid production of microfermentors with different shapes and sizes, allowing efficient optimization of these parameters.

In certain embodiments of the invention, e.g., for purposes of large scale manufacture it may be preferable to select alternative techniques or materials. For example, in certain embodiments of the invention the microfermentor is fabricated at least in part from a polymeric material such as polystyrene, poly(carbonate), polypropylene, or polytetrafluoroethylene (TEFLON™), copolymers of aromatics and polyolefins, which can be processed using standard methods such as free-form molding, micromolding, injection molding (e.g., reaction or thermoplastic injection molding, punching, etc.), hot embossing, CNC machining, laser direct write, microelectrodischarge machining, etc. See, e.g., (78). Thermal bonding of thermoplastic materials is another useful technique that may be used. An aeration membrane can be incorporated as a structural component of the microfermentor vessel or into a vessel wall. Incorporation may occur during fabrication of the remainder of the vessel or the aeration membrane may be added later. For example, an aeration membrane may be attached using any of a variety of techniques, e.g., with adhesive, heat fusion, etc.

In certain embodiments of the invention the microfermentors and microfermentor arrays are fabricated using standard semiconductor manufacturing technology as described, for example, in (77). For example, a silicon wafer (which may be mounted on a rigid substrate such as glass or plastic) may be used to form the lower layer of the microfermentor, which can then be etched to form a well that functions as a vessel for growth of cells. Additional layer(s) of semiconductor materials such as silicon nitride may be deposited on the lower layers (e.g., by chemical vapor deposition, physical vapor deposition, and electrodeposition), with wells and channels etched into one or more of these layers. As described above, a microfermentor array including multiple wells can be formed, and the wells may be connected via channels to each other, to the edge of the wafer, or to a central receptacle, which may be used to supply nutrients, oxygen, or cells to the interior of the well and/or to remove samples.

In certain embodiments of the invention a manufacturing technique that allows substantially integrated and simultaneous fabrication of some or all of the structural components of the microfermentor (i.e., components such as bottom, top, and side walls necessary to form a vessel within which cells can be cultured) and one or more functional components (e.g., oxygen delivery means, sensors, etc.) is selected. In certain embodiments of the invention a manufacturing technique is selected that allows fabrication of some or all of the structural components of the microfermentor directly on a substrate or base. Such an approach contrasts, for example, with a manufacturing technique in which it is necessary to fabricate part of the vessel (e.g., the side walls) and then attach it to a base.

C. Materials and Surface Modification

In certain preferred embodiments of the invention biocompatible materials (i.e., materials that will not significantly inhibit or adversely affect cell viability and proliferation and/or adversely affect other biological components such as metabolites produced by the cells) are employed for those portions of the microfermentor that are in contact with cells or are used to deliver cells or other materials to the vessel. Suitable materials include silicon, silicon dioxide (e.g., glass), ceramics, plastics such as poly(carbonate)s, acrylates, polypropylenes, polyethylenes, polyolefins, or other biocompatible polymers such as silicones (for example, PDMS), fluoropolymers, etc. In addition, nonbiocompatible materials (e.g., certain metals) can be employed provided they are coated with a biocompatible material.

PDMS represents an attractive choice for microfermentor fabrication (both for the aeration membrane and as the structural material of the microfermentor itself) for a number of reasons. PDMS is highly permeable to gas, which allows sufficient oxygen to diffuse into the medium while simultaneously allowing carbon dioxide and other gases to escape. PDMS is highly hydrophobic, which minimizes water loss to evaporation. It is biocompatible, can withstand autoclaving temperatures, and is transparent to visible light.

Poly(methyl methacrylate) (PMMA) represents another attractive material for fabricating one or more layers of a multilayered microreactor structure. This material offers greater mechanical stability while also providing excellent optical transparency in the visible region, which is important for systems that include an optical sensor. In general, other materials that provide a high degree of optical transparency can also be used. Typically such materials will transmit electromagnetic radiation without substantial scattering and/or absorption over thicknesses of interest herein. For example, preferred materials may attenuate incident electromagnetic radiation by 50% or less, 75% or less, 85% or less, 90% or less, 95% or less, or 99% or less, over a path length of 1 cm, 1 mm, 0.5 mm, 0.1 mm, etc. The transparency of a material can vary in a wavelength-dependent manner. Preferred materials have a high degree of transparency over wavelengths ranging between approximately 400 and 1100 nm, preferably between approximately 400 and 800 nm.

As described above, certain microreactors of the invention comprise layers of different materials. These devices take advantage of the gas-permeable, hydrophobic, and somewhat deformable nature of PDMS and the fact that it can be readily punctured with a needle, as well as the convenience of manufacturing methods such as spin coating, while also taking advantage of the strength and rigidity of PMMA to provide good structural support.

The small sizes of the microfermentors and the other features within these systems lead to surface-to-volume ratios that are well above those in conventional macroscale operations, accentuating the importance of providing compatible interfaces for operation. Protein denaturation and non-specific adsorption provide pathways that could potentially alter the performance of the microfermentors. Thus in certain embodiments of the invention surfaces in contact with cells and/or biological components such as metabolites produced by the cells are altered in order to reduce these effects. Such surfaces may include both the interior of the microfermentor vessel and any channels, etc., that may contact either cells or other biological components such as cell products.

In certain embodiments of the invention surfaces in contact with cells or other biological components are altered in order to inhibit or promote cell adhesion. For example, in the case of bacterial cells, cellular adhesion to microfermentor surfaces is undesirable and surfaces in contact with cells may therefore be modified to reduce cell adhesion. Similarly, adhesion of cell products such as proteins may be undesirable. Adhesion may reduce the efficacy of aeration membranes and the accuracy of sensors. In addition, adhesion may contribute to denaturation of cell products and difficulty with efficient collection of such products.

To alter the adsorptive properties of the contacting surfaces of the microfermentor and any connecting microchannelled networks toward the various biological components of the system a number of different approaches may be employed. In certain embodiments of the invention the surfaces are coated with a polymer. In certain embodiments of the invention the surfaces are derivatized with self-assembling molecular films prepared from CH₃O(CH₂CH₂O)ₙ(CH₂)₁₁SiCl₃ (n=2-4) (as described in 14). These reagents produce an oriented chemisorbed monomolecular film on the surfaces of metal oxides. These films are densely packed and expose oligo(ethylene oxide) units at the surface that provide a moderately hydrophilic interface with a low interfacial energy with water. See FIG. 5. A notable feature of these films is that they are able to retard the non-specific adsorption of proteins (such as insulin, albumin, lysozyme and others) and oligonucleotides, and to greatly diminish the adsorption of cells.

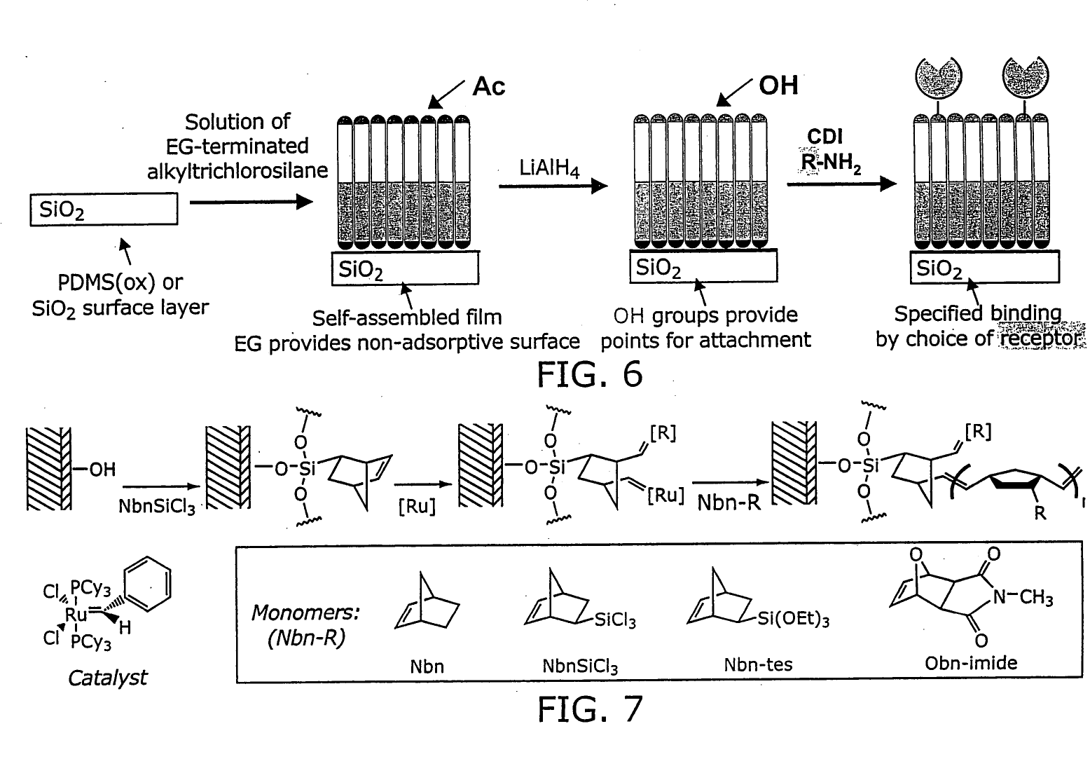

Further reductions in the adsorptive properties of cells may be achieved by the generation of more hydrophilic surfaces (i.e., surfaces with an even lower interfacial energy with water) and a greater entropic contribution against adsorption. Strategies for the production of such surfaces include the use of an acetate-terminated oligo(ethylene oxide) silanating reagent that is then deprotected on the surface to reveal hydroxyl groups or the use of reagents with longer oligo(ethylene oxide) chains. For example, the reagent CH₃CO₂(CH₂H₂O)₃(CH₂)₁₁SiCl₃ assembles to form an acetate-protected oligo(ethylene glycol) surface which, upon deprotection with LiAlH₄ produces a glycol termination. This surface presents a lower interfacial energy with water, decreases unwanted non-specific adsorption events, and offers a reactive alcohol terminus that inventors have employed to immobilize a protein through coupling using carbonyl diimidazole. See FIG. 6.

A complementary strategy for derivatizing the surfaces is the reaction between Grignard reagents (RMgBr) and a hydrogen-terminated silicon surface (15, 16). The latter is readily formed by treating a silicon surface with hydrofluoric acid. This reaction produces grafted organic chains that are connected to the surface by robust silicon-carbon bonds. This strategy offers a compatibility with basic solutions and a broader set of processing steps than do the use of silanating reagents. According to certain embodiments of the invention in which such films are employed, some amount of surface functionalization is performed during the fabrication process (particularly prior to wafer bonding steps), thereby providing possibilities for generating patterned surfaces within chips. Further, this reaction works well with porous silicon supports and offers the possibility for modifying high surface area regions within a system (9), offering a means to tailor the properties of gas-liquid interfaces used for aeration.

According to certain embodiments of the invention a surface-initiated polymerization process using ring-opening metathesis polymerization (ROMP) is used as a means to produce thicker grafted films onto surfaces (17) and to incorporate functional groups into the films. These films form at room temperature and have thicknesses that can range from 10 to 100 nm, depending on the reaction time. Briefly, the inventors used norbornenetrichlorosilane (NTCS) to assemble a monolayer coating on an oxide surface. Exposure of this primer layer sequentially to a catalyst solution and then a monomer solution resulted in formation of adherent polymer films with thicknesses of tens of nanometers. By employing NTCS as monomer in this polymerization reaction, polymeric films containing reactive functional groups were generated. The side chain trichlorosilane groups have been reacted with poly(ethylene glycol)s (PEG) to generate grafted chains of this polymer on various oxide supports. For example, in one embodiment of the invention films were treated with a 300 molecular weight PEG and then with ethylene glycol. Variants and derivatives of PEG may also be used. According to certain embodiments of the invention methoxy-capped PEGs are used.

The fact that ROMP chemistry allows a wide range of functionalities to be introduced into the films offers a synthetic flexibility and ease for accessing a broader range of surfaces, and an ability to introduce various amino acids or sugars as components within the coatings. In certain embodiments of the invention this chemistry is used to fabricate more robust coatings on the microfermentor and/or channel inner surfaces and to introduce and control a range of interfacial properties. FIG. 7 shows a schematic illustration of a surface initiated ROMP from a hydrated metal oxide surface. The surface is first derivatized to expose norbornenyl groups then treated to immobilize the [Ru] catalyst. When this surface is treated with a monomer solution, a ROMP polymer grows as a grafted film from the substrate.

According to another approach, polymers such as comb polymers (i.e., polymers that comprise polymer side chains attached to a polymer backbone) are allowed to adsorb to the surface or otherwise applied to the surface. In certain preferred embodiments of the invention the backbone of the comb polymer is selected to adsorb to the surface to be coated, and the side chains are selected to retard the adsorption of proteins and/or cells. Appropriate selection of the backbone polymer will, in general, thus depend on the particular surface to be coated. For example, in certain embodiments of the invention in which the surface is glass, variants of a polymer that includes poly(acrylic acid) as a backbone are prepared and grafted with chains of either homogenous PEG or a polymer such as poly(ethylene glycol-r-propylene glycol), containing a heterogenous mixture of molecules. The side chains may thus be identical or nonidentical.

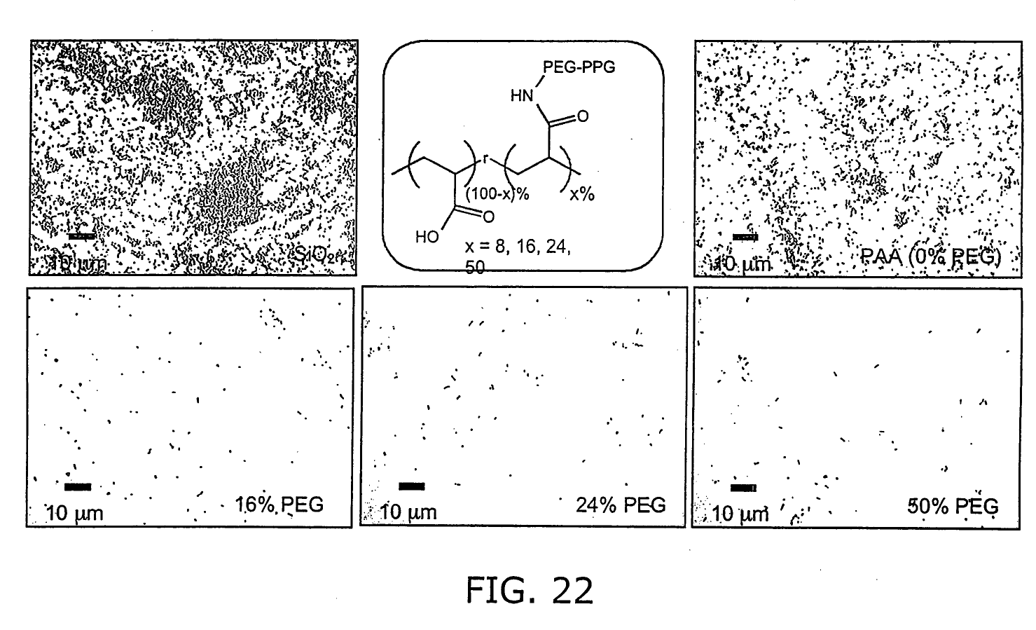

FIG. 22 shows the striking differences in cell behavior when E. coli were exposed to a bare glass surface (upper left panel) as compared with cell behavior when exposed to glass surfaces that had been treated with comb polymers having a poly(acrylic acid) backbone and a range of different PEG contents as indicated (0%, 16%, 24%, 50%). Cells were cultured in bench-scale bioreactors for 3 days in the presence of uncoated glass surfaces and glass surfaces that were coated with the various comb polymers. As is evident from FIG. 22, the presence of the comb polymers greatly decreased cell adsorption. The molecular formula of the comb polymers is presented in the upper center of the figure. The percentage number corresponds to the percent of CO₂H groups (on average) on the poly(acrylic) acid backbone that contained the PEG-PPG graft. For example, if the poly(acrylic acid) molecule comprised 100 monomer units of acrylic acid in its structure, 16% indicates that each polymer molecule contains (on average) 16 CO₂H groups with amide links to a PEG-PPG polymer chain and 84 free underivatized CO₂H groups.

The inventors have developed methods for modifying the surfaces of a variety of polymeric materials, including PDMS and PMMA, with polymers comprising PEG chains to reduce cell and protein adhesion. As described in further detail in Example 11, poly(acrylic acid) was grafted with chains of poly(ethylene glycol-r-propylene glycol) and was then adsorbed to surfaces that had been prepared so as to present an appropriate substrate for adsorption of the PAA-g-(PEG-r-PPG) copolymer. The polymer surfaces were prepared by either oxidation or reduction to produce OH groups, followed by treatment with N-6-aminohexyl)-aminopropyl trimethoxysilane (AHPTS) to form an amine-terminated self-assembled monolayer (SAM). One of ordinary skill in the art will recognize that a variety of other amine-terminated silanes could have been used, e.g., N-(2-Amino-ethyl)-3-aminopropyl-trimethoxysilane. The PAA-g-(PEG-r-PPG) polymer adsorbs to the SAM by electrostatic interactions of the ungrafted COO⁻ chains on the PAA with amines on the SAM. One of ordinary skill in the art will also recognize that a PEG or PEG-r-PPG side chains could have been grafted onto a variety of other polymers comprising a sufficient number of negatively charged moieties (e.g., COO⁻ groups) at an appropriate pH. For example, poly(methacrylic acid) (PMAA) could be used.

The invention therefore provides a method of modifying a polymeric surface with PEG comprising steps of: (a) generating OH groups on a polymeric surface; (b) assembling an amine-terminated monolayer on the surface; and (c) contacting the surface with a copolymer containing PEG and having sufficient negative charges to interact electrostatically with the amine-terminated monolayer such that stable adsorption is achieved. In certain embodiments of the invention the copolymer is a PAA-g-(PEG-r-PPG) polymer. In certain embodiments of the invention the polymer surface is a PMMA surface. As described in Example 11, modification of PDMS or PMMA surfaces with this polymer resulted in significant reduction in cell adherence. The invention provides microbioreactors in which one or more surfaces in contact with the interior of the culture vessel and/or interior of a channel is so modified.

The inventive methods for surface modification are not limited to use for a cell culture apparatus but can be used on any apparatus (e.g., any manufactured article) that comprises a suitable polymeric surface, e.g., a PMMA or poly(carbonate) surface. For example, the methods could be used for modification of surfaces of an apparatus used for downstream processing of cellular material, e.g., apparatus used to extract or purify a product. The methods could be used to modify surfaces used for packaging cells or cell products, or for packaging proteins or protein-containing solutions, e.g., therapeutic agents containing proteins.

The inventors have recognized that an advantage of using these various chemical processes for tailoring the coatings on the inner surfaces of microbioreactors is that they can be formed on the fabricated systems by simply flowing a solution of the required species through or over the device. Control over the fluidics can allow different devices (or portions of a device) to express different surface chemistries. For example, it may be desired to produce distinct regions that have a low interfacial energy with air (such as for aeration operations), that have a low interfacial energy with water (where protein and cellular adsorption is to be minimized), and that provide immobilized recognition elements for the directed adsorption of certain species (such as for sensing operations).

Self-assembly provides a powerful strategy for controlling and monitoring operations within microfabricated devices. Differences in surface reactivity (for metals vs. oxides vs. for silicon) and the abilities to direct the fluidic movements of reactants to specific regions of a device provide the ability to generate the complex patterns and progressions of surface chemistry within these microscale bioreactors for achieving the desired biochemical operation.

In contrast to bacterial cells, in the case of certain mammalian cells adhesion to a substrate promotes cell growth and may even be essential. Thus in those embodiments of the invention optimized for growth of mammalian cells, surface modifications to promote cell adhesion may be employed. In certain embodiments of the invention some surfaces or portions of surfaces are modified so as to reduce adhesion of cells, proteins, etc., while other portions are modified so as to increase adhesion. U.S. Pat. No. 6,197,575 describes various surface modifications that may be used to promote or inhibit the attachment of cells, proteins, etc., and also contains descriptions of various manufacturing techniques.

A variety of other approaches to modification of surfaces may be employed. For example, two or three dimensional stamping or contact printing may be used instead of or in conjunction with the methods described above. (See, e.g., U.S. Pat. No. 5,512,131, WO 96/29629, U.S. Pat. Nos. 6,180,239, 5,776,748). Alternatively, chemical vapor deposition, may be employed. Chemical vapor deposition allows the formation of films in the gas phase and is applicable to three dimensional devices. Among other advantages, it permits deposition of films in cavities. See, e.g., (79) and U.S. Ser. No. 09/912,166 describing chemical vapor deposition of various polymer materials (e.g., paracyclophanes) onto a variety of substrates including polyethylene, silicon, gold, stainless steel, and glass. The polymer may be a reactive polymer and/or a functionalized polymer. In certain embodiments of the invention a surface of the microfermentor vessel and/or channel(s) is coated with a polymeric material, which may incorporate a ligand. The ligand may promote or inhibit the adhesion of cells or molecules.

IV. Sensor Technology

Research in the field of bioprocess monitoring frequently aims at the rapid acquisition of accurate analytical information that can be utilized to optimize cultivation conditions, cultivation times, and product harvesting times, in order to reduce the cost and time required to establish the process. In addition, as most modern industrial bioprocesses are microbial batch or continuous-fed batch cultivations, where control of parameters is required to maintain an optimized process, on-line monitoring of the process is highly desirable. In order to optimize bioprocesses and to perform optimized bioprocesses it is desirable to be able to monitor a variety of parameters including, but not limited to, biomass and environmental variables (e.g., pH, oxygen concentration, metabolite concentration) during the course of a fermentation, for example to allow selection of fermentation conditions that maximize yield of a desired product. With conventional fermentors, this can be achieved either by in situ monitoring of the fermentor or by removing (continuously or at frequent time points) sterile samples of the contents and subjecting them to analysis.

In order to gain direct information about the concentration of single compounds in media that usually contain a complex mixture of components, analytical devices that exhibit high-selectivity for target molecules are typically required. To date, this has only been achieved by the employment of various on-line chromatographic procedures, such as liquid chromatography, gas chromatography, and mass spectrometry, and has allowed the simultaneous detection of several compounds. These types of processes, however, require expensive multi-channel devices that can take from 30-60 minutes to analyze a particular set of compounds.

In preferred embodiments of the invention at least one analytical sensor is integrated into the microfermentor. An integrated analytical sensor is a sensor that allows monitoring (which may include detection and/or measurement) of a variable of interest (e.g., an analyte) within the microfermentor vessel without the need to remove a sample of the vessel contents. The parameter of interest may be, but is not limited to: biomass, pH, dissolved oxygen, dissolved carbon dioxide, glucose, lactate, ammonia, ions such as phosphate or metal ions, any cell metabolite (which may be a protein, nucleic acid, carbohydrate, lipid, etc.), temperature. In certain embodiments of the invention the analytical sensor detects and/or measures a cell product that is to be harvested from the microfermentor or a compound that is being removed or metabolized by the cells. In certain embodiments of the invention the analytical sensor detects and/or measures a cell product that is a byproduct of metabolism, e.g., a toxic or growth-inhibitory byproduct.

In certain preferred embodiments of the invention one or more optical sensors is employed. Optical sensors have several advantages over other sensor families. They are largely immune to electromagnetic interference and cross-talk, are non-invasive, fast and work at high temperature, and are capable of continuous monitoring of an analyte even in rugged conditions such as human blood serum and fermentation broths. In addition, another desirable feature of optical sensing (e.g., using optical chemical sensors) is that it generally does not interfere with the process being measured. Furthermore, the materials are usually inexpensive, allowing their incorporation into disposable microfermentors.

In general, an optical sensor is a device that works by detecting, e.g., measuring, induced changes (i.e., changes induced by the presence of an analyte) in the absorptive, luminescent, or fluorescent properties of a medium (the chemical sensor). Generally a system employing an optical sensor includes a light source (i.e., a source of optical excitation) and a means of detecting light. Optical excitation emitted from the source excites an optical chemical sensor, which then emits luminescence or absorbs light. The luminescence emitted from the chemical sensor or the amount of light absorbed by the chemical sensor varies depending upon the concentration of the analyte. Changes in the amount of light emitted or absorbed (measured by the detector) reflect alterations in the concentration of the analyte. The chemical sensor may be supplied in any of a number of different ways. For example, in certain embodiments of the invention the chemical sensor is present in or added to the culture medium. In certain embodiments of the invention the chemical sensor is provided as a component of a sol-gel or polymer matrix or a film, which may coat at least a portion of a vessel wall or may form a structural component of the microfermentor. See, e.g., (67).

Appropriate light sources include, among others, light emitting diodes, lasers, incandescent or fluorescent lights, glow discharge, etc. Appropriate means of detecting light include spectrometers, photodetectors, charge coupled devices, diode arrays, photomultiplier tubes, etc. Optical sensing systems may also include means for collecting light and/or for transmitting it from the source or to the detector, etc. In addition, such systems may include appropriately positioned filters to filter either excitation light or emitted light. In certain embodiments of the invention fiber-optic devices are employed to transmit the light from a source and/or to a detection means. The term “fiber-optic” refers to the medium and the technology associated with the transmission of information as light impulses along a glass or plastic wire or fiber.

In addition to, or instead of, optical sensing systems, any of a wide variety of other technology platforms may be employed. Thus in certain embodiments of the invention chemical or electrochemical sensing systems can be used in conjunction with and/or integrated into the microfermentor. For example, the inventors have shown that infrared photoacoustic spectroscopy scales favorably with miniaturization and can be used as sensitive tool for a wide range of infrared active gases, including CO₂ (11).

A. Oxygen Sensing

1. Integrated Oxygen Sensor

In certain embodiments of the invention the microfermentor system includes means of monitoring dissolved oxygen (DO) within the vessel. In certain preferred embodiments of the invention an oxygen sensing means is integrated within a structural component of the microfermentor, e.g., within a microfermentor wall (i.e., not separable from the structural component without disrupting the structural integrity of the microfermentor). In certain preferred embodiments of the invention the oxygen sensing means includes an optical sensor. As described in more detail in Example 4 and in (23), oxygen can be detected via fluorescence techniques that exploit the quenching produced by oxygen on fluorophores. Suitable compounds include Ruthenium II tris(4,7-diphenyl-1,1-phenanthroline)²⁺. Its fluorescence is quenched in the presence of oxygen, and the relation between dissolved oxygen and fluorescence intensity has been shown to be nearly linear (33). In addition, this compound is sterilizable (34) and has been incorporated into both polymer (34) and sol-gel matrices (35). Such features are desirable for a fluorophore to be used in an optical sensor. Of course any of a number of other oxygen-sensitive compounds may be used. According to certain embodiments of the invention such a compound is incorporated into a structural component of the microfermentor, e.g., into an optically transparent bottom, top, or side wall. For example, as described in more detail in Example 4, the compound may be incorporated into a sol-gel that is applied to a structural component of the microfermentor (in this case a glass slide that forms the microfermentor base). Alternately, the compound may be applied to the bottom, top, and/or one or more sides of the microfermentor interior with or without a support and may be immobilized at this location. The compound may also be incorporated directly into the material from which the structural component is fabricated.

B. pH and Analyte Monitoring

In certain embodiments of the invention the microfermentor system includes means of monitoring the pH of the contents of the microfermentor. In certain embodiments of the invention the microfermentor system includes means of monitoring the presence of one or more analytes in addition to or instead of oxygen. Methods employed in the context of commercially available blood gas (pH, CO₂, O₂) sensors may be adapted for use in the microfermentor. In such sensors pH is detected by a chromophore, which changes its optical spectrum as a function of the pH. Absorption—and fluorescence-based fiber-optic sensors may be used. Carbon dioxide is detected indirectly, since its diffusion in a carbonate solution fixed on the fiber tip alters the pH, so that the carbon dioxide content can be measured by measuring the pH.

Hydrogels, cross-linked networks of hydrophilic polymers, can also be used for pH sensing. These hydrogels swell in the presence of water, and various hydrogels have been synthesized that undergo large changes in their swelling ratio depending on their environment. In addition to pH, responsive hydrogels have been developed that sense various other environmental conditions including temperature, light, electric field, pressure, the presence of carbohydrates, and the presence of antigens. pH-dependent swelling is achieved through the incorporation of weakly basic or acidic groups on the polymer backbone.

Two effects allow the quantification of variable pH-responsive hydrogel swelling. The first effect is the change in optical properties of the hydrogel on swelling. For this purpose a hydrogel membrane, containing embedded microspheres 1 μm in diameter, is synthesized. The membrane is turbid because of the difference in refractive indices between the hydrogel and the microspheres. The turbidity of the membrane decreases in an acidic medium due to the swelling of the microspheres, which lowers their refractive index and brings it closer to that of the hydrogel. The change in turbidity can be detected optically (47).

A second method of quantification involves measuring changes in the hydrogel conductivity. Conductivity changes have been found to reflect differences in ionic mobility within the hydrated gel (48, 49). This effect has been used to microfabricate a conductimetric pH sensor (50, 51). Changes in sensor resistance as large as 45% per pH unit near physiological pH have been reported. Because the sensor operation is based on changes in ion mobility, it operates best in solutions of high ionic strength.

Numerous other methods for performing sensing, e.g., optical sensing, of various analytes are known in the art. See, for example, U.S.S.N. 20020025547; U.S. Pat. Nos. 6,377,721; 6,285,807, and references therein. Other approaches to the use of fiber-optic devices and/or optical chemical sensors are found, for example, in (36-39 and 83) and references therein, all of which are herein incorporated by reference.

C. Temperature Sensing

In certain embodiments of the invention temperature control is achieved by incorporating temperature sensors and resistance heaters into the design as described, for example, in (9). As described therein, the inventors have shown in the context of a micromechanical system that it is possible to heat reaction volumes uniformly while accurately monitoring the temperature. Methods of monitoring temperature using optical chemical sensors are known in the art.

D. Monitoring Biomass

A number of techniques may be employed to detect and quantify biomass (e.g., cell density). In certain embodiments of the invention biomass is monitored using optical density. Sensing of optical density can be carried out using absorbance measurements at 600 nm, as is currently done in laboratory analysis. Absorbance measurements can be made through a transparent portion of the microfermentor vessel wall or using a waveguide. Example 4 describes one embodiment in which a light source provides light to one side of the microfermentor (in this case the bottom), and light transmitted through the microfermentor is captured at a different side (in this case the top). Appropriate light sources, detectors, and light transmission devices are described above. Equipment such as lenses, filters, beam splitters, dichroics, prisms and mirrors may be incorporated to enhance detection and accuracy. According to certain embodiments of the invention a cell that produces an easily monitored reporter enzyme, e.g., a fluorescent or luminescent protein such as green fluorescent protein (GFP) is employed.

The invention also encompasses the detection of cell metabolites including, among others, NAD(P)H (a pyridine nucleotide that is an endogenous chromophore and thus may serve as a fluorescence indicator), as an alternate or complementary means of monitoring biomass (52, 53).

According to certain embodiments of the invention one or more parameters or analytes is measured using Raman spectroscopy (80, 81). This technique may be particularly appropriate for measuring organic compounds, e.g., nutrients, cellular metabolites, etc.

E. Self-Assembling Sensors

On metal surfaces, self-assembly can be used to produce modified electrodes with chemical sensing abilities. For example, thiols will adsorb onto gold microelectrodes patterned on a silicon (oxide) substrate and selectively functionalize the electrodes and not the background substrate (18). The use of electroactive thiol reagents (specifically, a quinone-thiol and a ferrocene-thiol) has provided the ability to generate pH sensors from gold electrodes with a simple fabrication methodology (19). For example, during the microfermentor fabrication, various microelectrodes can be readily introduced strategically into its structure, and self-assembly can be used subsequently to functionalize their surfaces and produce on-board chemical sensors within the device. Present abilities allow the preparation of electrochemical sensors for pH, halide detection, glucose monitoring, and a few other species and can be expanded to provide local probes for other analytes of interest.

F. Enhancing Sensitivity of Sensors

The invention encompasses a variety of approaches to enhance the sensitivity of biosensors by using integrated optical components. One such approach includes the enhancement of the interaction path length for a fluorescent indicator emitting into a waveguide and the absorption path length in evanescent wave spectroscopy. This is realized by the use of planar waveguides in silicon/silicon dioxide. A second approach is to enhance the sensitivity of the fluorescence detection process by integrating silicon avalanche photodiodes with silicon dioxide waveguides. Recently, these avalanche photodiodes have enabled single molecule detection in aqueous flows (21).

1. Waveguide Sensors

Fiber optic sensors are only one implementation of what can generally be referred to as waveguide sensors. In general, these sensors rely on the refractive index difference between the waveguide core and the waveguide cladding to confine the light. The optical field, which is present very close to the core surface, is called the evanescent wave and can be used to probe the absorption of the surrounding medium or can be excited by fluorescence. If the cladding is stripped away and the waveguide immersed in a solution of fluorescent indicator, the only fluorescence excited by the light in the waveguide core would come from dye molecules in the sheath surrounding the exposed core. Some of that fluorescence would couple back into the waveguide and come out the ends.

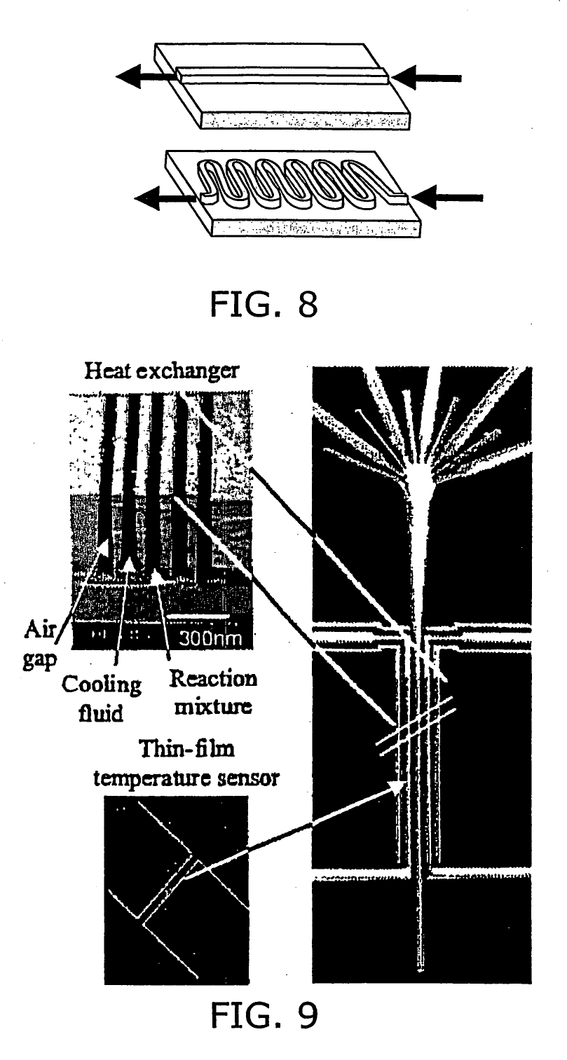

According to certain embodiments of the invention planar waveguides with rectangular cross-section are integrated on a microscale bioreactor platform. These devices allow for dramatic enhancements in interaction path length by virtue of the serpentine paths the waveguide can take through the analyte. For example, a serpentine waveguide can compress a 1 meter optical path length on a one square centimeter surface area (see FIG. 8). More importantly the total volume of this waveguide can be smaller than one nanoliter. As such, the planar waveguide can realize macroscopic optical cross-sections through microscopic analyte volumes. In certain embodiments of the invention the microscale bioreactor incorporating a waveguide sensor has an interior volume of less than or equal to 1 ml. In certain embodiments of the invention the microscale bioreactor incorporating a waveguide sensor has an interior volume of less than 200 μl. In certain preferred embodiments of the invention the working volume is between 50 μl and 100 μl inclusive. In certain preferred embodiments of the invention the working volume is between 5 μl and 50 μl, inclusive. In certain preferred embodiments of the invention the working volume is between 5 μl and 10 μl, inclusive. In certain preferred embodiments of the invention the working volume is approximately 7.5 μl or approximately 10 μl. In certain preferred embodiments of the invention the working volume is approximately 5 μl. Waveguide sensors may be fabricated using any appropriate technique. (See, e.g., U.S. Pat. No. 6,355,198 for some approaches.)

2. Single Photon Avalanche Diodes

The small volumes of the microscale bioreactors necessarily mean that analysis must be performed on small volumes of analyte. While the waveguide biosensor may have maximal interaction with the available analyte, in certain embodiments of the invention further sensitivity is realized by direct integration of photodetectors with the waveguides. Recent advances in single molecule detection within a flow cell have been made possible by the development of a single-photon avalanche diode (SPAD) with high quantum efficiency and low timing jitter. The increased fluorescence detection efficiency provided by the SPAD has enabled the detection of single chromophore molecules (23).

Silicon avalanche photodiodes with 90% quantum efficiency for wavelengths from 400-800 nm are commercially available. These devices have an internal electrical gain of 40-100 due to the avalanche process and exhibit very low noise as well as high dynamic range. Microfabricated SPAD can be easily integrated with waveguide biosensors. In this way fluorescence can be monitored from even a small number of molecules for virtually all visible and near-infrared markers used in biochemistry.

3. Optical Background in Bioreactors

A significant obstacle to coupling an optical sensor to the fermentation process is interference from the medium broth. This is due to the content of the fermentation broth, which contains cells and other opaque components. These materials absorb and scatter light, which interferes with the optical signal. The invention encompasses three approaches to deal with the complexities of bioprocess monitoring.

The first is to integrate microporous filters along the sensing surface of the waveguides. Recently, waveguide based optical sensors based on immobilization of a ruthenium complex in Nafion to monitor pH in a fermentation of Klebsiella pneumoniae have been demonstrated. Interference from the culture medium was eliminated by the addition of a black microporous filter membrane on top of the sensing film (24). These filter membranes can either be deposited after waveguide processing or they can be directly microfabricated during the sensor process.

A second approach is to employ high speed SPAD for fluorescence-lifetime spectroscopy. It has been well documented that fluorescence-lifetime methods can be successfully applied in optical sensing. These methods have considerable advantages over intensity-based methods. The fluorescence lifetime of an indicator is an intrinsic property and is virtually independent of fluctuations in light-source intensity, detector sensitivity, light throughput of the optical system, sensing layer thickness and indicator concentration (25). This implies that, in contrast to absorption methods, no reference measurement system is necessary, and, in contrast to fluorescence-intensity measurements, no compensation for variation of instrumental parameters is necessary. Lifetime-based sensors can be stable over years without any need for recalibration (26).

G. Multiple Sensing Means

Regardless of the sensing methodology employed, in certain embodiments of the invention the microscale bioreactor incorporates multiple sensors (e.g., at least 2, 3, 4, 5, or even more), thus allowing monitoring of multiple bioprocess parameters. In certain embodiments of the invention the microfermentor incorporates a sensor for monitoring oxygen. In certain embodiments of the invention the microfermentor incorporates sensors for monitoring oxygen and at least one other analyte or parameter. In certain embodiments of the invention the microfermentor incorporates sensors for monitoring oxygen and pH. In certain embodiments of the invention the microfermentor incorporates sensors for monitoring oxygen, temperature, and at least one other analyte or parameter. The sensors may be based on the same technology platform (e.g., the sensors may all be optical chemical sensors) or may be based on different technology platforms. In certain embodiments of the invention biomass and at least one additional parameter (e.g., dissolved oxygen concentration) are monitored optically. In certain embodiments of the invention the additional parameter is monitored using an optical chemical sensor. Monitoring may take place continuously, and multiple parameters may be monitored simultaneously. Where optical sensors are used it is important to avoid confounding of sensors where possible. For example, it may be important to account for the fact that absorbance readings for optical density measurements are typically made at 600 nm.

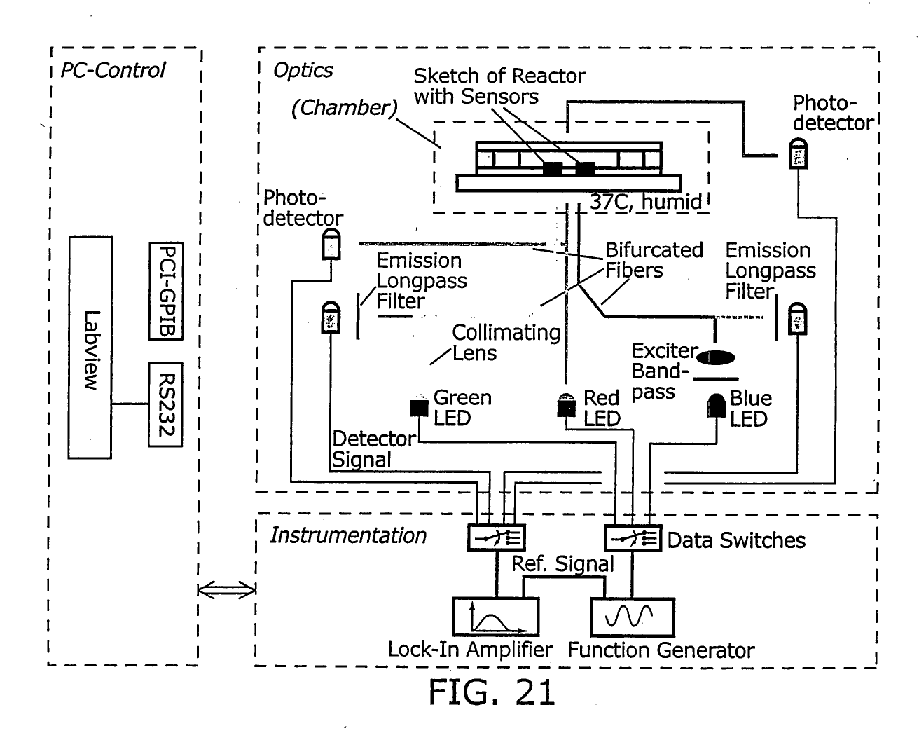

The information obtained by monitoring may be used to control and/or alter microfermentor conditions. Such monitoring and alteration may be controlled by appropriate software (e.g., the LabView system). In the case of a microfermentor array, each microfermentor may be monitored and controlled individually. FIG. 21 shows a schematic of a microfermentor integrated with optical density, dissolved oxygen, and pH sensors. As shown on FIG. 21, the microfermentor and associated optics interfaces with instrumentation and computer software to measure and/or control bioprocess parameters (see below).

V. Bioprocess Parameter Control

As described herein, in addition to monitoring of bioprocess parameters, in certain embodiments of the invention one or more of these parameters may be actively controlled and/or varied.

A. Gas Exchange

In certain embodiments of the invention oxygen delivery and/or removal of waste gases such as carbon dioxide is accomplished via a gas-permeable membrane. Preferably such a membrane is relatively impermeable to the components of the culture medium. In general, two categories of membranes that are typically used to aerate cultures—open-pore membranes (e.g. polypropylene (PP) and polytetrafluoroethylene (PTFE)), and diffusion membranes (e.g. PDMS), may be used to aerate the microfermentor.

Porous membranes consist of a polymeric matrix that contains pores from 2 nm to 10 μm in diameter. Many pore geometries exist, and together with the wide range of pore sizes give rise to several different regimes of O₂ transport, including Knudsen diffusion (narrow pores) and viscous flow (wide pores) (59). Mass transfer through a diffusion membrane (which contains molecular pores) is a function of a thermodynamic parameter, the solubility S, and a kinetic parameter, the diffusivity D. Which of these parameters dominates the mass transfer for a given polymer and penetrant depends on the nature of the interaction between the two.

Suitable materials for membranes include, for example, fluoropolymers such as the microporous membranes Teflon (e.g., Teflon AF 2400, DuPont), Goretex, cellulose acetate, porous glasses (e.g., Vycor), microporous ceramic membranes (e.g., made by sol-gel techniques), zeolite membranes, and silicones such as the diffusion membrane PDMS. Relevant permeability, solubility, and diffusivity parameters of PDMS and Teflon AF2400 are presented in Tables 1, 2, and 3 (data from 60-66).

TABLE 1
Summary of Gas Permeability, Solubility, and
Diffusivity Parameters in PDMS at 35° C.

| | P × 10¹⁰ [cm³(STP) · | S [cm³(STP)/cm³ | |
|---|---|---|---|
| Penetrant | cm/cm² · s · cmHg)] | polymer · atm] | D × 10⁵ [cm²/s] |
| O₂ | 800-933 | 0.18 | 3.4 |
| CO₂ | 3800-4570 | 1.29-1.31 | 2.2-2.64 |

TABLE 2
Summary of Water Permeability, Solubility, and
Diffusivity Parameters in PDMS at 300 K.

| | Pₗ × 10⁹ | P_{g} × 10⁵ | | | D × 10⁵ |
|---|---|---|---|---|---|
| Penetrant | [cm²/s] | [cm²/s] | Sₗ × 10³ | S_{g} | [cm²/s] |
| H₂O | 4.2-10.0 | 9.1 | 0.276-1.0 | 5.9 | 1.53-2.0 |

TABLE 3
Summary of Gas Permeability in Teflon AF 2400 at 25° C.

| Penetrant | P × 10¹⁰ [cm³(STP) · cm/cm² · s · cmHg)] |
|---|---|
| O₂ | 1600 |
| CO₂ | 3900 |

In Table 2, the solubility S is defined as the ratio of the number densities between two phases and is used to calculate the concentration at the polymer interface given the concentration in the bulk solution on both sides of the membrane. The permeability P then has units of diffusivity D, and can be thought of as an “adjusted” diffusivity. This is in contrast to the units that are normally given to permeability (Table 1), arising from the relations:

$$P = DS$$

$$and$$

$$N = \frac{D}{t} ( {C}_{1} - {C}_{2} )$$

where N is the penetrant flux through the membrane. One of ordinary skill in the art will be able to select membrane materials having appropriate diffusivities and solubilities for water, oxygen, carbon dioxide, and other penetrants.

Preferred materials are biocompatible, relatively strong, and capable of being formed into thin membranes (e.g., membranes with thicknesses on the order of the dimensions of the microfermentor. The external face of the membrane (i.e., the face not in contact with the contents of the microfermentor) is in contact with a source of oxygen that has a higher oxygen concentration than the concentration of oxygen in the microfermentor culture vessel. This oxygen source may be a gas or a liquid. In certain embodiments of the invention the source is a gas with a higher oxygen content than air. Oxygen diffuses across the membrane to provide oxygenation for the cells within the microfermentor. In certain embodiments of the invention two or more separate membranes are incorporated into the microfermentor. The external surface of the second membrane may be in contact with a gas or liquid having a lower oxygen content than the contents of the microfermentor vessel. In this manner an oxygen gradient is established across the microfermentor vessel, which facilitates oxygenation. By varying the relative oxygen concentrations with which the external faces of the membranes are in contact, it is possible to control the oxygen concentration within the microfermentor.

Although aeration membrane(s) are employed in preferred embodiments of the microfermentor system, the invention also encompasses the use of other means of providing oxygen, e.g., miniaturized magnetic stirrers, bubbling action of aeration, piezoelectric vibration, or chemical production of oxygen (in which case it is desirable to avoid the formation of toxic byproducts).

In preferred embodiments of the invention sufficient oxygen is provided to the interior of the microfermentor to support the viability and growth of bacterial cells undergoing aerobic metabolism at cell densities comparable to those employed in standard fermentation processes (e.g., approximately 10¹² cells/liter). In certain embodiments of the invention sufficient oxygen is provided to support exponential growth of bacterial cells undergoing aerobic metabolism at a range of cell concentrations, e.g., at up to approximately 10⁶ cells/l, up to approximately 10⁷ cells/l, up to approximately 10⁸ cells/l, up to approximately 10⁹ cells/l, up to approximately 10¹⁰ cells/l, up to approximately 10¹¹ cells/l, up to approximately 10¹² cells/l, or up to approximately 10¹³ cells/l. As is well known in the art, mammalian cells typically have a lower oxygen uptake rate than aerobic bacteria.

B. Climate Control

1. Temperature Control

As mentioned above, in certain embodiments of the invention temperature control is achieved by incorporating temperature sensors and resistance heaters into the design of the microfermentor. For example, the inventors have shown in the context of a micromechanical system that it is possible to heat reaction volumes uniformly while accurately monitoring the temperature (9). In addition, in certain embodiments of the invention heat exchangers for heating and cooling are incorporated into the microfermentor in a fashion analogous to that described in (10). An example of a microfabricated heat exchanger is shown in FIG. 9. The excellent heat transfer characteristics of small dimension microfabricated devices provide good thermal uniformity and small time constants. In certain embodiments of the invention the temperature is controlled to within ±2° C. In certain embodiments of the invention the temperature is controlled to within ±1° C. In certain embodiments of the invention the temperature is controlled to within ±0.1° C.

In certain embodiments of the invention temperature control is achieved by placing the microfermentor in a temperature-controlled environment, for example by placing the microfermentor in a temperature-controlled incubator or chamber as described in Example 3. Temperature control can be achieved, for example, by flowing water of a desired temperature through a chamber base.

2. Evaporation Control

In certain embodiments of the invention an appropriate humidity is maintained by placing the microfermentor in a humidity-controlled environment. For example, as described in Example 3, the microfermentor may be placed in a chamber that contains open reservoirs of water. Alternatively, humidified air may be flowed through the chamber. In preferred embodiments of the invention the chamber is sealed. Sealing the channels that lead into the microfermentor also minimizes evaporation. In addition, appropriate selection of materials for the structural components of the microfermentor (e.g., selection of hydrophobic materials) reduces evaporation.

In certain embodiments of the invention one or more membranes, one side of which in contact with the interior of the microfermentor vessel and the other side of which is in contact with humidified air or water, compensates at least in part for evaporative losses. The humidified air or water may be flowed past the membrane. As described above, various designs incorporating two vessels separated by a gas-permeable membrane may be employed.

C. pH Control

In large part because protein configuration and activity are pH dependent, cellular transport processes, reactions, and hence growth rates depend on pH. Factors such as ongoing metabolic activity may alter the pH in a culture medium. Therefore, certain embodiments of the invention include a means to control the pH. In certain embodiments of the invention pH control is achieved by providing a suitable buffer. The buffer may be provided within the culture medium. Alternately, an external buffer source may be employed, in which case the invention includes a contact between the external buffer source and the interior of the microfermentor vessel. For many bacteria, growth rates typically reach a maximum in the pH range of 6.5-7.5 (55). Typically, negligible growth occurs at a pH 1.5 to 2.0 pH units above or below the optimal pH. Many eukaryotic cells are even more sensitive to changes in pH. Accordingly, in certain embodiments of the invention the microfermentor system includes a means of controlling the pH within ±0.1 pH units of an optimum pH for cell growth. In certain embodiments of the invention the microfermentor system includes a means of controlling the pH within ±0.2 pH units of an optimum pH for cell growth. In certain embodiments of the invention the microfermentor system includes a means of controlling the pH within ±0.5 pH units of an optimum pH for cell growth. In certain embodiments of the invention the microfermentor system includes a means of controlling the pH within ±1 pH units of an optimum pH for cell growth. In certain embodiments of the invention the microfermentor system includes a means of controlling the pH within ±1.5 pH units of an optimum pH for cell growth. In certain embodiments of the invention the microfermentor system includes a means of controlling the pH within ±2 pH units of an optimum pH for cell growth. One of ordinary skill in the art will readily be able to determine the optimum pH for cell growth by reference to the scientific literature and/or by systematically culturing cells under conditions of varying pH while holding other parameters constant. The optimum pH may vary depending upon other culture parameters, e.g., nutrient supply, temperature, etc.

D. Nutrient Control

According to certain embodiments of the invention addition of nutrients, stimulants, buffers, etc., is achieved through the use of external pressure driven flows, e.g., created by pumps such as syringe pumps. See also (40) and references therein. When possible, active fluid control elements may be used. Development of such elements, e.g., valves, is currently under way in the microelectromechanical systems community and will readily be applicable in the context of the microfermentors described herein.

Alternatively, nutrients may be provided by diffusion through a membrane, e.g., from a larger reservoir, so that components are constantly renewed. Certain of the two-vessel designs described above allow for this feature.

E. Agitation

In certain embodiments of the invention agitation is used to assist in keeping the cells in suspension and prevent them from settling on the bottom of the microfermentor. Liquid within the microfermentor may be agitated by attaching the microfermentor to a moving surface (as is the case with shake flask agitation). Alternative methods of agitation may also be employed, e.g., piezoelectric effects, stirring with magnetic beads, etc.

F. Bioprocess Control in Microfermentor Arrays

The invention provides microfermentor systems comprising a plurality of microfermentors in which one or more bioprocess parameters is controlled. An exemplary embodiment is depicted in FIG. 4B. According to certain embodiments of the invention the system comprises individually addressable wells, whereby each well may receive a unique combination of inputs. According to certain embodiments of the invention each well receives the same input along one dimension and a different input along a second dimension of the array. This approach is not limited to two dimensions; rather any number of different inputs may be provided. According to certain embodiments of the invention the microfermentors are accessed by microfluidic channels. The wells may be housed in a plate or platform comprising multiple layers, one or more of which may contain channels that connect to the wells. The wells may also be addressed electronically, e.g., via wires extending therefrom. Electronic addressing may be used to control components within the wells. For example, electronic addressing may be used to control resistors within the wells to regulate temperature. In addition, data may be gathered from each well independently.

VI. Methods of Using Microfermentors and Microfermentor Arrays

A. Introduction

Fermentations are important sources of biological products used in the pharmaceutical, food, and chemical industries (54, 68-73). These products include primary and secondary metabolites, enzymes, recombinant proteins, vaccines, and the cells themselves (e.g., yeast). A hallmark of commercial fermentation processes (e.g., processes performed in production scale fermentors, by which is meant fermentors with working volumes of between 10 and 300,000 liters) has been an attempt to promote enhanced production of these industrial products through improvement of strains and/or optimization of fermentation conditions.

Strain improvement has typically been achieved through one of several procedures (mutation, genetic recombination, and genetic engineering), all of which bring about changes in the DNA sequence. These techniques are frequently used in combination with each other to reach the desired goal. Currently, improved strains are selected using an iterative cycle of three basic principles: mutation, screening, and assay. Manual screening operations are typically carried out in shake flasks or test tubes. Mutants are cultured in a primary screen, and hits are identified by measuring the total product yield using an assay such as thin layer chromatography (TLC), high-performance liquid chromatography (HPLC), or the increasingly popular enzyme-linked immunosorbent assay (ELISA). Identified hits are then taken forward and run through additional screens for confirmation.

Additionally, fermentation and cell culture can play a critical role in the elucidation of gene function in other organisms. The most common method involves the cloning and expression of a genome in a suitable host, such as E. coli or yeast, followed by fermentation in a bioreactor. The fermentation allows the identification of conditions that regulate gene expression, as well as production optimization of the protein that is then expressed. Complete genomic sequences are currently available for a wide variety of organisms including bacteria, fungi, and plants, and the amount of genomic sequence data is growing rapidly. (See, e.g., sequences available at the Web site having URL www.ncbi.nlm.nih.gov/entrez/query.fcgi?db=Genome) In particular, the recent completion of the human genome sequence provides an especially labour-intensive challenge in this area. The same issues that were identified above for the screening of improved strains are of concern here, and here again the opportunity exists for the miniaturization of culture conditions.

B. Cell Types

The microscale bioreactors of the invention may be used to culture and monitor cells of any type including microorganisms such as bacteria (e.g., eubacteria, archaebacteria), filamentous or non-filamentous fungi (e.g., yeast), protozoa, and also plant cells, insect cells, mammalian cells, etc. Bacteria may be aerobes, facultative anaerobes, or anaerobes and include, but are not limited to, members of the following genera: Escherichia, Enterobacter, Streptomyces, Azotobacter, Erwinia, Bacillus, Pseudomonas, Klebsiella, Proteus, Salmonella, Serratia, Shigella, Rhizobia, Rhodococcus, Vitreoscilla, and Paracoccus. (See the Web sites with URLs www.bacterio.cict.fr/eubacteria.html and www.bacterio.cict.fr/archaea.html for lists of bacteria that may be used.). Yeast include, but are not limited to, members of the genera: Saccharomyces, Schizosaccharomyces, Moniliella, Aureobasidium, Torulopsis, Candida, Trigonopsis, Trichosporon, Torulopsis, Zygosaccharomyces, and Yallowia. Insect cells, e.g., cells that support the growth of baculovirus such as Spodoptera frugiperda sf9 cells (see, U.S. Pat. No. 4,745,051) may be used. Such cells are particularly useful for production of recombinant proteins. Mammalian cells including, but not limited to, Chinese hamster ovary (CHO) cells, human embryonic kidney (HEK) cells, COS cells etc., may be used. See (76). In certain preferred embodiments of the methods described below the cells are of a type that is currently used in commercial bioprocesses.

The cells may be newly isolated or identified naturally occurring strains or variants, which may also be referred to as mutants. The cells may be selected, e.g., for a desirable phenotype. The cells may be genetically modified, e.g., using recombinant DNA technology. For example, cell or strain variants or mutants may be prepared by introducing appropriate nucleotide changes into the organism's DNA. The changes may include, for example, deletions, insertions, or substitutions of, nucleotides within a nucleic acid sequence of interest. The changes may also include introduction of a DNA sequence that is not naturally found in the strain or cell type. One of ordinary skill in the art will readily be able to select an appropriate method depending upon the particular cell type being modified. Methods for introducing such changes are well known in the art and include, for example, oligonucleotide-mediated mutagenesis, transposon mutagenesis, phage transduction, transformation, random mutagenesis (which may be induced by exposure to mutagenic compounds, radiation such as X-rays, UV light, etc.), PCR-mediated mutagenesis, DNA transfection, electroporation, etc.

The complete genomic sequence is available for a number of different organisms including numerous bacterial species. The availability of the genomic sequence has facilitated the construction of panels of mutants, each of which bears a loss-of-function mutation in one or more genes or open reading frames (42). In some cases the particular gene bearing the loss-of-function mutation is “tagged”, making it possible to identify a particular mutant in a mixed population.

One of ordinary skill in the art will be able to select appropriate culture media and environmental conditions for any particular cell type. Parameters such as oxygen delivery, temperature, and pH, etc., may be varied as appropriate. In addition, the microfermentor properties such as surface characteristics, vessel size, etc., may be modified depending upon the features of the particular cell type to be cultured.

B. Screening for Optimal Strains

The microscale bioreactors of the invention may be used to identify optimal organisms for performing a bioprocess. Since the microfermentors allow multiple fermentations to be performed in parallel under similar or identical conditions, they find particular use in selecting a cell type that performs optimally under such conditions, e.g., a cell type that produces a maximum amount of a desired product, a cell type that does not require a particular nutrient, etc.). The similar or identical conditions may include, but are not limited to: growth medium (carbon source, nitrogen source, precursors, and nutrients such as vitamins and minerals, salts, etc.), temperature, pH, redox potential, agitation rate, aeration rate, ionic strength, osmotic pressure, water activity, hydrostatic pressure, dissolved oxygen or carbon dioxide concentration, concentration of inducers and repressors, etc. The microfermentors are useful in screening panels of naturally occurring strains, banks of mutants, banks of genetically modified organisms, etc. Multiple different cell types or strains may be cultured in parallel under similar or identical conditions. The same cell type may be grown at a range of different cell densities. Strains, mutants or variants of particular interest include, but are not limited to, auxotrophic strains, deregulated mutants, mutants resistant to feedback inhibition, mutants resistant to repression, etc. See (68) for further discussion.

An optimum strain may be selected based on a variety of criteria. For example, an optimum strain may be, but is not limited to: a strain that produces the greatest amount of a desired product in a given time; a strain that is able to produce a desired product using a particular starting material (e.g., an inexpensive starting material); a strain which is able to grow in medium lacking particular components; a strain that is able to tolerate buildup of toxic or inhibitory metabolites in the culture; a strain that is able to tolerate a wider range of growth conditions such as pH, oxygen concentration, etc.; a strain that is able to achieve a higher cell density, etc.

C. Optimizing Bioprocess Parameters

The microscale bioreactors of the invention are useful in identifying optimal bioprocess parameters for performing a given bioprocess. Since the microfermentors allow control and/or monitoring of multiple variables, e.g., biomass, oxygen concentration, etc., they may be used to determine what values for these variables lead to optimum production of a desired metabolite or optimum removal of an undesired compound. For example, the maximum growth rate may not be the optimal growth rate for such purposes. Growing cells at less than the maximum growth rate may help minimize the accumulation of byproducts that negatively impact the growth or metabolism of the organism.

Parameters that may be varied include, but are not limited to: growth medium (carbon/energy source (e.g., glycerol, succinate, lactate, and sugars such as, e.g., glucose, lactose, sucrose, and fructose), nitrogen source, precursors, and nutrients such as vitamins and minerals, salts, etc.), temperature, pH, redox potential, agitation rate, aeration rate, ionic strength, osmotic pressure, water activity, hydrostatic pressure, dissolved oxygen or carbon dioxide concentration, concentration of inducers and repressors, etc. Any of these parameters may be varied in different ways in individual microfermentors operating in parallel, so that a time-optimal manner of varying the parameters can be identified, e.g., a manner of varying the parameters so as to optimize the process, e.g., to maximize production of a desired metabolite or maximize removal of an undesired compound. See (68) for further discussion.

The availability of a large number of microfermentors, e.g., as a microfermentor array, makes it possible to systematically vary a single parameter across a wide range of values while holding other parameters constant. Perhaps of greater significance, the availability of a large number of microfermentors makes it possible to assess the effects of simultaneously varying multiple parameters across a range of values. Appropriate mathematical techniques (which will likely be embodied in software) may be employed to determine which of these parameters is significant in terms of effects on a desired output, e.g., product level or removal of an undesired compound from the culture medium See 68 and references therein, describing use of software packages such as JMP (SAS, Cary, N.C., USA) and use of experimental designs such as Plackett-Burman screening design, fractional factorial design, response surface methodology, Box-Wilson central composite design, etc. Multiple microfermentors may be operated under each set of bioprocess parameters, which may greatly increase the reliability and statistical significance of the data.

Once one or more cell strains and/or bioprocess parameters is selected using the microscale bioreactors, scale-up (e.g., to production scale fermentors) may be performed. In performing scale-up, the skilled artisan will take into account factors such as differences in oxygenation technique between microfermentors and production scale fermentors, different geometries, different shear stresses, etc. (See 68, 74, 75).

D. Additional Applications

The microfermentors and microfermentor arrays also find use in screening compounds to determine their effects on cells. For example, they may be used to identify compounds that inhibit or reduce the growth of cells and/or exert other deleterious effects on cells (e.g., DNA damage). Screening for potential deleterious effects on cells is a necessary step in the testing and/or development of compounds for any of a wide variety of uses in which plants, animals, and/or humans will be exposed to the compound. In addition, compounds that reduce or inhibit cell viability and/or growth may be useful as pharmaceuticals, disinfectants, etc. The microfermentors and microfermentor arrays may also be used to identify compounds that increase or enhance the growth of cells, that increase the ability of the cells to produce a desired metabolite or remove an undesired product, etc.

The invention encompasses the use of the microfermentors and microfermentor arrays to determine the response of cells to a compound. A “response” includes, but is not limited to a change in a parameter such as: viability, growth rate, production of a metabolite or other biosynthetic product, biotransformation of a compound, transcription of a gene, expression of a protein, etc. In general, the methods for using the microfermentors and microfermentor arrays include culturing a cell in the presence of a compound of interest and comparing the value of a parameter of interest in the presence of the compound with the value of the parameter in the absence of the compound or in the presence of a different concentration of the compound.

The microbioreactors of the invention may be used for gene expression studies of cells (e.g., bacteria, yeast, insect cells, mammalian cells, other eukaryotic cell types) including gene expression studies in which expression of a plurality of genes is measured in parallel. DNA microarray analysis is a powerful technology used for the characterization of a wide variety of biological phenomena at the molecular level. The global determination of gene expression with DNA microarrays for example could be used to study underlying differences of cells of different types, cells responses to different environmental stimuli, gene function and transcription. Microarray technology is increasingly applied in diverse fields as diverse as drug screening, environmental testing, and clinical diagnosis.

Briefly, microarray analysis of gene expression involves obtaining a sample containing RNA, e.g., a sample of cells, and applying RNA contained in the sample (or another nucleic acid obtained by reverse transcription of the RNA) to a solid support (e.g., a cDNA or oligonucleotide microarray) on which are immobilized a plurality of probes. cDNA microarrays consist of multiple (usually thousands) of different cDNAs spotted (usually using a robotic spotting device) onto known locations on a solid support, typically a rigid support such as a glass microscope slide. The cDNAs are typically obtained by PCR amplification of plasmid library inserts using primers complementary to the vector backbone portion of the plasmid or to the gene itself for genes where sequence is known. Full length cDNAs, expressed sequence tags (ESTs), or randomly chosen cDNAs from any library of interest can be chosen. Oligonucleotide microarrays, in which oligonucleotides rather than cDNAs are employed to detect gene expression, represent an alternative to the use of cDNA microarrays (Lipshutz, R., et al., Nat Genet., 21(1 Suppl):20-4, 1999). In general, the experimental approach employed with an oligonucleotide microarray is similar to that used for cDNA microarrays. However, the shorter length of olignucleotides as compared with cDNAs means that care must be used to select oligonucleotides that hybridize specifically with transcripts whose level is to be measured.

Information regarding DNA microrarray technology and its applications may be found in Heller, M J, Annu Rev Biomed Eng., 4:129-53, 2002, and references cited therein. A variety of nucleic acid arrays have been developed and are known to those of skill in the art, including those described in: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505; EP 742 287; and EP 799 897.

In a typical microarray experiment, a microarray is hybridized with differentially labeled RNA or DNA populations derived from two different samples. Most commonly RNA (either total RNA or poly A⁺ RNA) is isolated from cells or tissues of interest and is reverse transcribed to yield cDNA. In general, one or more nucleotide residues is modified to include a label, which may be directly or indirectly detectable. Generally the label is a directly detectable label, by which is meant that it need not react with another chemical reagent or molecule in order to provide a detectable signal. RNA expression is measured by monitoring hybridization of the RNA to the probes. Rather than using RNA directly, a transcription product of the RNA, e.g., a cDNA copy reverse transcribed from the RNA may be used. The RNA and/or cDNA can be amplified, preferably in a linear manner. Amplification can be performed prior to hybridization and/or following hybridization.

In general, cDNA derived from one sample (representing, for example, a particular cell type, tissue type or growth condition) is labeled with one label (e.g., one fluor) while cDNA derived from a second sample (representing, for example, a different cell type, tissue type, or growth condition) is labeled with the second label (e.g., a second fluor). Similar amounts of labeled material from the two samples are cohybridized to the microarray. A detector capable of quantitatively detecting label intensity is used to scan the microarray. Ratios of the different intensities at various positions represent the relative concentrations of cDNA molecules that hybridized to the cDNAs represented on the microarray and thus reflect the relative expression levels of the mRNA corresponding to each cDNA/gene represented on the microarray. In addition to the “two-color” approach, methods employing a single label and methods employing multiple labels can also be used. Rather than using cDNA derived from the mRNA for hybridization to a microarray, the cDNA can be transcribed to yield complementary RNA (cRNA), which can then be hybridized to a microarray. cDNA and cRNA derived from an initial RNA sample by reverse transcription, transcription, or any combination or reverse transcription and transcription in any order and any number of times, are referred to herein as “nucleic acid transcription products” of such RNA. Labels other than fluorescent labels, e.g., biotin, enzymatic labels, etc., can also be used. For example, cRNA incorporating biotin can be hybridized to a microarray. Anti-biotin antibody with an attached fluorphore is added, and the fluorescent signal is detected. Thousands of data points are generated in a typical microarray analysis and can be processed in a variety of ways using different algorithms (e.g., hierarchical clustering) and software programs, e.g., Significance Analysis of Microarrays (SAM; Stanford University) to facilitate data analysis.

While microarray analysis is well understood in general and has found numerous applications, the techniques continue to be developed. In particular, there is an ongoing need to provide methods for performing microarray analysis on very small samples. The inventors have unexpectedly discovered that it is possible to reliably perform gene expression analysis using microarrays on samples of cells cultured in microbioreactors, including those having very small interior volumes, e.g., 200 microliters or less, 50 microliters or less, etc. As described in pending application Ser. No. 10/816,046, the inventors successfully performed microarray analysis to measure gene expression from cells cultured in a microbioreactor with a vessel having a volume of only 50 microliters. Microarray analysis was successfully performed using only 500 ng of total RNA. Purified mRNA could also have been used. The inventors have therefore both recognized the desirability of using microarray analysis for gene expression profiling of cells cultured in microbioreactors and have enabled methods for doing so. Accordingly, the invention provides a method of monitoring gene expression comprising: (i) culturing cells in a microbioreactor, wherein the microbioreactor comprises a vessel with an interior volume of 200 μl or less and means for providing oxygen to the interior of the vessel; (ii) harvesting some or all of the cells; (iii) contacting RNA obtained from the cells, or a nucleic acid transcription product of such RNA, with a microarray comprising probes for a plurality of genes under conditions such that hybridization occurs; and collecting a signal from the microarray. In various embodiments of the invention either prokaryotic (eubacteria, archaebacteria) or eukaryotic cells (e.g., yeast or other fungi, protozoa, insect, mammalian, etc.) may be used. Cells infected with an infectious agent such as a bacterium or virus can be used. In certain embodiments of the invention the cells are maintained under chemostat conditions.

The continuous increase in the public release of complete genomic sequences of microorganisms offers enormous opportunity for detailed investigations of the functioning of these organisms. Genomic expression assays provide an unprecedented ability not only to look at a single aspect of physiology, but also to see how a particular gene, regulon, or modulon interacts with other aspects of physiology. Combining high-throughput growth physiology data with high-throughput gene expression values represents a fundamental improvement over present screening technologies and would lead us to the discovery of new and/or improved microorganisms to answer medical, environmental and biological problems. Thus the invention provides methods that performing one or more gene expression analyses on cells cultured in a microbioreactor, wherein at least one bioprocess parameter is monitored during the culture period. Results of the gene expression analysis may be correlated with the bioprocess parameter data. In addition to, or instead of, monitoring a bioprocess parameter, images of the culture may be obtained. This allows correlation of features such as cell morphology (e.g., under various culture conditions) with gene expression. Cells can be modified to express fluorescent or chemiluminescent proteins, and the expression of these proteins can also be monitored during or after the culture period. Thus the invention encompasses collecting one or more optical signals during the culture period. Results from the gene expression analysis, optionally also considering results from monitoring a bioprocess parameter and/or image, can be used to select a cell strain or culture condition. Thus the invention envisions collecting gene expression profiles from cultures of multiple cell strains cultured in parallel under the same or different culture conditions (e.g., different media), and comparing the gene expression profiles. For example, upregulation of genes whose expression is indicative of cell stress may suggest that a particular condition is undesirable. A cell strain in which stress response genes are not upregulated under a given culture condition may be particularly desirable. These examples provide only an overview of the various applications of gene expression analysis in conjunction with monitoring of bioprocess parameters for improving strain selection, bioprocess parameter selection, etc.

The microbioreactors of the invention may be used for proteomic studies of cells (e.g., bacteria, yeast, insect cells, mammalian cells, other eukaryotic cell types) including studies in which expression or modification of multiple different proteins is measured in parallel. Such studies are conveniently performed using protein arrays. Such arrays generally comprise a large number (e.g., >100) protein capture agents (e.g., ligands or antibodies) or proteins bound at discrete positions on a planar support material. Samples are applied to the array and any of a variety of events (e.g., binding, phosphorylation) can be measured using methods known in the art. Protein expression, modification, and/or interaction can be assessed (133).

E. Microchemostat Applications

Chemostats offer the ability to maintain cells in culture under precisely controlled and defined environmental conditions. It is thus possible to control the physiological state of a cell culture and to change from one steady state to another while maintaining the same culture. Culturing cells under chemostat conditions is thus of great interest for purposes such as gene expression studies, proteomic studies, metabolic flux analysis (131, 132). Since it is possible to alter only a single environmental parameter at a time while maintaining the others constant, it is possible to identify and isolate the contribution of that parameter to changes in gene expression, protein modification, substrate utilization, product formation, growth rate, etc. It is also possible to determine which among many environmental parameters is the limiting factor in a process such as product formation, degradation of a pollutant, etc. Such information then allows modification of the organism, e.g., through random mutation and selection or through genetic engineering, or rational selection of alternate organisms, so as to optimize a desired pathway or minimize an undesired pathway.

Continuous fermentations under chemostat conditions offer a variety of advantages for industrial scale production of products such as enzymes, therapeutic agents, etc. For example, continuous fermentations can use smaller bioreactors than batch fermentations while producing the same amount of product per unit time. Since cells and medium are continuously removed from the culture vessel, equipment needed for cell and/or medium processing can be smaller. Use of continuous culture systems reduces “down time” between fermentation runs. Perhaps most imporantly, the physiological state of cells under chemostat conditions is more uniform than under batch, fed-batch, or semi-fed batch, resulting in more consistent and predictable yields of product. Therefore it is of great interest to identify culture conditions that are optimal for continuous fermentations.

The microchemostats of the invention allow gene expression analysis, protein analysis, metabolic flux analysis, and bioprocess optimization to be performed using cultures growing under defined and constant conditions with minimal use of media and generation of waste. This is of particular importance when using medium containing a radioactive substrate, a valuable or toxic reagent, etc., or when working with infectious agents.

The invention therefore provides a variety of methods for using the microchemostats of the invention. One such method involves culturing cells in a microchemostat and monitoring the fate of one or more substrates, e.g., identifying every product that contains one or more molecules derived from the substrate. Such methods may make use of labelled substrates. For example, according to certain inventive methods cells are cultured in a microchemostat using medium containing a radioactive molecule, and the fate of a radiolabelled molecule or atom is monitored. One or more metabolites, or all of the metabolites of the molecule, is/are identified. According to yet other inventive methods a complete mass balance analysis is performed, in which both inputs to and outputs from the system are completely accounted for. Other inventive methods include identifying one or more, or all, metabolic pathways that contribute to the biotransformation or degradation of a substrate, or identifying one or more, or all, metabolic pathways that contribute to the production of a product. It can often be impractical to carry out these studies multiple times on a large scale. Performing experiments in a microchemostat also offers the ability to switch between different conditions much more rapidly than could be achieved using a conventional larger scale reactor vessel. Still other inventive methods comprise culturing cells in a microchemostat, harvesting cells from the culture, and contacting a sample derived from the cells (e.g., a DNA, RNA, protein sample, or lysate) with a gene expression array or a protein array.

In any of the above methods, cells may be cultured under a first set of controlled cell growth conditions, during which one or more samples is obtained at a first steady state. One or more of the culture conditions, e.g., a growth limiting condition, is then changed, and further sample(s) are acquired. The culture may be allowed to reach a new steady state, and samples may be acquired under the new steady state conditions. One or more assays or measurements is performed on samples (e.g., cells, medium, or both) obtained during the first steady state period, the second steady state period, and/or the period during which the culture is undergoing a transition between steady states. A comparison is performed between results obtained under the first steady state conditions, the second steady state conditions, and/or the intervening conditions. The comparison provides useful information regarding the physiological state of the cells, utilization of a substrate, formation of a product, etc. The information may be used to modify a bioprocess parameter, to guide selection or creation of an improved strain, etc. This process may be repeated a plurality of times. Results may be used to select conditions and/or cell strains for a larger scale culture process.

Yet other applications involves assessing differences between two or more populations of cells, or assessing differences that develop in a population of cells over time relative to the starting population (e.g., evolution). For example, the rate of genetic alteration, e.g., the rate of acquisition or loss of a genetic element, the mutation rate of a gene, etc., can be measured (136). According to certain inventive methods a microchemostat is inoculated with two or more populations of cells which differ at one or more genetic locations. For example, the populations may contain different alleles of a gene, or one population may contain a gene that is lacking in the other population (either on a chromosome or episome), etc. The populations of cells may differ at a plurality of different genetic locations and may be different strains, species, etc. The cells may be inoculated at a known ratio, e.g., equal concentrations of cells can be used. The cells are cultured for a period of time, preferably under steady state conditions, following which a sample is analyzed to determine the cell type composition, the presence or absence of a particular genetic marker or mutation, etc. The method can be used to determine which of two or more strains, species, etc., is better suited to a particular environmental condition. The method can be used to determine a mutation rate. For example, the method can be used to determine the rate at which cells become resistant to a toxin such as an antibiotic. Cells can be inoculated in the presence of a toxin or antibiotic, or a toxin or antibiotic can be added at a time point following inoculation. The cells are cultured for a period of time, following which a sample is removed. The number of surviving cells is assessed.

VII. Evaluation of Microfermentors and Comparison with Conventional Fermentor Technology

In certain embodiments of the invention results in the microfermentor reliably predict results that would be obtained by scaling up a bioprocess, e.g., to the scale of a commercially available fermentor. For example, in certain embodiments of the invention a strain that is identified as an optimum strain when cultured in a microfermentor is also an optimum strain when cultured under substantially the same conditions in a conventional fermentor. In certain embodiments of the invention conditions that lead to maximum production of a biosynthetic product or metabolite or that lead to maximum biotransformation or removal of an undesired compound when cells of a particular type are cultured in a microfermentor also lead to maximum production of a biosynthetic product or metabolite or to maximum biotransformation or removal of an undesired compound when cells of the same type are cultured in a conventional fermentor, e.g., a bench-scale fermentor having a culture vessel having a volume of at least 0.5 liters, or a production scale fermentor, which may have a volume of hundreds or thousands of liters. However, it is not necessary that optimum conditions in a microfermentor correspond exactly to optimum conditions in a conventional fermentor, or that rates (e.g., rates of production or removal of a compound, rates of nutrient flux, rates of gas or heat transport, etc.) under a given set of conditions correspond exactly to rates that would be obtained under substantially identical conditions in a conventional fermentor. Rather, in certain embodiments of the invention it is sufficient if conditions and/or rates obtained when cells are grown in a microfermentor may be used to predict behavior when the process is scaled up.

For purposes of initially determining how conditions in a microscale bioreactor correspond or translate to conditions in a larger scale bioreactor, it is desirable to employ a cell type or strain that is well characterized, e.g., in terms of its physiology and behavior under different conditions. Escherichia coli represents an attractive prokaryotic cell choice for use in analyzing microscale bioreactor performance and scale-up. There is a large body of literature describing the physiology of this organism (see, e.g., 41) and its behavior under different reactor conditions. In addition, this organism is currently used in a range of commercial processes including production of small molecules and screening of gene libraries. The chemical composition of this organism is very well understood in terms of elemental composition and major biochemical fluxes. Finally, this organism has been extensively studied at the genetic level; vast collections of mutants are available with many useful properties, and the complete genomic sequence of this species has been determined. A comparable degree of information on the budding yeast Saccharomyces cerevisiae is available, making this an attractive eukaryotic cell type for use in analyzing microscale bioreactor performance and scale-up.

In a number of organisms, various promoters are known to respond to different environmental conditions such as temperature, ion concentration, oxygen concentration, etc., or to physiological insults such as DNA damage, oxidative stress, etc, by increasing or decreasing transcription from a linked gene. In order to determine whether bacteria being cultured in a microfermentor are experiencing physiological stress, and in order to compare growth properties in the microfermentor with growth properties in a larger scale fermentor, strains bearing reporter genes in which such a promoter controls expression of a reporter gene (e.g., luciferase) may be employed.

Various modifications and variations of the invention described herein will be evident to one of ordinary skill in the art and are also within the scope of the claims.

### EXAMPLES

### Example 1

### Fabrication of a Microscale Bioreactor

Poly(dimethylsiloxane) (PDMS) was selected as the microfermentor fabrication material in part because of its biocompatibility and optical transparency in the visible range. The high gas permeability of this material also allows it to be used as the material for an aeration membrane. Glass was selected as the microfermentor base for its transparency and rigidity.

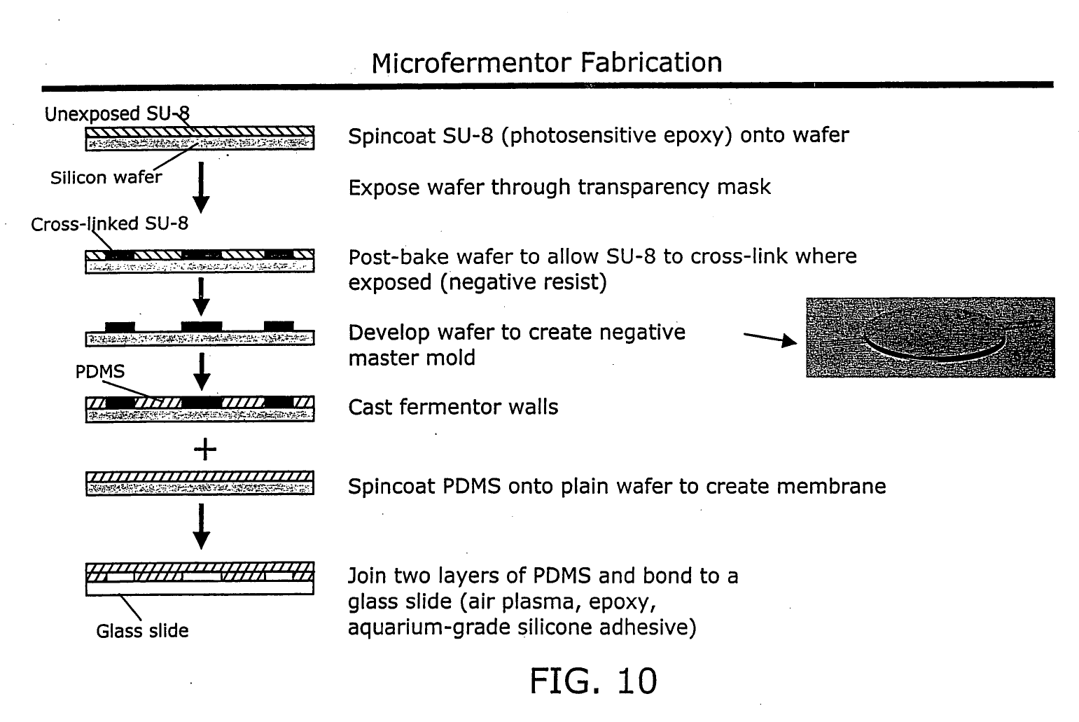

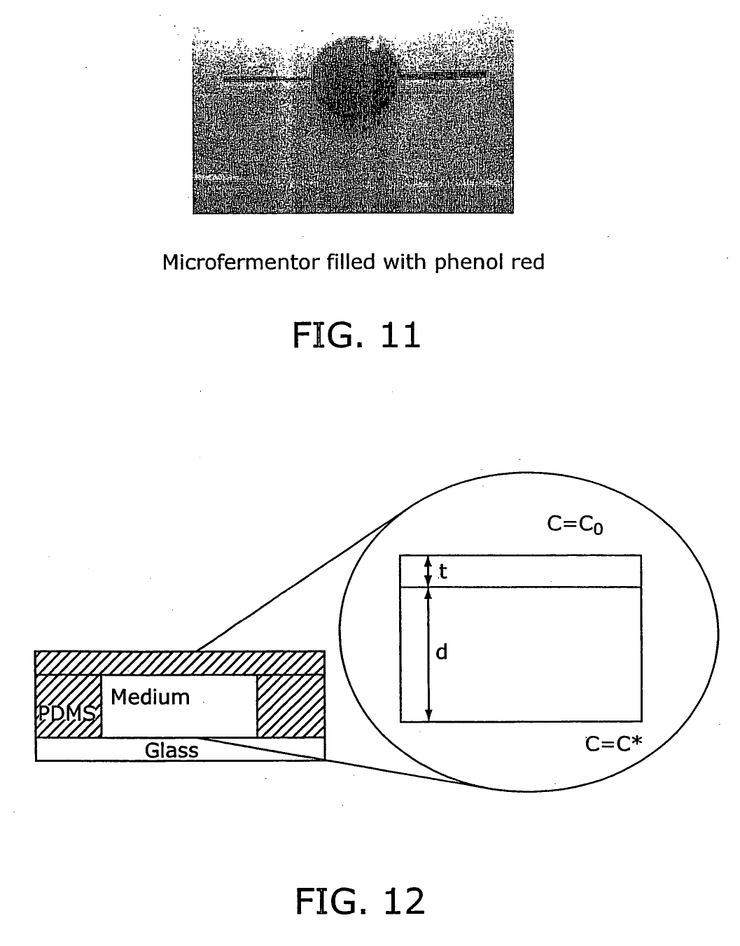

The fabrication procedure used is depicted in FIG. 10. Fabrication of the microfermentor was carried out using soft lithography as described in (58). In the first step of the fabrication process photolithography was used to fabricate a negative master out of silicon and the photo-definable epoxy SU-8. The body of the microfermentor was then cast in PDMS by squeezing the liquid polymer between the negative master and a piece of cured and passivated (silanized) PDMS. The aeration membrane was made by spin-coating the liquid polymer onto a blank wafer. The body and the membrane were subsequently joined and attached to a glass slide using epoxy or other suitable adhesives (e.g., silicone adhesives). (An air plasma seal was initially used to join the membrane to the fermentor body. However, this method appeared to result in a higher rate of evaporation of microfermentor contents, possibly due to the creation of SiO⁻ groups on the surface of the PDMS that render the surface hydrophilic. Evaporation can be avoided by, for example, maintaining the microfermentor in a humidified chamber.) A top view of a completed microfermentor filled with phenol red is shown in FIG. 11. The microfermentor has a diameter of approximately 5 mm and a depth of approximately 300 μm. The working volume of the microfermentor vessel is approximately 5 μl. Channels with a 300 μm×300 μm square cross-section extend outwards from and communicate with the vessel interior.

### Example 2

### Modeling Aeration Within a Microscale Bioreactor

Modeling of oxygen diffusion into the microfermentor was carried out using a one-dimensional resistance-in-series model of the membrane and the medium, taking oxygen consumption to be a zeroth-order reaction term (constant oxygen consumption/viable cell). For calculations at 35° C., an oxygen diffusivity in PDMS of 3.4×10⁻⁵ cm²/s and a solubility of 0.18 cm³ (STP)/cm³/atm were assumed (44). For oxygen in water a diffusivity of 2.5×10⁻⁵ cm²/s and a solubility of 7 mg/l were used (45), and it is assumed that values for culture medium would be approximately the same. A typical E. Coli oxygen uptake rate (OUR) of 30 (mmol O₂)/(gram dry cell weight/h) was assumed (46).

The models assumed a stagnant medium (no mixing). If some method of mixing is implemented, the maximum depth of the microfermentor will increase. The model assumes steady state conditions (see below for transient analysis of oxygen transport during growth). For the case where cells are spread uniformly throughout the microfermentor volume (homogeneous case), the following equations were obtained:

$${C}_{r} - {C}_{o} = {R}_{V} [ \frac{td}{{D}_{PDMS}} + \frac{{d}^{2}}{2 {D}_{{H}_{2} O}} ]$$

Where:
R_{V} is the volumetric consumption term
D is the diffusivity of oxygen in PDMS and H₂O, respectively
Cᵣ (C* in FIG. 12) is the critical oxygen concentration below which bacteria turn on anaerobic metabolic pathways (Cr=0.0082 mmol O₂/L) (from 55)

Because the solubility of oxygen in water is the main limitation (and not the permeability of the PDMS membrane) the model can be simplified by considering the medium only.

$$C ( x ) = {C}_{o} + \frac{{R}_{V} d}{D} x - \frac{{R}_{V}}{2 D} {x}^{2}$$

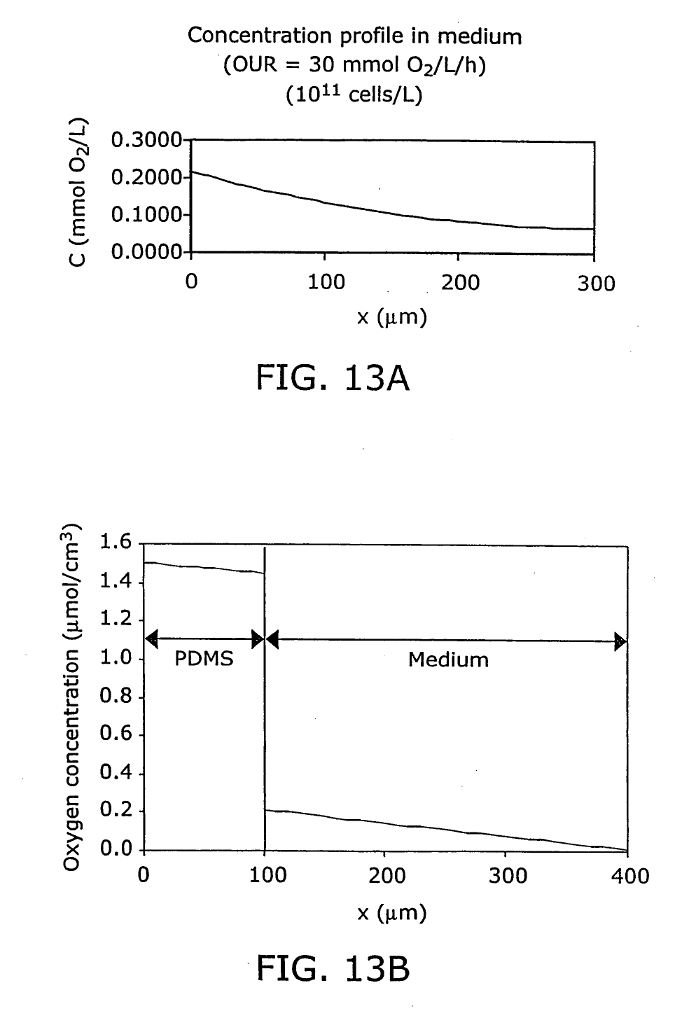

In the equation above C is the concentration at x, and x is the axis along the microfermentor depth.
The resulting plot of the oxygen concentration profile within the medium is shown in FIG. 13A.

For the case in which all cells are at the bottom of the microfermentor and consumption is heterogeneous (boundary condition), the following diffusion equation applies:

$${C}_{o} - {C}_{r} = F [ \frac{t}{{D}_{PDMS}} + \frac{d}{{D}_{{H}_{2} O}} ]$$

Here F is the flux of oxygen at the bottom of the microfermentor, corresponding to the oxygen consumption per unit area. This is converted to a volumetric term by multiplying by the ratio (A/V).

As in the homogeneous case discussed above, the maximum flux will not be realized because the limiting factor is again the solubility of oxygen in water. This can be FIG. 13B, which shows an oxygen concentration profile in the PDMS and the medium itself. The assumptions for this figure are again a cell population of approximately 10¹¹ cells/L, and a corresponding OUR of 30 mmol O₂/L/h. A membrane thickness of 100 μm, and a microfermentor depth of 300 μm were used.

As shown in FIG. 13B, the diffusion process is limited primarily by the low solubility of oxygen in water, as evidenced by the large drop-off in oxygen concentration between the membrane and the water. The diffusivity of oxygen in both phases is high enough that the slope of the profile in each phase is relatively shallow. In this case the high oxygen diffusivity combined with a high solubility in PDMS suggested that similar results would have been achieved using a thinner membrane.

The model indicates that due to the high solubility of oxygen in PDMS, the diffusivity of oxygen through the membrane could be up to an order of magnitude smaller and still provide adequate oxygenation. Therefore, any membrane with a high oxygen solubility would be compatible with the design, even if the diffusivity of the gas was 10-fold lower than that in PDMS. Alternately, if the diffusivity was as high as that in PDMS, the solubility could be more than an order of magnitude lower.

In terms of permeability:
P=DS
The permeability of PDMS is 800 Barrer (1 Barrer=10⁻¹⁰ cm³(STP)·cm/cm²·s·cm Hg) (44).

This model suggests that any membrane with an oxygen permeability >80 Barrer will work with the design, and the permeability could probably be even lower (still relatively high diffusivity, but solubility could be lower).

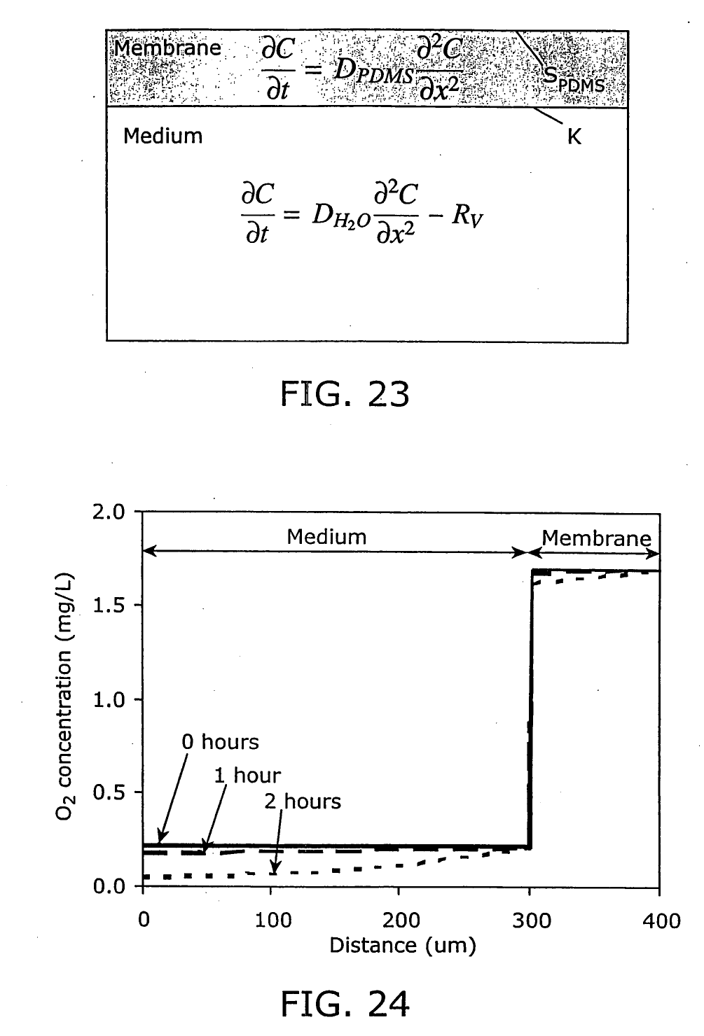

The model described above establishes the feasibility of the microfermentor design based on a steady state analysis. The design of the microfermentor can be further validated by a transient analysis of the oxygen transport during growth. FIG. 23 shows the two oxygen transport regions in the microfermentor (parameters used are listed in Table 4). The transient model assumes exponential growth (the most oxygen demanding growth phase) of homogeneously-dispersed cells, and it is based on the three equations below.

$$\frac{\partial C}{\partial t} = D \frac{{\partial }^{2} C}{\partial {x}^{2}} - {R}_{V}$$

$${R}_{V} = OxygenUptakeRate = - {Y}_{O / X} \frac{ⅆ N}{ⅆ t}$$

$$\frac{ⅆ N}{ⅆ t} = N {μ}_{max}$$

FIG. 24 shows the oxygen concentration profile across the membrane and the microbioreactor at increasing time. As in the previous example, the major resistance to mass transfer occurs in the medium rather than the membrane, a result of the low solubility of oxygen in water. It was found that a depth of 300 μm allowed sufficient oxygenation to reach a final cell number ˜10¹² cells/L. From this figure it is also apparent that a concentration gradient exists within the medium as oxygen is gradually depleted.

TABLE 4
List of parameters used in models

| Parameter | Definition | Value | Reference |
|---|---|---|---|
| S_{PDMS} | ^{†}Solubility of O₂ in PDMS | 0.18 cm³(STP)/cm³ · atm | 44 |
| D_{PDMS} | ^{†}Diffusivity of O₂ in PDMS | 3.4 × 10⁻⁵ cm²/s | 44 |
| S_{H2O} | ^{†‡}Solubility of O₂ in water | 7.36 mg/l | 45 |
| D_{H20} | ^{†‡}Diffusivity of O₂ in water | 2.5 × 10⁻⁵ cm²/s | 45 |
| K | ^{†‡}PDMS-H₂O partition coefficient | 0.129 | Calculated |
| Y_{O/X} | Yield of biomass on oxygen | 1 g_{O2} consumed/g_{DCW} (Dry | Literature |
| | | Cell Weight) produced | |
| N₀ | Initial number of cells | 3.8 × 10⁷ cells/ml | Experiment |
| t_{d} | Doubling time | 25 min | Experiment |
| μₘₐₓ | Maximum specific growth rate | 0.0278 min⁻¹ | Experiment |
| | Conversion | 2.8 × 10⁻¹³ g_{DCW}/E. coli cell | 82 |
| C* | Percent oxygen at saturation | 100% | Definition |

^{†}At 35° C., in equilibrium with 0.21 atm of oxygen
^{‡}Values for pure water were used since only 8 g/l of glucose was present in the medium
*Critical oxygen concentration = 0.0082 mmol/l (˜3.6% of air saturation) (55)

TABLE 5
List of variables used in models

| Parameter | Description |
|---|---|
| C | Concentration of oxygen |
| D | Diffusivity of O₂ in each phase |
| R_{V} | Volumetric accumulation term |
| N | Number of cells |
| μ | Specific growth rate of cells |

### Example 3

### Setup of a Microscale Bioreactor System

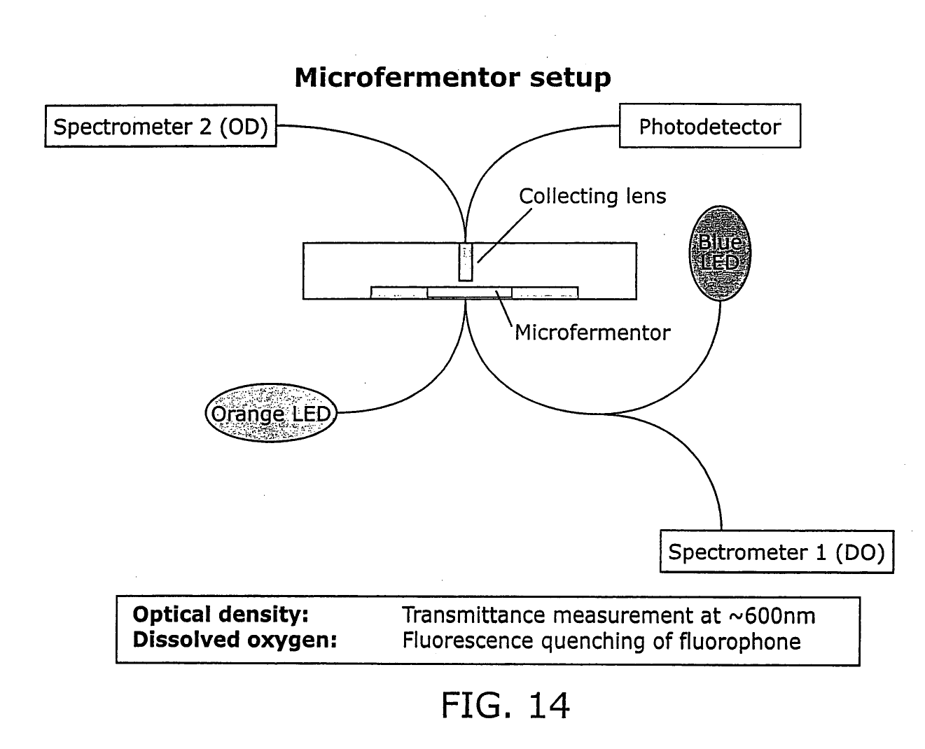

FIG. 14 shows a schematic of a microscale bioreactor system with associated optical excitation and detection sources. Optical fibers transmit light to the bottom of the fermentor. Biomass is monitored by measuring the amount of light transmitted to the collecting lens above.

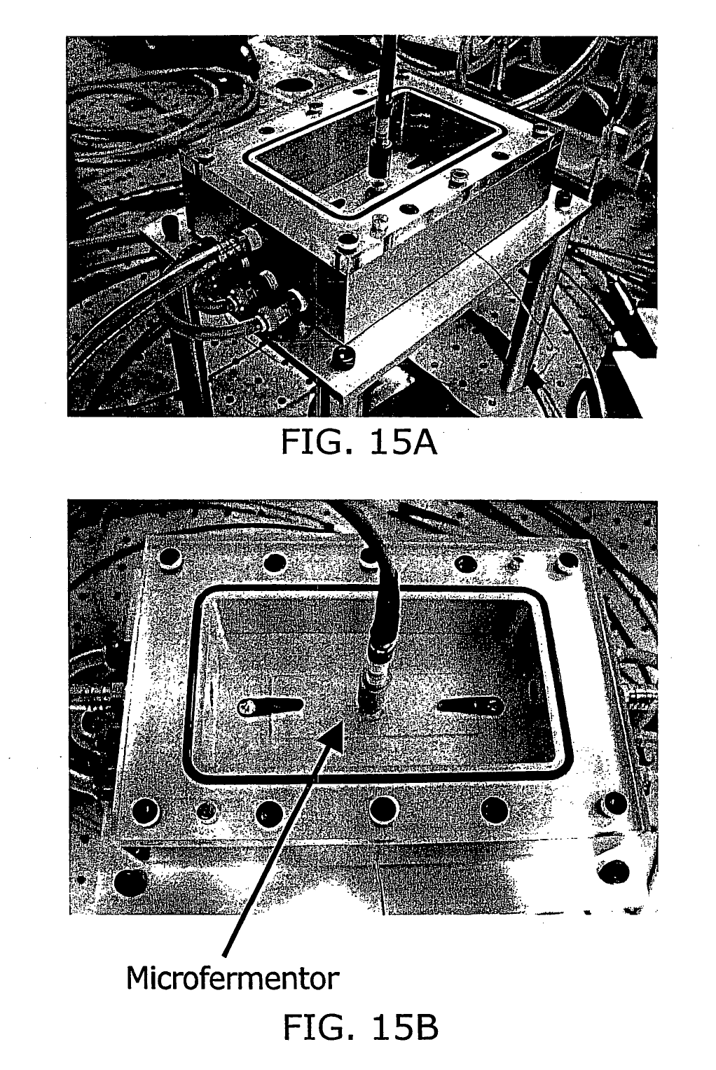

The microfermentor is placed in an enclosed chamber designed to facilitate environmental control during fermentations. The chamber is fabricated from aluminum and has a screw-on lid that can be sealed with an O-ring. FIG. 15A depicts the chamber with the microfermentor inside. FIG. 15B is a second view to more clearly show the microfermentor. (Note that the slide that forms the base of the microfermentor is transparent.) In this system, evaporation from the microfermentor is controlled by making the chamber airtight and by maintaining the air within the chamber at high humidity, e.g., 100% humidity. This is accomplished by placing open reservoirs of water beside the microfermentor within the chamber. The large volume of the chamber (˜190 cm³) as compared to the volume of the microfermentor ensures that sufficient oxygen is present to supply the needs of the growing bacteria throughout a run. Less than 1% of available oxygen is consumed by respiring bacteria during the course of a 12 hour fermentation. The chamber is maintained at a constant, desired temperature by flowing heated water from a water bath through channels within the chamber base using a heating circulator (DC-10, Thermo Haake, Karlsruhe, Germany).

Optical fibers run to the center of the chamber cover and base, above and directly below the microfermentor respectively. These fibers allow both transmissive and reflective optical measurements to be made. The fiber positioned above the microfermentor is attached to a collecting lens (F230SMA-a), ThorLabs) that increases the solid angle of capture of light emitted from the fiber below and transmitted through the microfermentor.

### Example 4

### Monitoring Bioprocess Parameters of Cells Cultured in a Microscale Bioreactor

Preparation and Inoculation of Cells

E. coli were cultured at 37° C. for 12 hours in LB medium+amp with or without addition of glucose (43). Immediately prior to introduction of the cells into the microfermentor, a 5% inoculum was introduced into fresh medium. Prior to inoculation the microfermentor was sterilized by a 60 second exposure to UV light at a wavelength of 254 nm. Inoculation of the cells was accomplished using a syringe to drive fluid through the channels and into the vessel interior. The channel holes, which self-seal to a large extent, were then further sealed using epoxy to minimize evaporation. Various epoxies and adhesives (e.g., Epoxy—ITW Performance Polymers, Part No: 46409/20845, Silicone adhesive—American Sealants, Inc., ASI #502 Silicone) have been used with no evidence of deleterious effects due to contact with cells. However, biocompatibility of the adhesive may be a consideration. Once filled, the microfermentor was placed into the chamber and secured to the base. The chamber was then closed with an airtight seal and optically sealed to prevent stray light from interfering with subsequent measurements.

Measurement of Biomass

Quantification of biomass was based on the transmission of light through the microfermentor. The light source is an orange LED with a peak wavelength of 609 nm or a helium neon (HeNe) laser with a peak wavelength of 636 nm. This light is coupled into a 600 μm optical fiber as described above. A 600 μm fiber above the microfermentor carries the transmitted light to a spectrometer (OCS-PDA, Control Development). A photodetector (PDA55, ThorLabs) is used to check for temporal power drift from the light source.

Optical density (OD) is calculated using:
OD=log₁₀(1/T)
where T=transmittance of light calculated from the intensity, I, using:
T=I_{signal}/I_{ref}

A curve for optical density as measured in a cuvette by a conventional spectrometer was obtained by diluting a sample of the fermentation medium by a factor of 10, so that it fell within into the linear portion of the spectrometer range. This value of the optical density was then used to determine the actual optical density at all other dilutions.

Measurement of Dissolved Oxygen

Fluorescence quenching of Ruthenium II tris(4,7-diphenyl-1,1-phenanthroline)²⁺ was used to measure the dissolved oxygen at the bottom of the microfermentor. The glass slide that forms the base of the microfermentor was coated with sol-gel containing this compound. These slides are available commercially (Foxy sol-gel slides, Ocean Optics). A bifurcated cable carries light at the excitation wavelength to the base of the microfermentor. The light source is USB-LS-450, Ocean Optics). Emitted light that is captured by the optical fiber is then carried back to the spectrometer (USB2000-FL, Ocean Optics), where the percent dissolved oxygen is calculated using OOISensors Software (Ocean Optics).

Results

Typical viable cell counts (based on optical density calculated from transmission data) for E. coli growing in the microfermentor in LB+amp medium without the addition of glucose indicate a cell density of approximately 4×10⁹ cells/mL (4×10¹² cells/L), comparable to that employed in large-scale fermentation processes.

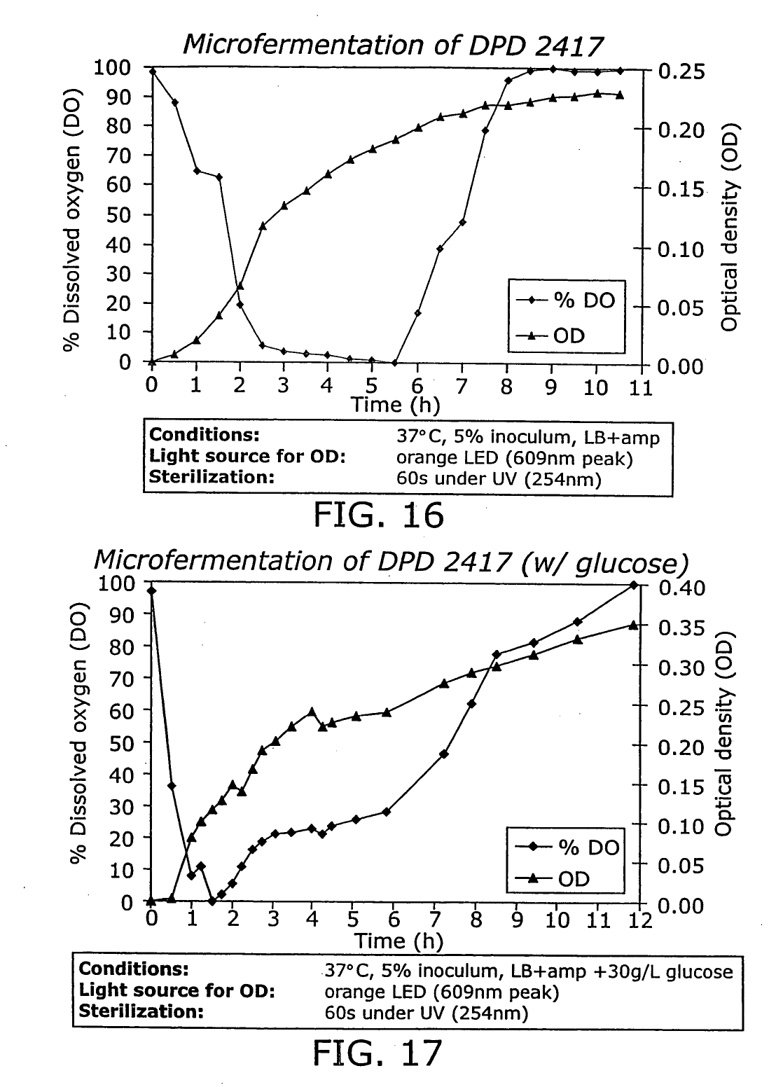

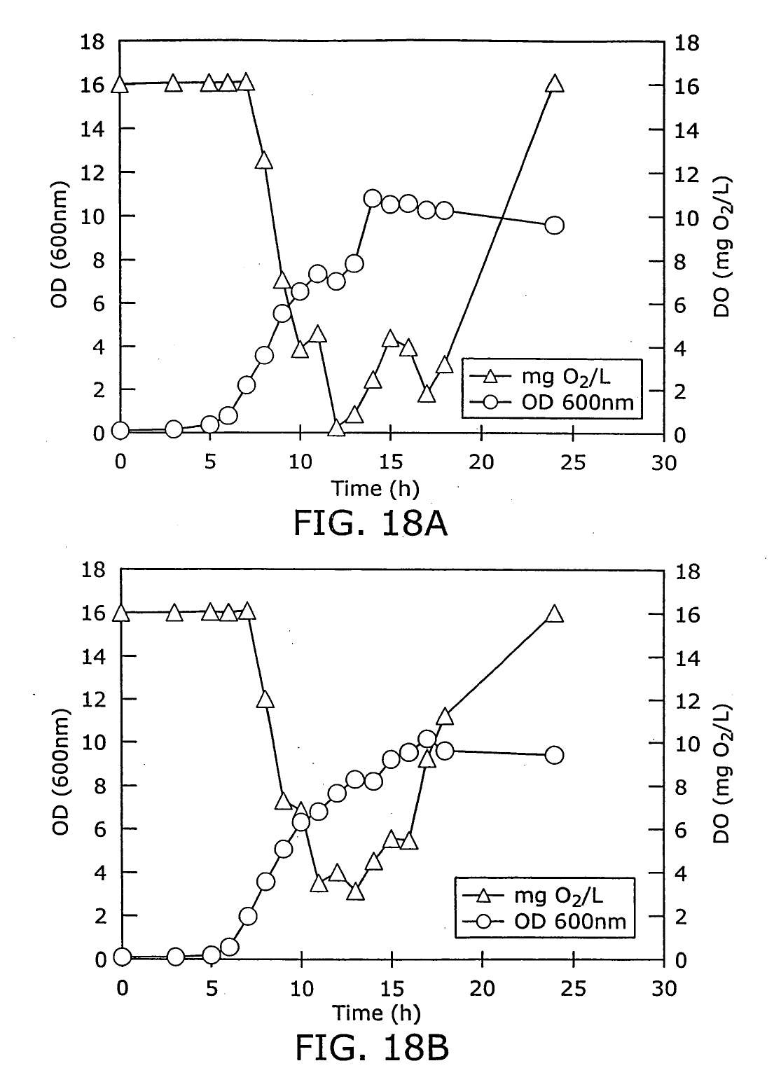

FIG. 16 shows optical density and dissolved oxygen data obtained from batch fermentation of E. coli cultured in LB+amp in a microfermentor. Oxygen was provided via the PDMS membrane, and no active stirring of the medium took place. Dissolved oxygen was measured using the Ru-based oxygen sensor. Three distinct phases of growth can be observed in FIG. 16. During the first stage, bacteria are in the exponential phase of growth and are multiplying with an apparent doubling time of 30 minutes. (The doubling time is referred to as “apparent” because in accordance with the results described above, the optical density predictably underestimates the actual biomass.) During this first stage enough oxygen is supplied by diffusion to support this rapid growth. The second stage is reached when the level of measurable oxygen in the medium drops close to zero, and oxygen is utilized by the bacteria as quickly as it diffuses into the microfermentor vessel. During this phase the bacteria switch to linear growth. Finally, the third stage shows the bacteria reaching a stationary phase. During this stage oxygen levels return to saturation. The time required to reach saturation can be predicted from the non-steady-state one dimensional diffusion equation:
∂C/∂T=D(∂²C/∂x²)
This results in an estimate on the order of minutes needed to fully reoxygenate the microfermentor to a depth of 300 μm. This time is shorter than the measured time of 2.5 hours shown in FIG. 17, but the longer reoxygenation time required is consistent with the observed accompanying increase in biomass. FIG. 17 shows a comparable curve for E. coli cultured in LB/amp+30 g/liter glucose. FIGS. 18A and 18B show fermentation of E. Coli cultured in LB/amp+30 g/liter glucose in a 0.5 liter bench scale fermentor (Sixfors) at 37 degrees, 500 RMP, aeration 2 VVM (50% O₂, 50% N₂). The growth curve and curve of oxygen concentration within the microscale bioreactor show similar trends to that obtained in the bench-scale fermentor.

### Example 5

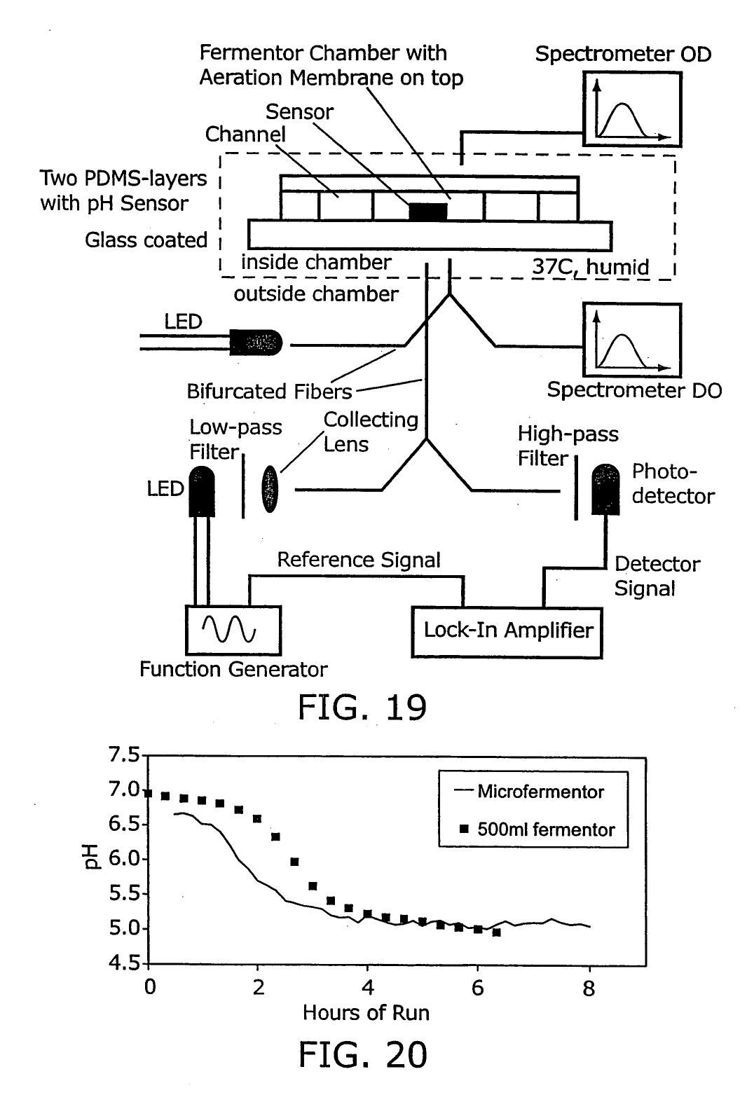

FIG. 19 shows a schematic diagram of an embodiment of the invention in which biomass, dissolved oxygen, and pH can be measured simultaneously. The microfermentor was constructed and housed in a chamber essentially as described in Examples 3 and 4. Optical density was used as a measurement of biomass. To measure dissolved oxygen, the fluorophore described above, whose fluorescence is quenched in the presence of oxygen, was excited by an LED, and the intensity of the emission was read using a spectrometer. The dissolved oxygen can also be measured using a fluorescence lifetime measurement. The pH was measured by detecting fluorescence lifetime changes in a pH sensorfoil (Presens, Regensburg, Germany) located within the microfermentor. The lifetime of the fluorescence was measured by detecting the phase-shift of the fluorescence with respect to the intensity-modulated LED using a lock-in amplifier. Bifurcated optical fibers were inserted into the bottom and top of the chamber to allow the various optical measurements to be performed.

Dissolved oxygen and biomass were measured as described in Example 4, and similar results were obtained. FIG. 20 is a graph comparing pH curves in the microfermentor and in a 0.5 L bench scale fermentor (Sixfors). The pH in the bench-scale fermentor drops after approximately 2 hours and reaches a pH of ˜5 after 6 hours. A similar trend can be observed in the microfermentor, in which the pH drops to ˜5 after 5 hours.

### Example 6

### Strain Selection Using a Microscale Bioreactor Array

Xylitol, a naturally occurring sugar alcohol, is a promising low-calorie sweetener that has lower calories than sucrose and yet exhibits comparable sweetness. It is presently as a dental caries preventive sweetener and also finds use in fluid therapy in the treatment of diabetes. For these reasons, it is expected that the demand of xylitol will increase in future. Thus the demand for xylitol is expected to increase in future.

Current industrial production of xylitol mainly relies on hydrogenation of D-xylose as disclosed in U.S. Pat. No. 4,008,285. D-Xylose used as a raw material is obtained by hydrolysis of plant materials such as trees, straws, corn cobs, oat hulls and other xylan-rich materials. However, such D-xylose, which is produced by hydrolysis of plant materials, is rather expensive and has low purity. Other production methods, utilizing D-arabitol as a starting material, are complex and involve multiple steps. Attempts to use genetic engineering to develop a microorganism with improved ability to produce xylitol have met with only limited success. Therefore, it is desirable to identify a microorganism that can produce xylitol through a single step by fermentation starting from glucose as used in the production of other saccharides and sugar alcohols.

To address this need, osmophilic microorganisms are collected from nature by enrichment culture. A medium containing 20% D-glucose, 1% yeast extract (Difco), and 0.1% urea is introduced into test tubes in an amount of 4 ml each, and sterilized at 120° C. for 20 minutes. Soil samples collected from various locations in the Cambridge, Mass. area are inoculated into the medium, and cultured at 30° C. for 4 to 7 days with shaking. When bacterial growth is observed, the cultures are plated on an agar plate having the same composition, and incubated at 30° C. for 1 to 3 days. Single colonies were isolated.

Approximately 2000 strains of osmophilic bacteria obtained as described above are cultured in individual microfermentors within a microfermentor array in a medium containing 20% (w/v) D-glucose, 0.1% urea, and 0.5% yeast extract at 30° C. for periods ranging from 12 hours to 5 days. The microfermentors have a working volume of 5 μL and are equipped with means to monitor biomass and oxygen concentration. Each microfermentor delivers oxygen to the interior of the microfermentor vessel via a PDMS aeration membrane. Each strain is introduced into 18 individual microfermentors using access channels. This allows 3 cultures to be terminated at each of 6 time points for each strain. The microfermentor array is maintained in a chamber as described in Example 3, which controls temperature and humidity. Biomass and dissolved oxygen concentration are monitored during the culture period, and data is accumulated using an appropriate software program. After an appropriate culture period (12, 24, 48, 72, 96, or 120 hours), all medium is removed from each microfermentor to be terminated at that time point and analyzed by HPLC to screen for a strain having the ability to produce xylitol.

### Example 7

### Strain Characterization and Process Parameter Optimization Using a Microscale Bioreactor Array

(1) Measurement of Acid Production and Cell Growth with Various Carbon Sources

Xylitol producing strains identified as in Example 6 are each cultured in individual microfermentors in a medium containing one of various carbon sources (1%), and presence of formed acid is determined. The following carbon sources are tested: xylose, arabinose, glucose, galactose, mannose, fructose, sorbase, sucrose, maltose, rhamnose, glycerol, mannitol, sorbitol, lactose, starch, and ethanol. The strains are pre-cultured in flasks in YPG medium at 28° C. for one day and then washed with 0.5% yeast extract solution. Since 5 strains and 16 carbon sources are tested, there is a total of 80 combinations.

Thirty microfermentors in a microfermentor array are inoculated with cells in YPC medium for each strain/carbon source combination, making a total of 2400 microfermentors. This allows 10 cultures to be terminated at each of 3 time points for each strain. (YPC is medium containing 0.5% yeast extract (Difco), and 1% of one of the various carbon sources sterilized by heating at 120° C. for 20 minutes prior to addition of the sterile carbon source. Depending on the particular pH sensor, the medium may contain a pH-sensitive dye such as bromocresol purple. The microfermentors have a working volume of 5 μl and are equipped with means to optically monitor biomass, oxygen concentration, and pH. Each microfermentor delivers oxygen to the interior of the microfermentor vessel via a PDMS aeration membrane.

The microfermentor array is maintained in a chamber as described in Example 3, which controls temperature and humidity. Biomass, dissolved oxygen concentration, and pH are monitored during the culture period, and data is accumulated using an appropriate software program. Cultures are maintained at 28° C. for 4, 5, or 6 days. After an appropriate culture period, all medium is removed from each microfermentor to be terminated at that time point and analyzed by HPLC to determine the amount of xylitol produced. The data can be used to select an appropriate strain and culture medium for a production scale fermentation process for the production of xylitol.

(2) Effect of NaCl, Acetic Acid or Ethanol Addition on Growth

Xylitol producing strains identified as in Example 6 are each cultured in individual microfermentors in YPM medium containing NaCl, ethanol, and/or acetic acid at a range of concentrations to determine the effect of these additives, singly or in combination, on growth. The xylitol producing strains and Acetobacter aceti strain NCIB 8621 as a control are pre-incubated in YPG medium (1% yeast extract (Difco), 1% peptone, sterilized by heating at 120° C. for 20 minutes, followed by addition of D-glucose to 7%) at 28° C. for one day, washed, and resuspended into medium with the one or more of the various additives at a range of concentrations. For each additive, 5 different concentrations are tested.

Thirty microfermentors are inoculated for each additive/concentration combination, allowing identical 10 cultures to be terminated at each of 3 time points. The microfermentors have a working volume of 5 μl and are equipped with means to optically monitor biomass, oxygen concentration, and pH. Each microfermentor delivers oxygen to the interior of the microfermentor vessel via a PDMS aeration membrane. The microfermentors are maintained in a chamber as described in Example 3, which controls temperature and humidity. Biomass, dissolved oxygen concentration, and pH are monitored during the culture period, and data is accumulated using an appropriate software program. Cultures are maintained at 28° C. for 4, 5, or 6 days. After an appropriate culture period, all medium is removed from each microfermentor to be terminated at that time point and analyzed by HPLC to determine the amount of xylitol produced. The data can be used to select an optimum strain and culture medium for a production scale fermentation process for the production of xylitol.

### Example 8

### Monitoring Multiple Bioprocess Parameters in a Microbioreactor

This example presents further experiments that were performed using microfermentors such as those described in Example 1. The microfermentors contained integrated sensors for on-line measurement of optical density (OD), dissolved oxygen (DO), and pH. All three parameter measurements were based on optical methods. Optical density was monitored via transmittance measurements through the microbioreactor well, while dissolved oxygen and pH were measured using fluorescence lifetime-based sensors incorporated into the body of the microbioreactor. Bacterial fermentations carried out in the microbioreactor under well-defined conditions were compared to results obtained in a 500 ml bench-scale bioreactor. It is shown that the behavior of the bacteria in the microbioreactor was similar to that in the larger bioreactor. This similarity includes growth kinetics, dissolved oxygen profile within the vessel over time, pH profile over time, final number of cells, and cell morphology. Off-line analysis of the medium to examine organic acid production and substrate utilization was performed. By changing the gaseous environmental conditions, it was demonstrated that oxygen levels within the microbioreactor can be manipulated. Furthermore, it was demonstrated that the sensitivity and reproducibility of the microbioreactor system are such that statistically significant differences in the time evolution of the OD, DO, and pH can be used to distinguish between different physiological states.

Materials and Methods

Microreactor Fabrication

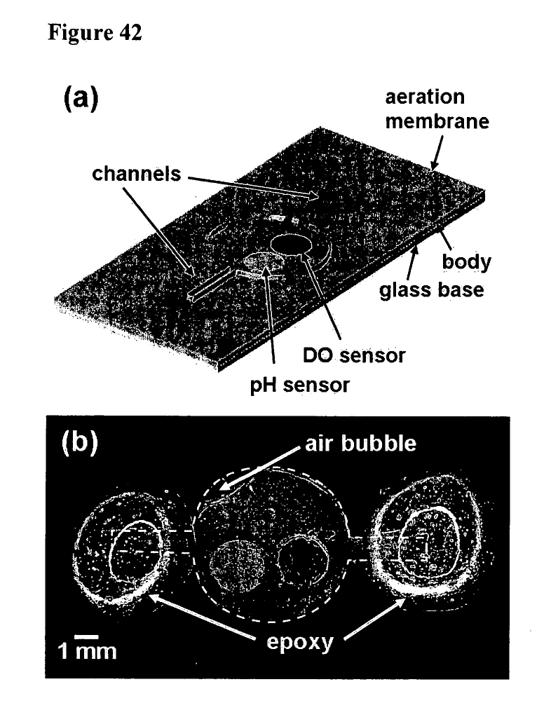

Microfermentors were fabricated out of poly(dimethylsiloxane) (PDMS) and glass essentially as described in Example 1 and elsewhere herein. PDMS was used for the body of the fermentor, the bottom layer into which the sensors were sunk, and the aeration membrane. This polymer was selected for its biocompatibility, optical transparency in the visible range, and high permeability to gases (including oxygen and carbon dioxide) as mentioned above (Merkel et al. 2000). The base support of the bioreactor was made of glass, which provided desirable rigidity as well as optical access. The typical volume of the microbioreactor was 5-50 μl, depending on the diameter used. The surface area-to-volume ratio was kept constant to ensure adequate oxygenation. The depth of the well was 300 μm, and the thickness of the aeration membrane was 100 μm. Of the experiments discussed below, those using complex medium were carried out in a volume of 5 μl, while those using defined medium were carried out in a volume of 50 μl to allow for off-line analysis of the medium. FIG. 42A shows a schematic perspective diagram of a microfermentor with integrated sensors mounted on a glass substrate.

Three PDMS layers were obtained by spincoating PDMS (Sylgard 184 Silicone Elastomer Kit, Dow Corning) onto silanized silicon wafers to the required thickness. The PDMS was then cured for two hours at 70° C., and the appropriate shapes were cut out of each layer. The bottom layer was 280 μm thick and contained two round holes into which two sensor foils were inserted, one for dissolved oxygen and one for pH as described in the following section. Each sensor was 2 mm in diameter and 150-220 μm in height. The sensors were held in place with silicone vacuum grease. Recessing the foils in this way allowed the tops to be flush with the bottom of the microbioreactor, which is especially critical for the dissolved oxygen foil as a result of the oxygen gradient that develops in the medium during fermentations (see Results). The 300 μm middle layer, which made up the body of the microbioreactor, consisted of a round opening of the desired diameter and channels for inoculation. The top layer was the 100 μm polymer aeration membrane. These layers were attached to each other and to the glass using an aquarium-grade silicone adhesive (ASI 502, American Sealants, Inc.) and allowed to cure overnight.

Optical Methods

Optical density, calculated from a transmission measurement at 600 nm, was used to monitor biomass. Light from an orange LED (Epitex L600-10V, 600 nm) was passed through the microbioreactor, collected by a collimating lens (F230SMA-A, Thorlabs), and sent to a photodetector (PDA55, Thorlabs). The optical density was calculated using the equation below, as described elsewhere herein:

$$OD = 33.33 {log}_{10} ( \frac{{I}_{reference}}{{I}_{signal}} )$$

In this equation I_{signal} is the intensity of the signal and I_{reference} is the intensity of the first measurement for a given experiment. Intensity readings were corrected for intensity fluctuations of the light source using a reference signal. The multiplication factor of 33.33 is a normalization for the pathlength of 300 μm in the microbioreactor which enables direct comparisons with results from conventional cuvettes with pathlengths of 1 cm. This adjustment is only strictly valid if the absorption and light scattering by the cell culture are in the linear region. Calibration data from the microbioreactor using known concentrations of E. coli show that the measurements are within the linear region, i.e. before saturation is reached. It is important to note that this measurement is very sensitive to both the path length and to any curvature of the PDMS aeration membrane.

Fluorescence from oxygen- and pH-sensitive dyes was selected for the measurement of dissolved oxygen (Bacon and Demas 1987; Klimant and Wolfbeis 1995; Demas et al. 1999) and pH, (Kosch et al. 1998; Lin 2000) respectively, because of the high sensitivity and specificity of this measurement (Demas and DeGraff 1991). The fluorescence of these dyes could be monitored using either fluorescence intensity or fluorescence lifetime measurements (Lakowicz 1999). There are several major advantages to using lifetime measurements. They are insensitive to background light, fluctuations of the excitation source and photodetector, changes in distance from the excitation source, bending of optical fibers, changes in medium turbidity, leaching of the indicator, and displacement of the sensing layer relative to the measurement setup.

Both dissolved oxygen and pH were monitored by phase-modulation lifetime fluorimetry using commercially available sensor foils from PreSens Precision Sensing GmbH (Regensburg, Germany). Dissolved oxygen was measured using a PSt3 sensor foil, while pH was measured using an HP2A sensor foil.

FIG. 14 shows the experimental setup. Bifurcated optical fibers (custom-made, Romack) connected to LEDs and photodetectors led into the chamber from both the top and bottom. As described above, a transmission measurement was used to calculate the optical density. The DO and pH sensors were excited with a square-wave modulated blue-green LED (NSPE590S, Nichia, 505 nm) and a blue LED (NSPB500S, Nichia, 465 nm), respectively. Exciter bandpass filters (XF 1016 and XF 1014, Omega Optical) and emission longpass filters (XF 3016 and XF 3018, Omega Optical) separated the respective excitation and emission signals and minimized cross-excitation. Data switches (8037, Electro Standards Laboratories) multiplexed the output signal and the input signal of the function generator (33120A, Agilent Technologies) and the lock-in amplifier (SR830, Stanford Research Systems), respectively. The lock-in amplifier measured and output the phase shift, which is directly related to the fluorescence lifetime, between the excitation and emission signals for the DO and pH measurement. All instruments were PC-controlled under a LabVIEW software routine, which allowed for automated and on-line measurement of the three parameters OD, DO, and pH. Readings of these parameters were taken every 10 minutes.

To determine the dissolved oxygen, the measured phase shift of the oxygen signal was related to the oxygen concentration using a modified Stern-Volmer equation (Carraway et al. 1991; Demas et al. 1995). An eleven-point calibration between 0% and 100% oxygen was carried out to confirm the validity of the equation and to calculate a Stern-Volmer constant. It was found that a better fit was obtained for low oxygen concentrations when the calibration range included in the model fit was limited to 0-21% oxygen. Therefore, data from experiments with air as the contacting gas were processed using that range, while data from experiments using pure oxygen were processed using the full range of calibration.

The measured phase shift of the pH sensor fluorescence was related to the pH by fitting to the sigmoidal Boltzmann curve (Liebsch et al. 2001). A six-point calibration was carried out between pH 4 and pH 9 using colorless buffers (VWR).

Microbioreactor Experimental Setup

Experiments were carried out in an airtight, aluminum chamber (see FIG. 14). The chamber provided a means for controlling the humidity and the composition of the gas above the microbioreactor membrane. It also provided a large thermal mass for holding the temperature at the desired set point. The interior of the chamber had an area of 11.5 cm by 6.5 cm, and a height of 2.5 cm. This volume was large compared to the volume of the microbioreactor to ensure that gaseous oxygen was in large excess compared to the oxygen consumed by the cells during a fermentation. As a result, the chamber could be sealed for the duration of a run once it had been flushed with the desired gas. Temperature was controlled with a water bath that flowed water at the desired setpoint through the chamber base. Temperature was monitored using a thermocouple.

In addition to controlling environmental parameters, the chamber provided optical isolation and optical access for the desired measurements. Optical access was from the top and bottom of the chamber, directly above and below the microbioreactor, respectively, as shown in FIG. 14.

Biological Methodology

Organism and Medium

Escherichia coli FB21591 (thiC::Tn5-pKD46, Kan^{R}) was used in all experiments and purchased from the University of Wisconsin. Stock cultures were maintained at −80° C. in 20% (vol/vol) glycerol. Prior to fermentation experiments, single colonies were prepared by streaking out the frozen cell suspension onto LB plates containing 2% (wt/vol) agar and 100 μg/ml of kanamycin. These plates were incubated overnight at 37° C. to obtain single colonies, and subsequently stored in the refrigerator at 4° C. for up to a week or used immediately to inoculate precultures.

Luria-Bertani medium was composed of 10 g/l tryptone (Difco Laboratories), 5 g/l yeast extract (Difco Laboratories), and 5 g/l NaCl. The solution was autoclaved for 40 minutes at 120° C. and 150 kPa. The LB medium was supplemented with 10 g/l glucose (Mallinckrodt), 100 mM MES buffer at pH 6.9 (2-(N-Morpholino)-ethanesulfonic acid)) (Sigma), and 100 μg/ml of kanamycin (Sigma). The glucose stock solution was autoclaved for 20 minutes at 120° C. and 150 kPa, and the MES and kanamycin stock solutions were filtered through 0.2 μm filters (Millipore).

The defined medium had the following composition: K₂HPO₄ [60 mM], NaH₂PO₄ [35 mM], (NH₄)₂SO₄ [15 mM], NH₄Cl [70 mM], MgSO₄.7H₂O [0.8 mM], Ca(NO₃)₂.4H₂O [0.06 mM], FeCl₃ [20 mM], MES [100 mM], glucose [10 g/l], thiamine [100 μM], kanamycin [100 μg/ml], (NH₄)₆Mo₇O₂₄.4H₂O [0.003 μM], H₃BO₃ [0.4 μM], CuSO₄.5H₂O [0.01 μM], MnCl₂.4H₂O [0.08 μM], ZnSO₄.7H₂O [0.01 μM]. Glucose, MES, kanamycin, and thiamine were added to the medium as stock solutions.

Precultures

For experiments using LB medium, 5 ml of sterile medium were transferred into test tubes and each was inoculated with a single colony of E. coli FB21591 from a LB-kanamycin agar plate. These cultures were incubated on a roller at 60 rpms and 37° C. Samples were removed periodically and measured for optical density (600 nm). When the optical density of the cultures reached OD=1±0.1, medium was removed from each test tube and transferred to a 500 ml baffled shake flask containing 30 ml of fresh medium to a starting optical density of 0.05. The inoculated shake flasks were incubated on shakers (150-200 rpm) at 37° C. Samples were withdrawn periodically until the optical density within the flasks reached OD=1. At this point the culture was used to inoculate either the bench-scale bioreactors or a microbioreactor.

Precultures for experiments using defined medium were carried out as above, except that the shake flasks into which the cultures from the test tubes were transferred contained defined medium.

Bench-Scale Bioreactor

Batch cultures were grown in 500 ml SixFors bioreactors (Infors, Switzerland) with a starting medium volume of 450 ml. Dissolved oxygen probes (405 DPAS-SC-K8S/200, Mettler Toledo) were calibrated with nitrogen gas (0% DO) and air (100% DO) prior to each run. pH probes (InPro 6100/220/S/N, Mettler Toledo) were calibrated with buffer at pH 7.0 and 4.0 (VWR).

The bioreactors were inoculated to a starting optical density of 0.05. The aeration rate of gas was set to 1 VVM (volume of gas per volume of medium per minute) and the impeller speed was set to 500 rpm. This combination of stirring and sparging was selected to match the estimated k_{L}a of the microbioreactor. The k_{L}a was measured using the well-known method of “dynamic gassing out” (Van Suijdam et al. 1978). The temperature of the vessels was maintained at 37° C. for all fermentations. Dissolved oxygen and pH were not controlled, so as to simulate the batch microbioreactor. The time courses of temperature, dissolved oxygen, and pH were recorded every 10 minutes throughout all fermentations. Biomass was monitored by removing samples from the bioreactor at defined time intervals and measuring the optical density at 600 nm on a spectrophotometer (Spectronic 20 Genesys, Spectronic Instruments).

Microbioreactor

Inoculation of the medium for the microbioreactor was carried out outside of the bioreactor. Ten milliliters of fresh medium were transferred to a Falcon conical tube, and to this was added the preculture medium from a shake flask for a starting optical density of 0.05. This inoculated medium was then introduced into the microbioreactor by injecting the liquid via channels (FIGS. 42A and 42B).

Sterility was maintained through the use of the antibiotic kanamycin in the medium. Other methods of sterilizing, such as autoclaving and UV radiation, were not feasible due to the incompatibility of either the DO sensor or the pH sensor with each of these methods. Gamma radiation was tested as an alternative technique. Ethanol could also be used as a means of sterilization. However, for the present studies we found that using a fast-growing, antibiotic-resistant strain was sufficient for preventing contamination.

To ensure the flatness of the PDMS membrane, excess liquid was squeezed out of the chamber by applying a uniformly distributed pressure from the top. A bulge in the membrane would change the path length for the calculation of optical density, as well as change the distance over which diffusion of oxygen occurred, thus changing the mass transfer characteristics of the microbioreactor. After injection of the inoculated medium, the needle holes created in the channels were sealed with epoxy (FIG. 42B). This was to prevent evaporation at these injection sites. Although PDMS self-seals to a large extent, we have noticed that needle holes increase the rate of evaporation and provide sites for the growth of air bubbles.

Once the microbioreactor was filled with medium it was placed inside the chamber and secured to the base. Open reservoirs of water were placed inside the chamber to provide humidity. Keeping the atmosphere within the chamber at high humidity minimizes evaporative losses through the PDMS membrane. The chamber was then closed and continuous readings were started. When fermentations were performed with pure oxygen in the chamber headspace, oxygen was passed through the chamber prior to the start of the readings.

The time between inoculation of fresh medium and placement of the filled microbioreactor in the chamber was 20 minutes. During this time the medium was kept at room temperature to minimize cell growth. The time between placement of the bioreactor in the chamber and the first reading was 10 minutes. During this time the bioreactor and cells warmed up to 37° C.

Cell Counts

Estimates of cell number from the microbioreactor and the bench-scale bioreactor were obtained using two methods. Direct cell counts were carried out using a Petroff-Hausser counting chamber and standard counting methodology. Viable cell counts were carried out using the technique of plating serial dilutions (Ausubel et al. 1995).

Medium Analysis

A series of experiments in defined medium was carried out to provide samples for off-line analysis of organic acids and glucose in both the bench-scale bioreactor and the microbioreactor.

During fermentations in the bench-scale bioreactors, samples of the medium were periodically removed, filtered, and frozen for later analysis.

Samples from the microbioreactors were obtained by sacrificing their entire volume. In order to obtain a sufficient volume of medium for analysis, the microbioreactors were fabricated to contain a volume of 50 μl. This allowed for volume loss during filtering and transfers, and provided sufficient filtered volume to meet the requirements of the HPLC protocol (5 μl). The medium samples were collected over several days. Each day three microbioreactors were inoculated and allowed to run in parallel while process parameters were measured. All three were then sacrificed at a predetermined time, and their contents were removed, filtered, and frozen. In this way, microbioreactor data was obtained at five time points.

An Agilent 1100 Series HPLC equipped with an organic acid analysis column (Aminex HPX-87H Ion Exclusion Column, Bio Rad) was used for off-line medium analysis. Samples were prepared by filtration through a 0.2 μm membrane (Pall Gelman Laboratory). Calibration was carried out by running standards at two concentrations for each of the organic acids assayed, and four different standards for glucose. A linear fit through the origin was obtained for all of the concentration ranges used.

Results

To allow the comparison of results obtained with the microbioreactor and the bench-scale reactor, a k_{L}a was measured in the microbioreactor and the operating conditions of the larger bioreactor were set so that its k_{L}a would be comparable. The calculation of the k_{L}a in the microbioreactor was based on a kinetic experiment (at 37° C.) in which the medium was allowed to come to equilibrium with nitrogen (0% DO) in the chamber headspace, at which time the headspace was flushed with air (100% DO) and continuous readings of the dissolved oxygen at the bottom of the microbioreactor were taken. Except for the absence of active stirring, this technique is similar to that of the dynamic “gassing-out” method that is commonly used for stirred bioreactors, during which the k_{L}a is extracted as a first-order rate constant using the equation below. The technique has previously been used to find the k_{L}a of a stagnant system (Randers-Eichhom et al. 1996).

$$\frac{ⅆ C}{ⅆ t} = {k}_{L} a ( {C}^{*} - C )$$

The first-order approximation of the above equation is applicable if mass transfer is slow relative to the response time of the sensor. If the time response of the sensor is potentially significant relative to that of the entire system, a second order fit can be used as in the following equation, where τ₁ is the time constant of the sensor and τ₂ is the time constant of mass transfer.

$$C ( t ) = 100 ( 1 - \frac{{τ}_{1} {ⅇ}^{\frac{- t}{{τ}_{1}}} - {τ}_{2} {ⅇ}^{\frac{- t}{{τ}_{2}}}}{{τ}_{1} - {τ}_{2}} )$$

Experimentally we found the time constant of our sensor to be ˜5 s. When response curves of our system were fit to the above equation we calculated an average k_{L}a of ˜60 h⁻¹. This is within the range of values measured in shake flasks (Maier and Buchs 2001; Gupta and Rao 2003; Wittmann et al. 2003) and shaken microtiter plates (Hermann et al. 2003; John et al. 2003b).

Experiments in defined medium were carried out in both the microbioreactors and the bench-scale bioreactors. MES buffer was added to provide some stabilization for the pH, since pH control was not implemented. The objectives were to establish the reproducibility of the microbioreactor relative to the bench-scale, and to demonstrate the feasibility of time-point sacrificing of the microbioreactors in order to carry out off-line analysis of the bioreactor medium throughout a fermentation. Three microbioreactors were sacrificed at each time point, and the medium was analyzed for glucose consumption and mixed-acid fermentation products using HPLC. In basic research or scale-up applications, this type of analysis would be desirable if an in situ sensor was not available for an analyte of interest.

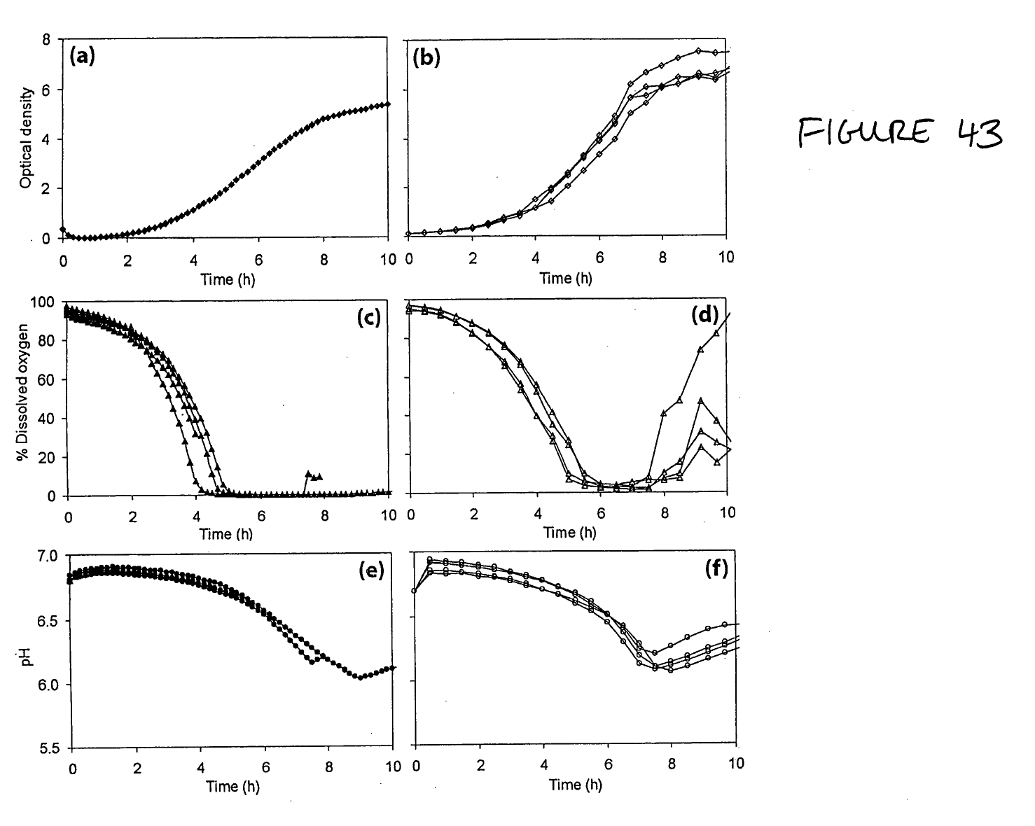

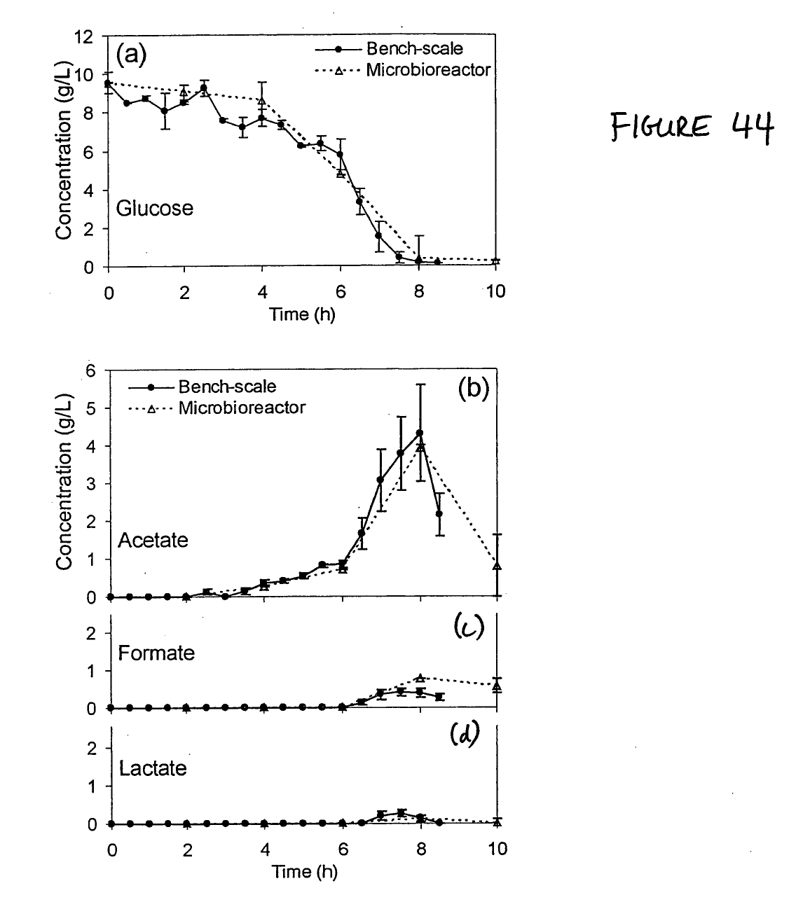

The three measured parameters within the microbioreactor and the bench-scale bioreactor are shown in FIG. 43. Each curve represents a separate run. A comparison of FIG. 43A (microbioreactors) and FIG. 43B (bench-scale bioreactors) shows that the optical density in both bioreactor types displays a similar trend, and results in a similar final OD of ˜6.

FIG. 43C and FIG. 43D show the dissolved oxygen as a function of time in the microbioreactor and the bench-scale bioreactor, respectively. Again, it can be seen that the trend in both bioreactors is similar—even though the Sixfors chambers are mixed. This result is consistent with the similar values of oxygen mass transfer (k_{L}a) for the two systems. Oxygen levels deplete during the exponential growth of cultures and eventually recover as the bacteria reach stationary phase. Because of the presence of an oxygen gradient within the vessel (as determined experimentally and from modeling), the height of the dissolved oxygen sensor foil can affect the accuracy of the measurements obtained. If the sensor is raised above the height of the microbioreactor bottom or is somehow at an angle, it will take longer to be reached by the zero-dissolved-oxygen zone during depletion, and will register dissolved oxygen earlier during reoxygenation of the medium. Depending on its height, it may never show oxygen depletion. Thus it is desirable to position the oxygen sensor such that its entire surface is exposed to the same oxygen concentration. In this case the gradient is perpendicular to the bottom of the fermentor, and the foil must then be positioned horizontally (i.e. along the bottom of the chamber), rather than on the side where readings could be ambiguous.

The variation in the microbioreactor runs appears slightly larger than in the bench-scale bioreactor runs. We believe this is most likely due to the sensitivity of the oxygen measurements in the microbioreactor to the positioning of the dissolved oxygen foil. Specifically, if any or all of the DO foil is raised above the floor of the microbioreactor, the time to depletion and the time at depletion will change due to the oxygen gradient that exists within the medium.

The trends for pH variations over time within both bioreactor types are again very similar (FIGS. 43E and 43F). It appears that this measurement exhibits less variation between runs in the microbioreactor than the DO measurement. This is most likely due to the insensitivity of the pH measurement to the positioning of the pH sensor, suggesting that a pH gradient does not exist within the microbioreactor and the bioreactor can be considered well-mixed with respect to protons.

This was confirmed experimentally by placing the pH sensor at the top of the chamber during a fermentation run. The pH curve showed the same time profile as those from fermentations in which the sensor was at the bottom. This result is consistent with the analysis of the reaction and diffusion times within the microbioreactor.

When bacteria were viewed at the end of fermentation runs, the morphology of all cultures looked normal, with no stress-induced elongation visible. Final direct cell counts in both bioreactor types were carried out, and the concentration of cells in each was found to be on the order of 10⁹ cells/ml. It is difficult to get an exact count using this method, since the depth of field on the microscope is less than the 0.02 mm depth of the counting chamber, and the small size of the bacteria results in individual cells coming in and out of focus as the focus is adjusted. However, the estimate is consistent with the numbers obtained from viable cell counts, which yielded counts of 1-4×10⁹ CFU/ml in both sizes of bioreactor.

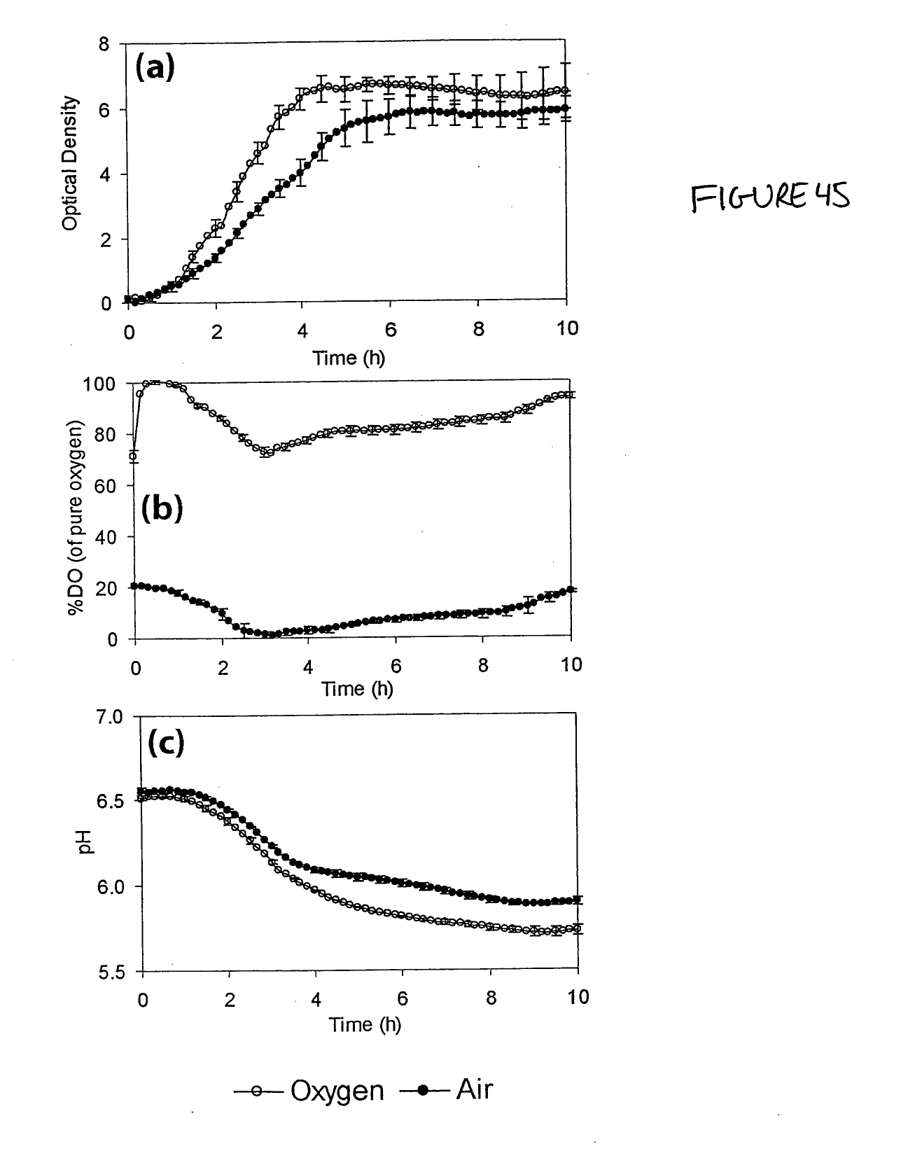

FIG. 44 shows concentration curves for the analytes measured using HPLC. The glucose uptake in the microbioreactor (FIG. 44A) corresponds closely with that in the larger bioreactor. Additionally, FIGS. 44B-44D shows that concentrations of the E. coli mixed-acid fermentation products acetate, formate, and lactate show similar trends in both bioreactor systems (succinate was not found in either bioreactor type). Acetate in particular is produced in significant amounts as the fermentation proceeds.

Fermentations with Pure Oxygen

Additional experiments were carried out in LB medium, with air as well as 100% oxygen in the headspace of the chamber (above the aeration membrane) to determine whether a difference could be observed in bacterial growth characteristics. Supplying a partial pressure of 1 atm of oxygen above the microbioreactor instead of the 0.21 atm found in air leads to an approximately five-fold increase in the solubility of oxygen in the medium, as defined by Henry's law. This approach is commonly used in large-scale fermentations to avoid oxygen limitations. An extensive literature exists on the effects of total and partial oxygen pressure on microorganisms, including E. coli. (Brunker and Brown 1971; Gottlieb 1971; Konz et al. 1998). The general consensus appears to be that partial pressures of oxygen higher than those found in air are toxic to microorganisms and inhibit their growth, but that this effect is less pronounced in a robust organism such as E. coli. Growth inhibition has been noted in E. coli in the presence of pure oxygen when minimal medium is used. It is thought that the absence of CO₂ contributes to this inhibition (Onken and Liefke 1989). Although it is known that CO₂ can inhibit microbial growth, some CO₂ may be needed by a culture growing in minimal medium for the biosynthesis of essential compounds. In a complex medium these compounds may already be present. Alternatively, fermentation of substrates within the complex medium may provide sufficient CO₂ to meet the needs of the cells. In either case, the lack of CO₂ is not inhibitory. As a result, E. coli grown in complex medium under pure oxygen conditions does not seem to show inhibited growth. The focus of the present microbioreactor study was the effect of increased oxygen levels on E. Coli growth.

In the presence of pure oxygen the initial maximum growth rate (FIG. 45A) does not appear to be different than the growth rate in the presence of air, but the bacteria are able to maintain it for a longer period of time. This is supported by the calculated doubling time in each case. With air in the headspace t_{d}=28 min±3 min, and with oxygen in the headspace t_{d}=24 min±6 min. The overlapping error bars indicate that the difference in the mean is not statistically significant (at one standard error). The maximum optical density (and thus cell count) is somewhat higher when pure oxygen is used compared to air. As stationary phase progresses, however, the optical density of cells under pure oxygen decreases until the curve coincides with the air curve. This effect could possibly be attributed to higher rates of cell lysis under pure oxygen conditions.

When pure oxygen is contacted with the aeration membrane (FIG. 45B), the oxygen within the medium shows a minimum but never depletes entirely. The minimum oxygen level that the bacteria encounter is approximately 70%. This oxygen level is still three times higher than the maximum oxygen level with air as the contacting gas. In the case of the pH time course within the microbioreactor (FIG. 45C) the error bars, representing standard error, do not show overlap at any time point beyond the beginning of the fermentation. The curves show that the pH experiences a sharper drop in the presence of oxygen than in the presence of air. This is consistent with the higher growth observed in the OD curve in the presence of pure oxygen. Since the major source of protons in the medium comes from the protons that are excluded as ammonia (existing as NH₄⁺ in the medium) crosses the cell membrane and is internalized as NH₃ (Bauer and Shiloach 1974), more growth would be expected to lead to a higher rate of proton generation, and subsequently a lower pH. At the end of fermentation runs with oxygen, bacteria exhibit normal morphology.

The results described above from the microbioreactor are reproducible in both complex medium (LB) and defined medium, and we are able to understand the oxygen transfer characteristics of the microbioreactor and effectively model growth and oxygen consumption of the bacteria during a fermentation. We have also shown that it is possible to sequentially sacrifice microbioreactors that are running in parallel to carry out off-line analysis using traditional techniques. Finally, we have shown that results obtained from the microbioreactor correspond closely with results obtained in bench-scale volumes.

### Example 9

### Design, Construction, and Operation of a Fed-Batch Microbioreactor with Active Stirring

A microbioreactor that can be used for fed-batch fermentation was constructed from polymethylmethacrylate (PMMA) and PDMS. FIG. 25A shows an expanded view of the layer structure of the microbioreactor. FIG. 25B shows a longitudinal section of the microreactor with channels and integrated magnetic stirbar (described above). The stirbar is made of neodymium-iron. FIGS. 26A and 26B show photographs of the structure. The microbioreactor includes a round cross-sectioned (i.e., cylindrical) vessel (diameter 10 mm, depth 1 mm) and three connecting channels (depth 500 microns, width 500 microns) which are used for inoculation and reagent feeding. The vessel is formed by machining a well in a PMMA body layer. A thin layer of spin-coated PDMS covers the vessel and serves as an aeration membrane to supply oxygen to the vessel interior. This thin PDMS layer is held by a thicker PDMS layer to facilitate device assembly, sealing, and microfluidic connections. Another layer of PMMA forms the uppermost portion of the structure. Voids in the thicker PDMS and upper PMMA layers allow exposure of the PDMS membrane to the external environment. Two recesses (diameter 2 mm, depth 250 microns) at the bottom of the bioreactor chamber accommodate pH and DO fluorescence lifetime sensors. A 6 mm long magnetic stir bar in the vessel center mixes the fermentation medium. The stirbar rotates around a vertical post machined out of the bulk PMMA.

Fermentations were carried out in an incubator chamber kept at 37 degrees C. by flowing heated water through its base. One inlet channel connects the microbioreactor to an elevated water reservoir. FIG. 25C illustrates the principle of passive delivery of a liquid to the microreactor vessel. The pressure passively pumps liquid at the same rate as water evaporates through the thin PDMS layer, thus keeping the volume of the microbioreactor constant. The pumping rate can be adjusted by controlling the humidity in the incubator. The cell culture was operated as a batch process when water was fed into the microbioreactor, or as a fed-batch process when other solutions (e.g. glucose or base) were drawn into the microbioreactor by water evaporation (˜μl/hr).

The incubator chamber was placed directly above a magnetic stirrer to minimize the distance to the spin bar in the microbioreactor (FIG. 31). In this set-up bifurcated optical fibers lead into the chamber from both the top and the bottom and are each connected to different LEDs and photodetectors. A transmission measurement using an orange LED (Epitex L600-10V, 600 nm) returns the optical density. The DO and pH sensor patches are excited with a blue-green LED (Nichia NSPE590S, 505 nm) and a blue LED (Nichia NSPB500S, 465 nm) respectively. Exciter bandpass filter (Omega Optical XF1016 and XF1014) and emission longpass filters (Omega Optical XF 3016 and XF 3018) separate the respective excitation and emission signals and minimize cross-excitation. Data switches multiplex the output signal and the input signal of the function generator and the lock-in amplifier, respectively, as shown in FIG. 31. All instruments are PC-controlled under a LabView software routine, which allows for automated and on-line measurement of the parameters. For the results described herein, the three parameters were read every 10 minutes.

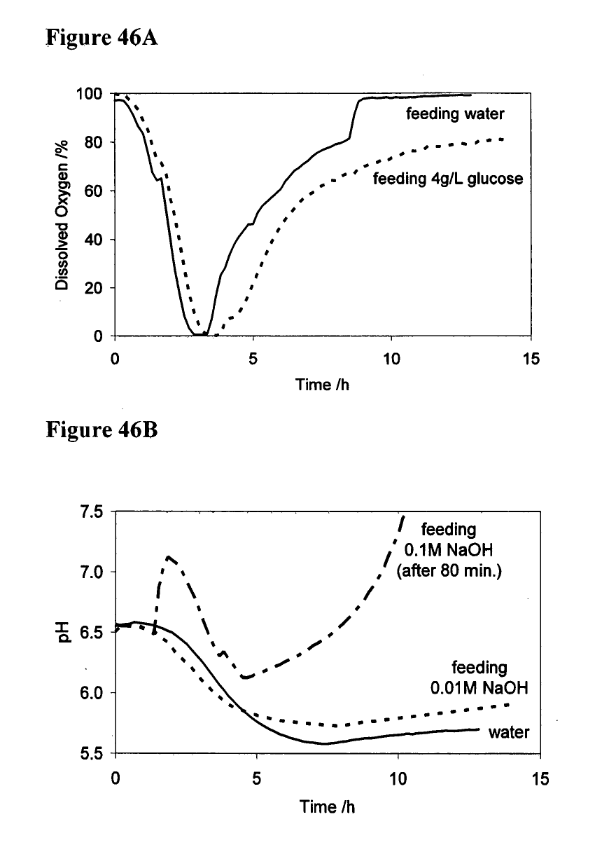

Microreactors containing optical sensors for DO and pH were inoculated with E. coli FB21591 in LB medium containing 8 g/L glucose, 100 ug/ml kanamycin, and 0.1 mol/L MES at an OD₆₀₀ of 0.05-0.07. Bioprocess parameters were monitored over time. FIGS. 46A and 46B shows results comparing operation of batch and fed-batch fermentation runs in a microreactor capable of operating in fed-batch mode. FIG. 46A is a graph showing dissolved oxygen concentration over time in a fed-batch fermentation in which the culture (E. coli) was supplied with 4 g/L glucose (dashed line) and in a batch fermentation in which the culture was supplied only with water (solid line). FIG. 46B is a graph showing pH over time in two fed-batch fermentations in which the cultures (E. coli) were supplied with 0.1 M NaOH (dot-dash line) or 0.01 M NaOH (dashed line) and in a batch fermentation in which the culture was supplied only with water (solid line).

For batch fermentation, the DO level drops rapidly to zero during exponential growth phase, when the multiplying cells have a strong demand for oxygen. As the cells enter the stationary phase, the oxygen demand drops and diffusion across the PDMS membrane returns the DO level to saturation. Addition of nutrient (glucose) appears to increase the length of the growth phase, slowing the return of the DO to saturation level.

The pH curves show a decrease to pH 5.6 in batch fermentation, which is reduced when a diluted base solution (0.01M NaOH) is fed. When a strong base solution (0.1M NaOH) is fed, pH decreases even less during cell growth phase and strongly increases thereafter. In the shown example, the strong base solution was administered 80 minutes after the fermentation run had started with cell growth in early phase.

These results demonstrate that the environmental conditions in microbioreactors can be monitored as in a batch process and manipulated in a fed-batch process. Moreover, if pH values are maintained close to neutral, cell density is expected to increase. Thus, the ability to feed base and nutrients makes this micro fed-batch system further confirms the utility of the method for screening applications in bioprocess engineering.

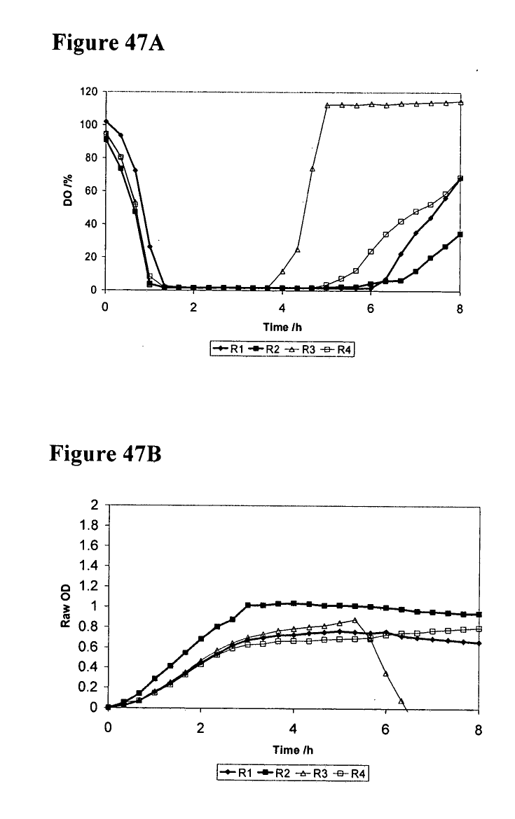

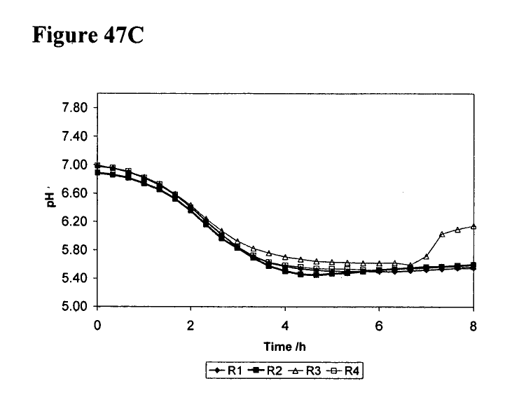

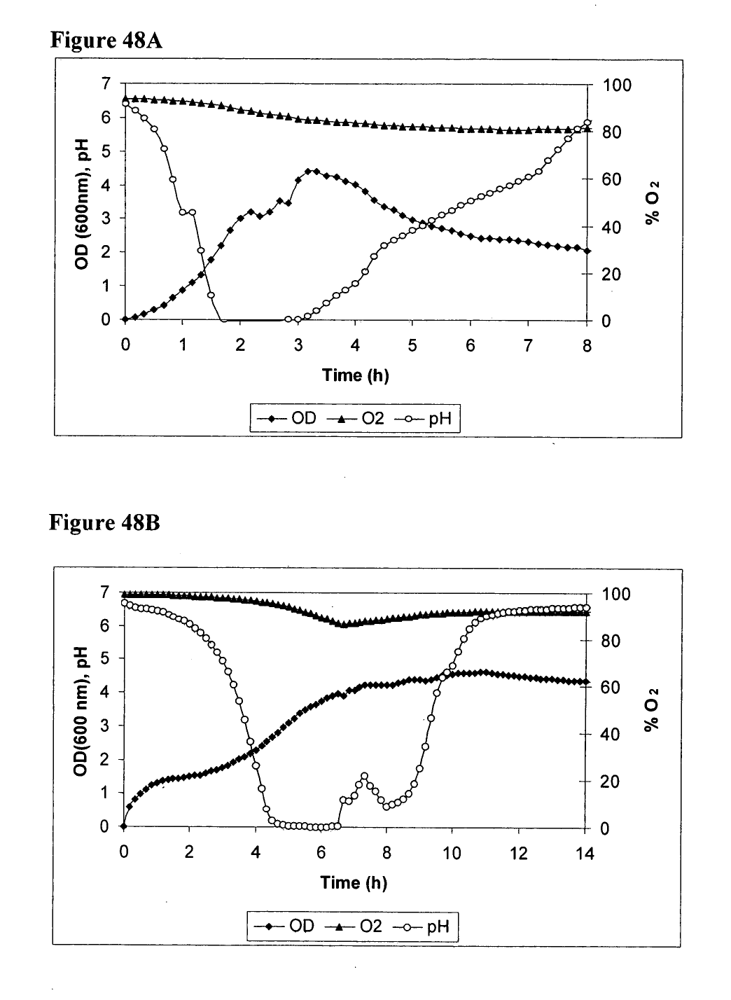

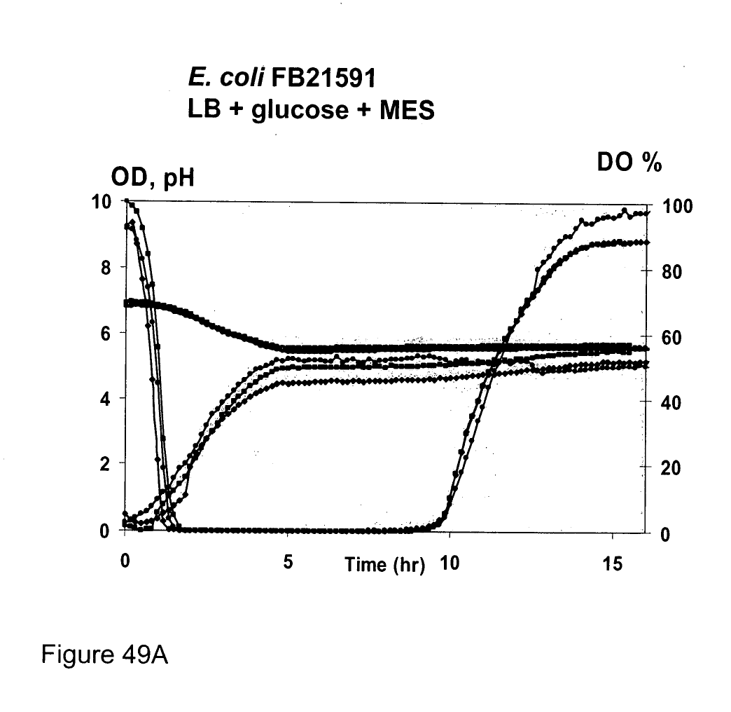

FIGS. 47A-47C, 48A-48B, and 49A-49B show additional results obtained using the microbioreactors operating under various conditions. FIGS. 49A and 49B show graphs of dissolved oxygen (DO), optical density (OD), and pH for three microreactor fermentations operating in batch mode, illustrating the high degree of reproducibility of results obtained from the reactors. FIG. 49A shows E. coli FB21591 cultured in LB+glucose+MES. FIG. 49B shows S. cerevisiae ATCC 4126 cultured in YPE+galactose.

### Example 10

### Design, Construction, and Operation of a Microchemostat with Active Stirring

A microbioreactor usable for continuous cell culture under constant growth conditions (i.e., as a microchemostat) was fabricated from polymethylmethacrylate (PMMA) and PDMS both of which were surface-modified with a PAA-g-(PEG-r-PPG) polymer as described in Example 11. The microchemostat includes a central portion similar to that described in Example 9 and illustrated in FIGS. 25A and 25B, including integrated magnetic stirbar and sensors as described in Example 9. The microchemostat includes a cylindrical vessel (diameter 10 mm, depth 1 mm) and three connecting channels (depth 500 microns, width 500 microns) which are used for inoculation, medium inflow, and medium outflow. The vessel was formed by machining a well in a PMMA body layer. The channels were created by machining depressions of the appropriate dimensions into the top and/or bottom surface(s) of the PMMA body layer so as to create three sides of a four-sided channel. The fourth side was contributed by a substrate layer beneath the body layer or by an overlying PDMS layer. Connections between channels extending inwards from the top and bottom surfaces of the body layer were created by machining perpendicular connecting channels. A thin layer of spin-coated PDMS covers the vessel and serves as an aeration membrane to supply oxygen to the vessel interior. This thin PDMS layer is held by a thicker PDMS layer to facilitate device assembly, sealing, and microfluidic connections. The thin PDMS membrane was created by spin-coating onto a silanized Si wafer and was then bonded to a thicker PDMS layer. The resulting structure was baked in an oven at 70° C. for 2 hours. The PDMS was then peeled off the wafer, and the surface was modified. The device was assembled by building a “sandwich” with the PMMA layer (already modified) containing the culture vessel below and the upper PMMA layer. The Voids in the thicker PDMS and upper PMMA layers located over the culture vessel allow exposure of the PDMS membrane to the external environment in the region overlying the culture vessel. Two recesses (diameter 2 mm, depth 250 microns) at the bottom of the bioreactor chamber accommodate pH and DO fluorescence lifetime sensors. A 6 mm long magnetic stir bar in the vessel center mixes the fermentation medium. The stirbar rotates around a vertical post machined out of the bulk PMMA.

The layer of material (body layer) containing the culture vessel extends beyond the PDMS and upper PMMA layers, as do the channels in this layer. Thus the microbioreactor device includes a central portion containing the culture vessel and extending sections on either side. One section contains a zone that is heated using a combined heater/cooler (part no. TE-7-1.0-1.3, TE technology, Inc. Traverse City, Mich., with associated controller (part number TC-24-10, TE technology). The medium inflow channel traverses the heated zone before entering the central section. This zone, which was typically maintained at approximately 70° C. during culture, serves to inhibit bacterial chemotaxis and to kill any cells that might nevertheless migrate from the culture vessel through the medium inflow channel into the heated zone.

The other extending section contains a zone that is cooled, which contains a collection chamber connected to the interior of the culture vessel via an outflow channel. Channels for sample collection and medium outflow from the collection chamber were machined into the extending section that contained the cooled zone. The cooled zone, which is typically maintained at approximately 4° C., serves to reduce cell metabolism so that cells within the collection vessel remain in essentially the same physiological state between the time they leave the culture vessel and the time the sample is removed from the collection chamber.

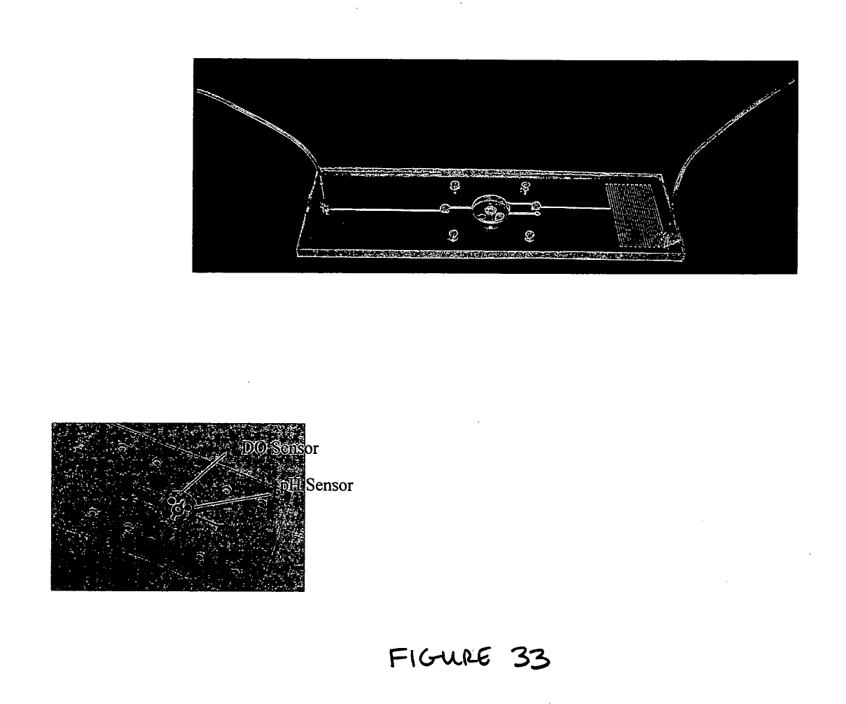

In order to minimize the effects of the heated and cooled zones on the temperature in the culture vessel, the extending sections are each connected to the central section of the body layer by three narrow “bridges”, which separate the central section of the material layer that contains the culture vessel from the bulk regions of the sections extending on either side. The medium inflow and outflow channels each extend through one of the bridges on either side of the central section. The thickness of the PMMA layer is ⅛ in (3.2 mm), and the 3 bridges are located at the both ends and the center so as to connect the edges of the central and side sections. The length and width of the bridges are 3 mm, resulting in a total contact area between the two sections of (3 mm) (3.2 mm) (3). Modeling of the temperature gradients indicated that the effects of heating and cooling on the temperature in the culture vessel were negligible. FIG. 32B provides a pictorial representation of the model results, in which color corresponds to temperature. The modeling was performed using FEMLAB® Chemical Engineering Modules (3-D simulation, heat transfer model). The dimensions of the device are 1.3 in (33 mm) by 5.6 in (142 mm) by 3.2 mm. The heat transfer coeffient for PMMA=0.18 W/(m·K), density=1190 kg/m3, and heat capacity=1450 J/(kg·K). These values were taken from the average of the range for the properties. FIG. 33 shows photographs of the microchemostat system.

Fermentations were carried out in an incubator chamber kept at 37° C. by flowing heated water through its base. One inlet channel connects the culture vessel to an elevated water reservoir. An outlet channel connects the culture vessel to a collection chamber, which is connected to an effluent receptacle. For continuous culture, as here, the microbioreactor is fed with fresh medium by pressure driven flow, either using a syringe pump or from an elevated medium reservoir. The other side of the reactor is connected with a water reservoir that serves as an effluent collector and maintains a constant volume of medium in the culture vessel (150 μm). The syringe pump can either be used to exert positive pressure on the medium reservoir (e.g., within the syringe) or to exert negative pressure on the effluent collector. In either case, fresh medium is driven into the culture vessel via the medium inflow channel and withdrawn from the vessel via the medium outflow channel. The maximum evaporation rate from the culture vessel into dry air was measured to be 4 uL/hr under natural convection conditions and is normally much less than this value. It is therefore negligible compared to the medium flow rate and could be further reduced by humidifying the incubator chamber.

The incubator chamber was placed directly above a magnetic stirrer to minimize the distance to the spin bar in the microbioreactor, as described in Example 9. The setup for optical excitation, signal collection, data processing, and control was as described in Example 9. Microbioreactors containing optical sensors for DO and pH were inoculated with E. coli FB21591 in LB medium containing 8 g/L glucose, 100 μg/ml kanamycin, and 0.1 mol/L MES at an OD₆₀₀ of 0.05-0.07. Bioprocess parameters were monitored over time in a series of experiments with medium inflow rates of between 0.8 μl/min and 2 μl/min (i.e., dilution rates of between 18.75 hr⁻¹ and 75 hr⁻¹), which were controlled by appropriately setting the syringe pump.

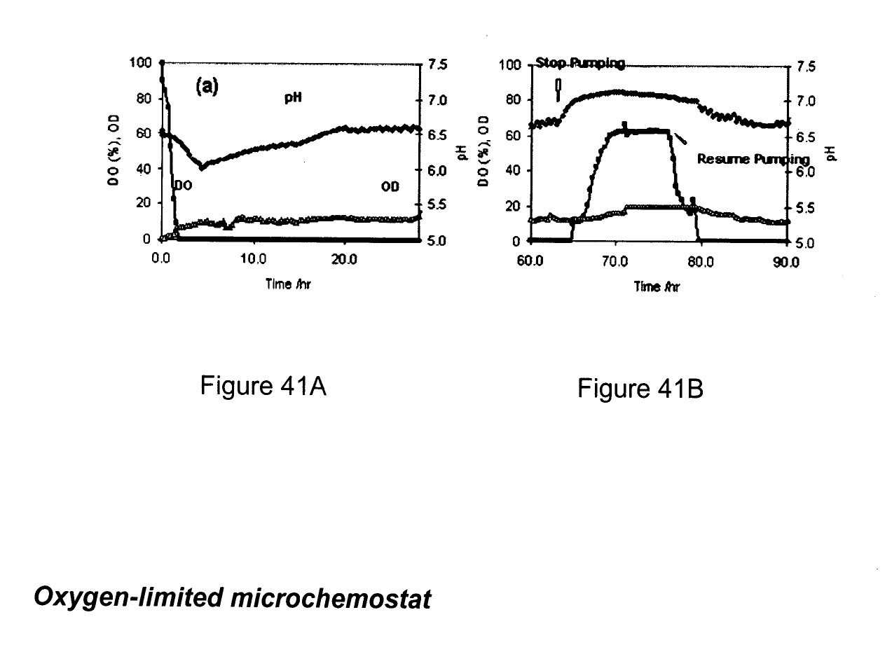

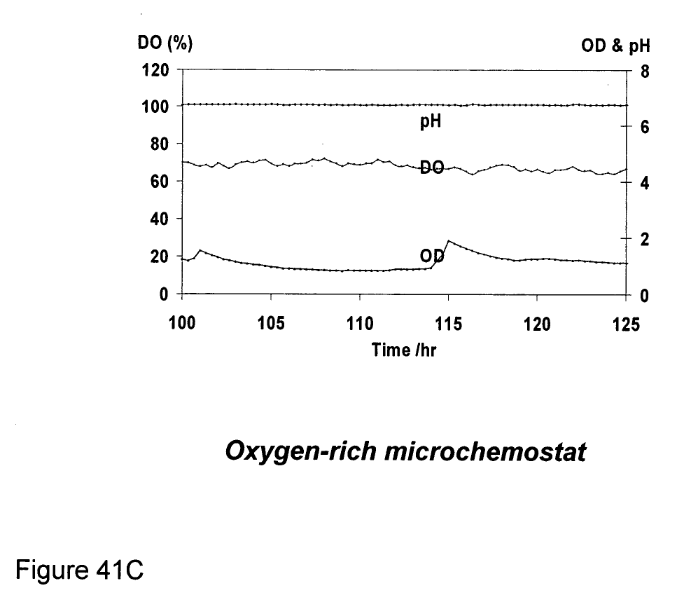

FIGS. 41A and 41B show results of a representative experiment in which a syringe pump exerting positive pressure was used to drive medium through the culture vessel chamber at 2 μl/min. FIG. 41A is a graph showing dissolved oxygen concentration (DO; solid line), pH (diamonds), and optical density (OD; triangles), reflecting biomass) over time. These parameters change rapidly during the early stages of culture, as the cells utilize all available oxygen and biomass increases. As a result, the DO level drops to 0 in about 2 hours. The pH level of the culture medium decreases during the initial phases of culture as a result of cell metabolism and then increases with the supply of fresh medium as the rate of biomass increase begins to slow down. Under these conditions the culture is oxygen limited. At about 20 hours DO, pH, and OD reach stable levels, and chemostat conditions are established. The culture is predicted to remain in steady state indefinitely.

FIG. 41B is a graph showing dissolved oxygen concentration (DO; solid line), pH (diamonds), and optical density (OD; triangles), reflecting biomass) over time in the same culture, starting at a later point in time. The values of these parameters at 60 hours are identical to those at earlier time points following achievement of steady state (e.g., 20 hour time point in FIG. 41A, indicating that steady state has been maintained. Pumping was temporarily stopped at approximately 63 hours, depriving the cells of an ongoing source of nutrients. The DO concentration and pH rose rapidly as the culture became nutrient limited. Since the medium flow rate was 0, chemostat conditions were temporarily interrupted. Pumping was resumed at about 78 hours (15 hours after pumping was turned off). Following the resumption of pumping, the DO and pH declined, as the culture became oxygen-limited again. The experiment was terminated before a new steady state was reached due to exhaustion of the medium reservoir, but it is evident that a return to steady state was occurring.

FIG. 41C shows operation of the microchemostat under oxygen rich conditions, in which nutrient concentration was limiting. The microbioreactor was inoculated with E. coli HCB137 strain (gift from Prof. H. Berg, Harvard University). The medium composition was 5 g/L tryptone+1 g/L glucose+5 g/NaCl, 0.1 mol/MES, Tet antibiotic. The flow rate was 0.8 μl/min. The temporary increase in OD at 101 and 115 hours is due to the fluctuation of pressure inside of the reactor. Since the PDMS membrane is very thin, a small pressure difference can cause the PDMS to bulge slightly, resulting in noise in the OD reading due to temporary change in volume. However, the culture condition in the chamber remain undisturbed since the DO level remains stable during this period. Since DO is very sensitive to changes in medium flow rate, this stability indicates that the flow rate remained constant.

The results described above demonstrate that the microbioreactor can be operated as a microchemostat in conjunction with appropriate pumping system, medium reservoir, effluent collector, etc., and can maintain constant culture conditions of nutrient concentration, dissolved oxygen concentration, and pH, over a prolonged period of time, resulting in a constant rate of biomass production (i.e., cell density remains constant). Changes in parameters such as the medium inflow rate will result in a shift to a new steady state. This experiment demonstrates operation under oxygen limited and nutrient limited conditions. Together with the ability to continuously monitor dissolved oxygen, pH, and biomass and to sample the medium and cells leaving the culture chamber, this system provides a powerful and flexible tool for the analysis of cell physiology and biochemistry, metabolic flux, gene expression, product formation, etc., under a wide variety of conditions with minimal use of reagents and production of waste, allows the optimization of culture parameters and strains, and can be used to provide critical information for the engineering of improved metabolic pathways.

### Example 11

### Modification of Microbioreactor Surfaces

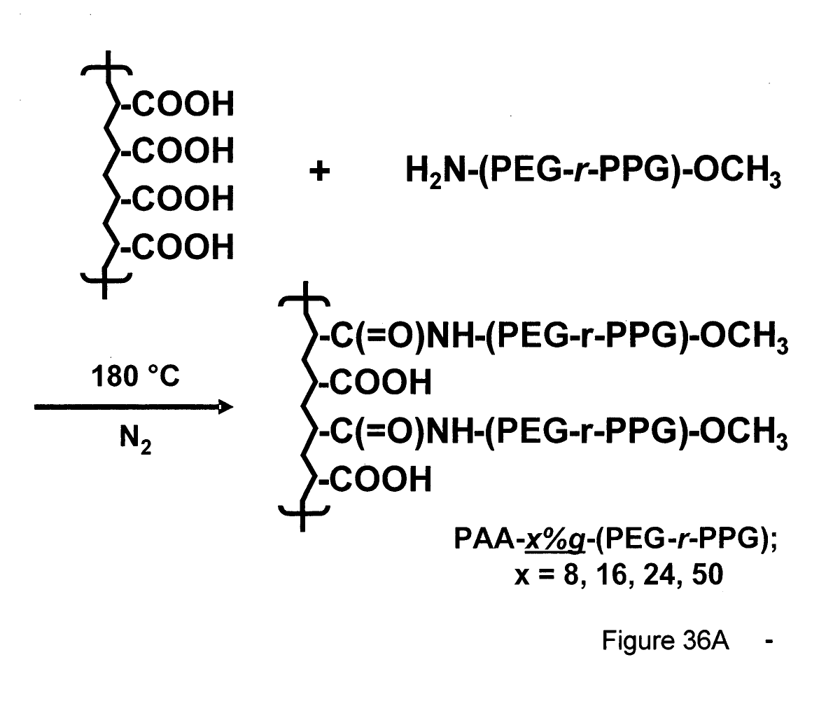

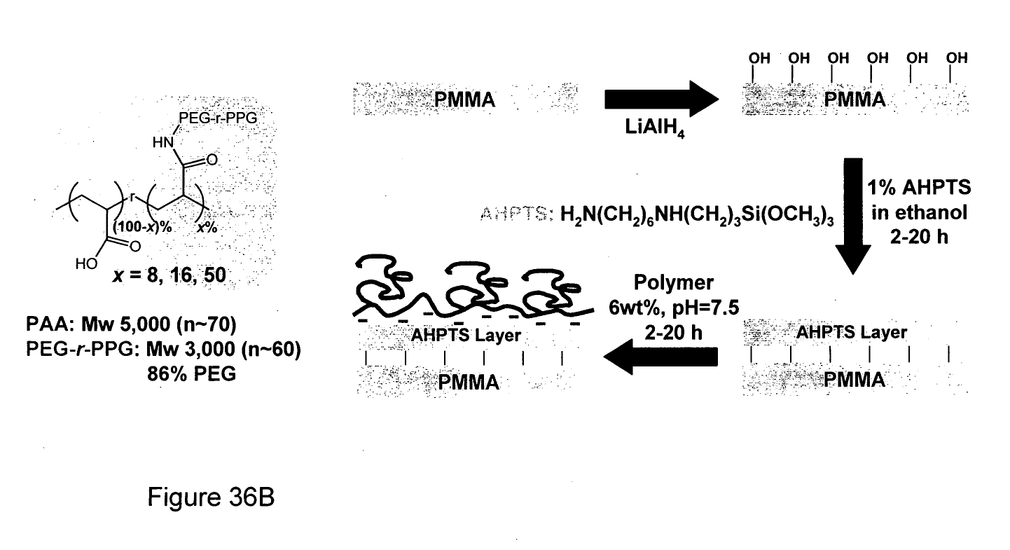

To reduce the adherence of cells and/or proteins to the interior surfaces of microbioreactor culture vessels, channels, and sensors, techniques were developed to coat such surfaces with poly(ethylene glycol) (PEG)-containing polymers. Polymers composed of amine-terminated linear poly(ethylene glycol-r-propylene glycol) (PEG-r-PPG) (86%:14%) (Jeffamine® XJT234, PEG-r-PPG-NH₂, Huntsman Corp—catalog #:XJT234, PEG:PPG=86:14, MW: 3000) were grafted onto PAA (Aldrich—catalog #:19203-1, 50 wt % solution in water, MW: 5000) at grafting densities of 8%, 16%, 24%, and 50% as shown schematically in FIG. 36A to generate PAA-g-(PEG-r-PPG) comb polymers (128, 130).

The scheme for PMMA modification is shown in FIG. 36B. PMMA surfaces were first reduced by treatment with LiAlH₄, which reduces PMMA ester groups (—O—C═O) to OH. Other reducing agents such as sodium borohydride could also have been used. A series of experiments was performed to determine the optimal conditions for maximizing OH group production. Optimum conditions were found to be 30 min, 0.4 M LiAlH₄ in ether solution at room temperature. For a polymeric material such as PMMA, the selection of an appropriate solvent is extremely important since many organic solvents dissolve the polymer substrate and cause deformation. Here, diethyl ether was selected in order to avoid such problems with PMMA. The reduced PMMA was then soaked in an ethanolic solution of N-(6-aminohyexyl)-aminopropyl trimethoxysilane (AHPTS) (Gelest, Inc.—catalog #: SIA0594.0, 95% for 2-20 hours (typically 20 hours) (1% AHPTS in ethanol) to form an amine-terminated self-assembled monolayer (SAM) coating (129). The PMMA/AHPTS surfaces were then soaked in an aqueous solution of a PAA-g-(PEG-r-PPG) polymer (polymer was at 6% by weight, pH=7.5) for 2-20 hours (typically 20 hours) to form a polymer film in which the PAA-g-(PEG-r-PPG) polymer was adsorbed onto the PMMA surface by electrostatic interactions with the exposed amine-terminated SAMs. Further characterization of the modified surfaces could be done as described below for PDMS.

To test the resistance of modified PMMA towards cell adhesion, unmodified PMMA, PMMA with surfaces modified with PAA-g-(PEG-r-PPG) polymer, or PMMA modified with a layer-by-layer (LBL) assembled poly(acrylic acid) and poly(acrylamide) (PAAm) multilayer (10 bilayers) was put into petri dishes, autoclaved, inoculated with either E. coli (FB21591), S. cerevisiae (ATCC 4126), or fibroblasts (ATCC CCL110), and cultured in the appropriate medium for varying periods of time. E. coli (FB21591) were inoculated in a modified Luria-Bertani (LB) medium containing 10 g/L of trypton (Difco Laboratories), 5 g/L of yeast extract (Difco Laboratories), and 5 g/L of NaCl, 8 g/L glucose, 19.52 g/L MES (100 mM final concentration) and 100 μg/mL kanamycin, at an OD₆₀₀=0.056, pH=6.7 and maintained in culture for 20 hr at which time the OD₆₀₀ was 2.21 and the pH was 5.6. S. cerevisiae (ATCC 4126) were inoculated in YPE medium was used, which is composed of 10 g/L of bacto yeast extract (Difco), 5 g/L of bacto peptone (Difco), 20 g/L of glucose and 50 μg/mL of streptomycin at an OD₆₀₀=0.06, pH=3.9 and maintained in culture for 20 hr at which time the OD₆₀₀ was 4.29 and the pH was 3.8. Fibroblasts (ATCC CCL110) were inoculated in Eagle's minimal essential (EME) medium with Earle's balanced salt solution (BSS) and 2 mM L-glutamine, which consisted of 1 mM sodium pyruvate, 0.1 mM nonessential amino acids, 1.5 mg/mL sodium bicarbonate, 0.01 mg/mL bovine insulin, and 10% fetal bovine serum, at 37 degree C. in humidified air containing 5% CO₂ at an initial cell density of 2×10⁴ cells/ml and maintained in culture for 5 days.

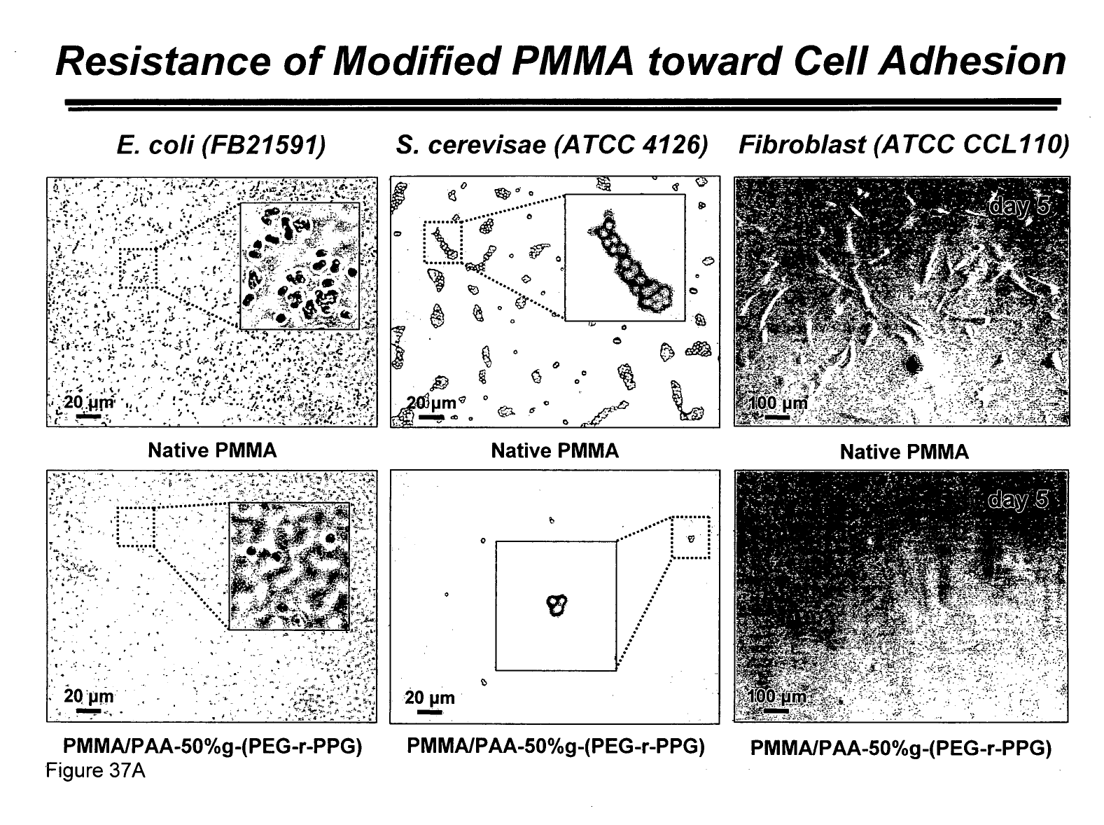

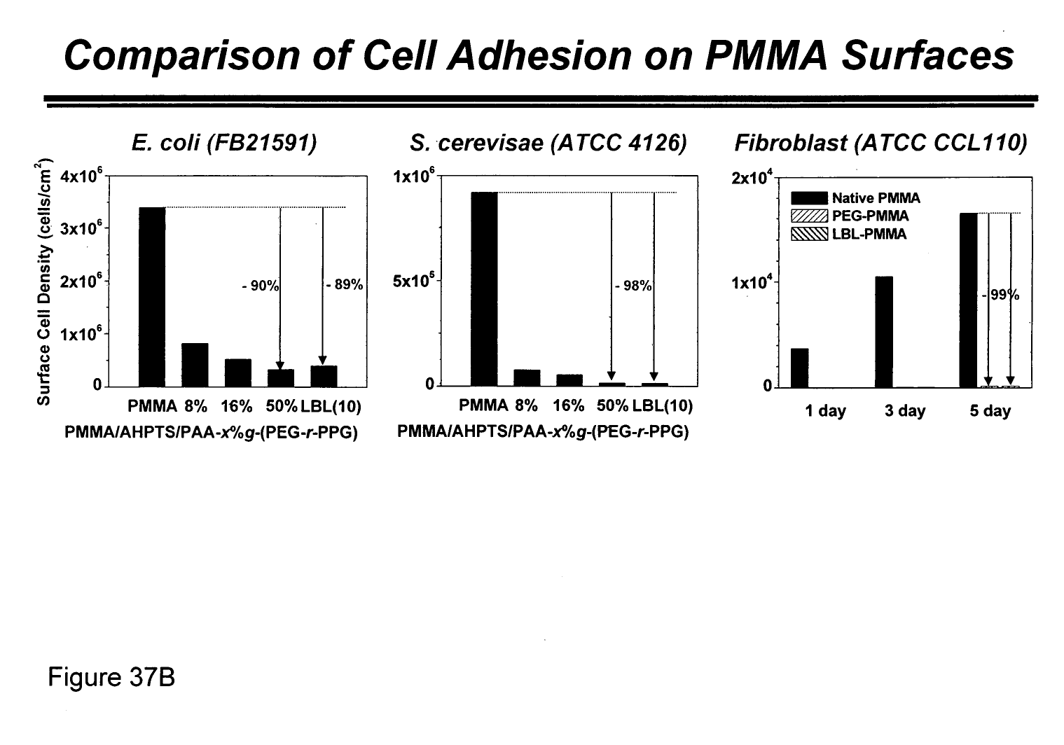

FIG. 37A shows photographs taken with an optical microscope, comparing the unmodified surfaces and surfaces modified with PAA-g-(PEG-r-PPG) polymer (50% grafting density) for each of the 3 cell types. It is evident that modification greatly reduced the density of adherent cells in all cases. FIG. 37B presents the results in quantitative form 20 hours after inoculation for E. coli and S. cerevisiae and at 1, 3, and 5 day time points for fibroblasts. Data for E. coli and S. cerevisiae show that adherence decreased with increasing graft density. At 50% grafting density, the density of adherent cells on modified PMMA was reduced by 90% (E. coli), 98% (S. cerevisiaie), or 99% (fibroblasts) relative to unmodified PMMA, i.e., approximately the same amount as achieved with LBL PAA/PAAm modification.

For PDMS modification, surfaces were first oxidized by O₂ plasma treatment for 30 s to generate free OH groups and then soaked in an ethanolic solution of N-(6-aminohyexyl)-aminopropyl trimethoxysilane (AHPTS) (2% AHPTS in ethanol) to form an amine-terminated self-assembled monolayer (SAM) coating as described for PMMA. The PDMS/AHPTS surfaces were then soaked in an aqueous solution of a PAA-g-(PEG-r-PPG) polymer to form a polymer film, also as described for PMMA.

Each step in the PDMS modification procedure was characterized by X-ray photoelectron spectroscopy (XPS). High resolution C(1s) XPS spectra showed an increase of a C—O peak (285.7-286.2 eV) upon polymer adsorption, and additional increases in the C—O peak and decreases in the C—C peak (283.6-284.0 eV) with increases in the PEG grafting ratio, indicating the successful coating of the PDMS surfaces with PAA-g-(PEG-r-PPG) polymer films. The resulting polymer coatings were stable with respect to high salt concentrations, and to sonication. The modified surfaces were hydrophilic, with contact angle θₐ(H₂O)=35±5°.

The changes in the surface properties of the PDMS upon modification was demonstrated by wetting experiments. A microchannel with a height of 60 μm and a width of 130 μm was modified by sequential flows of the AHPTS solution (20 μl/min for 3 hours) and then the PAA-g-(PEG-r-PPG) polymer solution (5 μL/min for 18 hours). After rinsing with deionized water and drying at 70° C. for 1 day, a stable hydrophilic PDMS channel was formed. FIG. 37C shows the wetting of the PDMS microchannel 5 days after the surface modification. The modified channel exhbited enhanced wettability and water could be drawn into the channel without external pressure, whereas external pressure had to be applied to push water into the unmodified PDMS channel.

The protein resistance of the PAA-g-(PEG-r-PPG)-modified PDMS was evaluated by high resolution N1(s) XPS spectra. Since an amino acid contains at least one nitrogen atom, the density of the N(1s) signal in XPS can be used as a metric for comparing the relative amounts of adsorbed proteins on different surfaces. Unmodified or PAA-g-(PEG-r-PPG) polymer-coated PDMS surfaces were soaked in PBS buffer solutions that contained 0.25 mg/ml insulin, lysozyme, hexokinase, or fibrinogen. After 20 hr of exposure, the PDMS surfaces were rinsed with deionized water and dried in a nitrogen stream. The relative amounts of adsorbed protein were estimated by the N(1s) signal in an XPS measurement. As shown in FIG. 37D, the nitrogen signals for the PAA-g-(PEG-r-PPG)-modified PDMS were reduced for all four proteins compared with native PDMS. There was a further increase in the N(1s) signal with increasing PEG-grafting ratio. These results strongly suggest that the PAA-g-(PEG-r-PPG)-modified PDMS surfaces are effective in reducing non-specific adsorption of proteins.

The resistance of PAA-g-(PEG-r-PPG) polymer films to non-specific cell adsorption was also investigated by culturing E. coli (DPD2417) on modified or unmodified PDMS surfaces as described above for PMMA. The number of cells adsorbed onto the modified PDMS surface was ˜10% of that on native PDMS (data not shown). These results indicate that surface modification with PAA-g-(PEG-r-PPG) copolymer reduces adhesion of cells onto the PDMS surface.

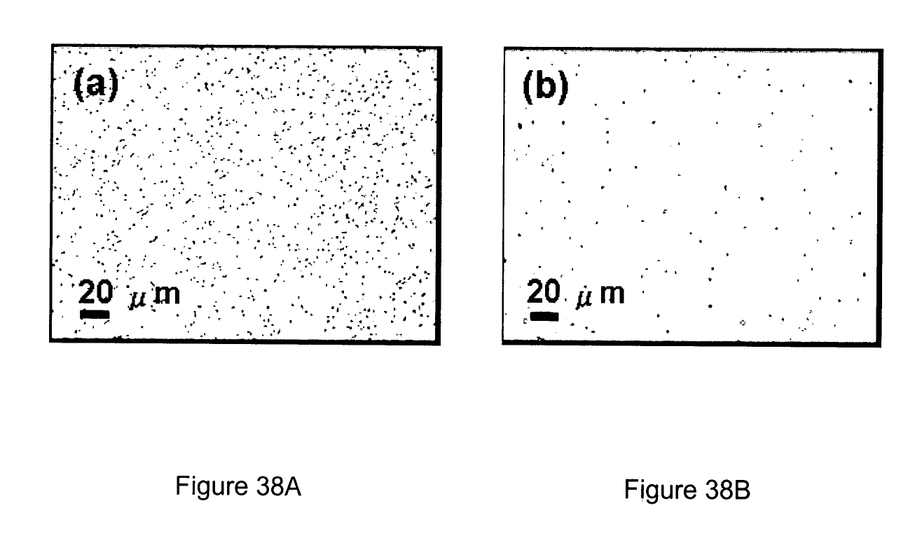

To evaluate the ability of the PAA-g-(PEG-r-PPG) modification to inhibit cell adherence in the context of a microbioreactor, E. coli were inoculated in rich medium at low density in microbioreactors such as that described in Example 9, in which PMMA and PDMS interior surfaces were either unmodified or were modified with PAA-g-(PEG-r-PPG) polymer (50% grafting density). Briefly, PDMS was spin-coated on a silanized wafer and then bonded to a thicker PDMS layer. The structure was then baked at 70° C. for 2 hours. The PDMS and PMMA layers were dipped in the polymer solution as described above and then assembled to form a complete microbioreactor device. The culture vessels were inoculated with cells and maintained with constant mixing for 24 hours. The microbioreactors were then opened and PMMA surfaces from the interior of the culture vessel were photographed using an optical microscope following staining with safranin. FIG. 38 shows results of this experiment. FIG. 38A shows an unmodified PMMA surface while FIG. 38B shows a modified PMMA surface. It is evident that modification greatly inhibited cell adherence, reducing it to 10% or less of the value obtained without modification.

### Example 12

### Construction of a Multilayer Microbioreactor and its Testing Under Continuous Culture Conditions

Microreactor Materials Design and Fabrication

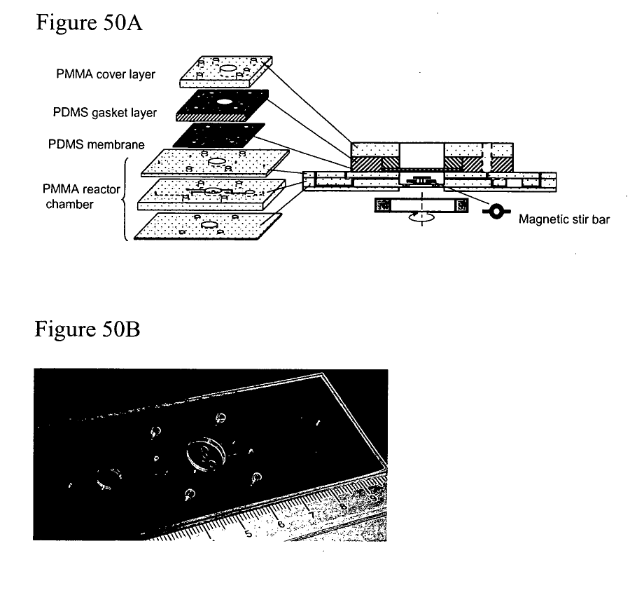

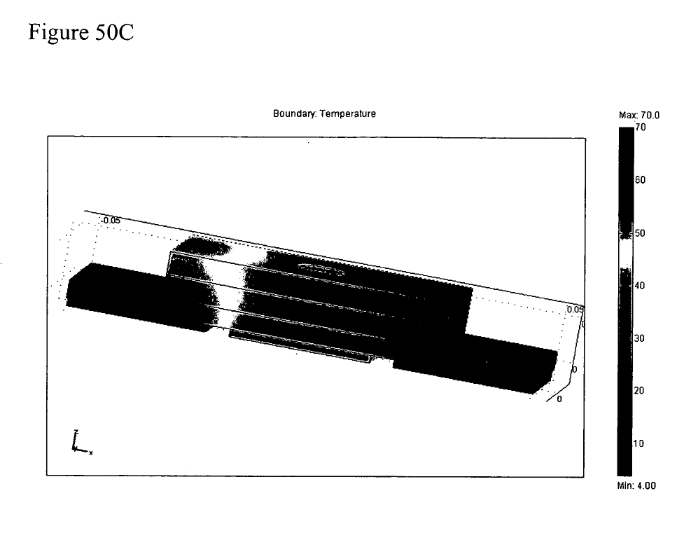

The microbioreactor consisted of four PMMA layers and two PDMS layers (see FIG. 50A). The microbioreactor chamber (diameter 10 mm, depth 2 mm, total volume of 150 μL) and three connecting channels (depth 250 μm, width 250 μm) were fabricated in three bottom PMMA layers (1 mm, 1.5 mm, and 0.5 mm in thickness, Goodfellow Corp., Devon, Pa., USA) by using a computer-numerical-controlled (CNC) milling machine and thermally bonded using a home-made mechanical press (140 kPa, 145° C. for 90 mins). A thin layer (100 μm) of spin-coated PDMS (mixing ratio of silicone to curing agent was 10:1. Sylgard 184, Dow Corning Corp., Midland, Mich., USA.) covered the reactor chamber and served as the aeration membrane. PDMS was spin-coated at a speed of 1200 rpm for 25 seconds and then baked at 70° C. for 2 hours for curing. To facilitate device assembly and hermetic sealing, this PDMS layer was held by a 5 mm-thick PDMS gasket layer. The PDMS layer was covered with an additional layer of stainless steel grid (B-PMX-062, Small Parts Inc., Miami, Fla., USA) fixed by a home-made PDMS O-ring to provide a perforated membrane structure. A top PMMA layer was used to provide a rigid support for mechanical assembly.

In the reactor chamber, two recesses (diameter 2 mm, depth 250 μm, 2.7 mm radial distance from the center) at the bottom of the bioreactor chamber accommodated pH and DO fluorescence lifetime sensors (DO sensor foil PSt3, and pH sensor solution HP2A, PreSens—Precision Sensing GmbH, Regensburg, Germany). A ring-shape magnetic stir bar with 6 mm arm length and 0.5 mm thickness (custom-made by Engineered Concepts, Vestavia Hills, Ala., USA) was used for active mixing. The rotation of the stir bar was horizontally defined by a free-standing vertical post (height of 800 μm, diameter of 1.35 mm) and vertically defined by a shallow shoulder (height of 200 μm, diameter of 2.2 mm) machined out of the bulk PMMA in the center of the reactor chamber. A piece of PMMA with 250 μm in thickness and 3 mm in diameter was attached on top of the PMMA post by using acrylic solvent (Weld-On 4, IPS Corp., Gardena, Calif., USA) to keep the magnetic stir bar in position (FIG. 50A).

Fluidic Connection and Temperature Control

Small connecting ports (660 μm in diameter) were drilled into the PMMA chip at two inlets (for inoculation and medium feeding purposes, respectively) and two outlets (for exit to waste and sampling) of the microbioreactor. Stainless steel tubes (23 gauge, Small Parts, Inc., Miami Lakes, Fla., USA) were fixed into these ports by epoxy and connected to polyethylene tubings ( 1/32″ outer diameter, Becton Dickinson, Franklin Lakes, N.J., USA). Fresh medium in a 10 mL glass syringes (Gastight, Becton Dickinson and Company) was pumped and fed to the microbioreactor by a syringe pump (PHD2000, Harvard Apparatus Inc., Holliston, Mass., USA). The other side of the reactor was connected to a pressurized water reservoir (elevated at a height of 300 mm) that served as the effluent collector and also kept the reactor at a constant, positive pressure.

Different from chemostats using conventional gas bubble-sparged, stirred-tank bioreactors, in the microchemostat medium continuously flows through the microbioreactor as a single phase flow to eliminate potential disturbances in flow rates caused by surface tension effects at small scales. As a consequence of the single phase flow, motile bacteria, e.g. E. coli, could potentially swim upstream into the medium reservoir for nutrients. In order to reduce chemotaxis, the cross-sectional dimensions of the microchannels were chosen as 250 μm×250 μm. With a typical flow rate ranging from 0.5 μL/min to 2 μL/min, corresponding average linear flow rates were of 130˜500 μm/s, which is significantly higher than the average migration speed of E. coli cells (20˜80 μm/s) (Brock et al., 1994). As a result, only a very small fraction of cells was able to swim back up the feeding channel. To further reduce the potential for cell migration, we used a local heater (HP-127-1.0-0.8P, TE Technology, Inc., Traverse City, Mich., USA) to raise the temperature of the feed line to ˜70° C. and thereby reverse the driving force for chemotaxis, since the cells have adverse chemotaxis towards high temperature (Maeda et al., 1976; Alder, 1976). The high temperature zone had the additional advantage of killing cells that may reach the heating zone. At the exit side of the microbioreactor, a peltier thermoelectrical cooler (HP-127-1.0-0.8P, TE Technology) reduced the local temperature of a 40 μL effluent reservoir/collection chamber (1.5 mm deep and 6 mm in diameter) to 4° C. to keep cells at low temperature and significantly reduce metabolic activity to facilitate off-line sampling for further analysis. A thin piece of copper (1 mm in thickness) was placed between the heater/cooler module and the microbioreactor; temperature was measured by a thermal couple (TP-2444, TE Technology) and feedback-controlled a temperature controller (TC-24-10, TE Technology). Temperature distribution in the microbioreactor was simulated by finite element method using Femlab®software (version 3.1, Comsol, Inc., Burlington, Mass., USA) and shown in FIG. 50C. The microreactor chamber and microchannels are located at the bottom side of the device, thus temperature disturbance by native convection of air is not significant. Temperature of the reactor chamber, where fermentation was performed, was carefully controlled and maintained at 37° C.

Optical Measurement Setup

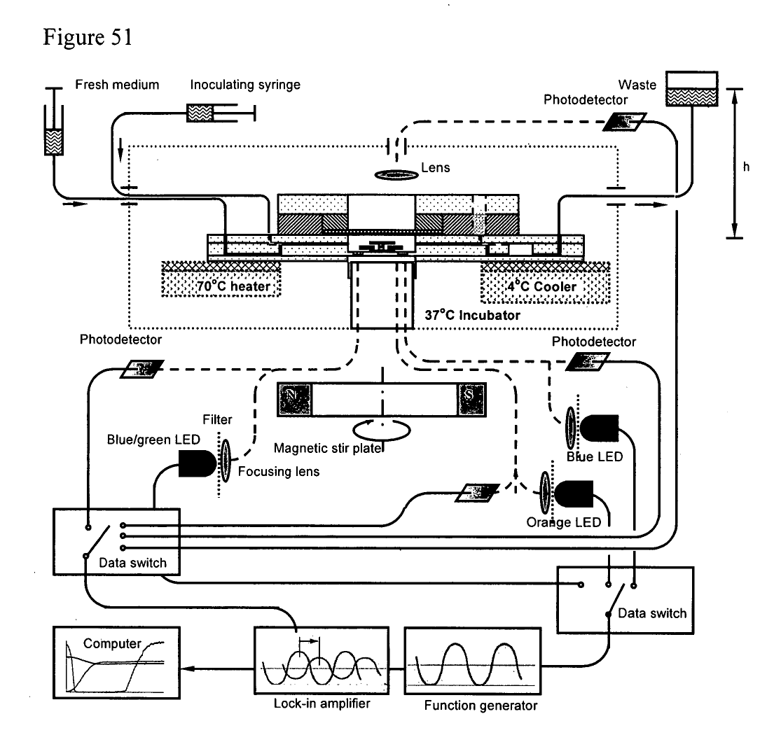

The experimental set-up is shown in FIG. 51. DO, pH, and OD were measured by the optical sensing methods described in detail elsewhere herein and in Zanzotto, et al. (2004), and only a brief summary is included here. The microbioreactor was placed in an aluminum chamber maintained at 37° C. by flowing heated water through its base. An external magnetic stirrer (SP72725, Barnstead International, Dubuque, USA) was placed directly below the aluminum chamber and drove the movement of the ring-shape stir bar in the microbioreactor. Bifurcated optical fibers (custom-made by RoMack Fiber Optics, Williamsburg, Va., USA) led into the chamber from both the top and the bottom and connected to LEDs and photodetectors (PDA-55, Thorlabs, Newton, N.J., USA) to perform the optical measurements. Both dissolved oxygen and pH were measured using phase modulation lifetime fluorimetry. The DO and pH sensors were excited with a blue-green LED (505 nm, NSPE590S, Nichia America Corporation, Mountville, Pa., USA) and a blue LED (465 nm, NSPB500S, Nichia), respectively. Excitation bandpass filters (XF1016 and XF1014, Omega Optical, Inc., Brattleboro, Vt., USA) and emission longpass filters (XF3016 and XF3018, Omega Optical) separated the respective excitation and emission signals and minimized their cross-excitation. OD data, closely related to biomass concentration in the microbioreactor, were obtained from an absorbance measurement using an orange LED (L600-10V, 600 nm, Epitex, Kyoto, JP). The bifurcated branch yielded a reference signal to compensate for intensity fluctuations of the orange LED. Data switches (7204, Electro Standard Laboratories, Cranston, R.I., USA) multiplexed the output signal and the input signal of the function generator (33220A, Agilent Technologies, Palo Alto, Calif., USA) and the lock-in amplifier (SR 830, Stanford Research Systems, Sunnyvale, Calif., USA), respectively. All instruments were PC-controlled under LabVIEW® (National Instruments Corp., Austin, Tex., USA), which enabled automated and real-time measurement of the parameters.

Surface mModification Procedure for PDMS and PMMA

A PAA-g-(PEG-r-PPG) polymer coating on both PDMS and PMMA surface are employed to reduce cell adhesion. PAA-g-(PEG-r-PPG) graft copolymer was synthesized as described in Example 11 using an amidation reaction to graft H₂N-(PEG-r-PPG)-OCH₃ (Jeffamine XTJ-234, Huntsman Co., Houston, Tex., USA) chains to the carboxylic acid groups on the PAA (Sigma-Aldrich, Co., St. Louis, Mo., USA) backbone (Moeser et al., 2002) with a grafting ratio of 50%. In our typical synthesis, a total of 23 g of the two polymers in the desired stoichiometric ratio was added to a reaction vessel. The mixture was heated to 180° C. and left to react for 2 h under a bubbling flow of N₂ that provided mixing, prevented oxidation, and expelled water produced by the condensation reaction. The product was cooled to room temperature and dissolved in de-ionized water to produce 33 wt % stock solution. Completion of the reaction was verified by the disappearance of free amine in a Ninhydrin test.

The surface modification protocols started with 30 seconds O₂ plasma treatment (0.15 Torr O₂ pressure. PDC-32G, Harrick Scientific) for PDMS and reduction with LiAlH₄ (0.4 mol/L for 30 mins of reaction time. Sigma-Aldrich) for PMMA to generate surface hydroxyl groups. Upon the reduction of PMMA surfaces, randomly aligned small chain segments were produced and subsequently the surface OH groups were formed on these chains. PDMS and PMMA layers were then immersed in a solution of 1 wt % ethanol solution of N-(6-aminohexyl)aminopropyltrimethoxysilane (AHPTS, Gelest, Inc. Morrisville, Pa., USA) for 24 hours. After being removed from solution, rinsed with ethanol and dried under the stream of N₂, AHPTS-coated PDMS and PMMA layers were assembled into a microbioreactor. Aqueous solution of PAA-g-(PEG-r-PPG) (6 wt %, pH 7.4) was pumped through the microbioreactor, followed by rinsing with distilled water and drying under N₂.

Material and Methods for Biological Experiments

E. coli FB21591 (thiC::Tn5-pKD46, Kan^{R}), a derivative of E. coli K12, was obtained from University of Wisconsin and used as a model organism. Two culture media were used for different experiments: Luria-Bertani (LB) rich medium containing 8 g/L glucose (Mallinckrodt, Hazelwood, Mo., USA), 100 mg/L kanamycin (Sigma-Aldrich), and 0.1 mol/L 2-(N-morpholino) ethanesulfonic acid (MES) (Sigma-Aldrich), and MOPS minimal medium (Teknova, Inc., Hollister, Calif., USA) containing 1 g/L glucose, 100 μmol/L thiamine (Sigma-Aldrich), and 100 mg/L kanamycin.

In each experiment, single colonies of E. coli FB21591 were transferred from LB plates, containing 2% (wt/vol) agar and 100 μg/L of kanamycin, to 5 mL of sterile LB medium (containing 8 g/L glucose, 100 μg/L kanamycin, and 0.1 mol/L MES) in test tubes. These cultures were then incubated on a roller drum at 60 rpm and 37° C. When the culture reached an OD₆₀₀ₙₘ of 1 (Spectronic 20 Genesys, Spectronic Instruments, Leeds, UK), 1.5 mL of culture medium was transferred from test tubes to 30 mL of LB or MOPS medium in a 250 mL baffled shake flask. The shake flask was then incubated at 37° C. on a shaker operating at 150˜220 rpm. The culture medium in the shake flask was used to inoculate the microbioreactor.

For microbioreactor experiments, DO, pH, and OD data were obtained on-line every 20 minutes after inoculation. Following each continuous culture experiment, the entire volume of the culture (˜150 μL) was harvested and the final OD₆₀₀ and pH values were measured. Calibration curves for OD readings were obtained by filling the microbioreactor with culture fluids with different biomass concentration. The OD₆₀₀ reading of the inoculum and the final OD₆₀₀ reading were then used to calibrate real-time OD measurement. Since the optical absorbance of PDMS changes after being dipped in water (Chang et al., 2003), the microbioreactor was filled with sterile water for more than 6 hours before each experiment to eliminate any potential changes in OD.

After each experiment, medium in the feeding tubing was collected and added to test tubes containing 5 mL of sterile LB medium as a contamination test. Turbidity of test tubes after incubation at 37° C. was used to detect possible cell back-growth during continuous fermentation in the microbioreactor. Images for PMMA and PDMS surfaces in the microbioreactor chamber were taken under microscopy. Safranin (Sigma-Aldrich) was used to stain E. coli cells on PMMA surface and to improve contrast with background.

Results and Discussion

Steady State Cell Culture

A critical requirement for chemostat experiments is the ability to achieve and sustain steady state conditions. We performed continuous culture experiments with E. coli, starting with an inoculum of metabolically active cells in MOPS medium. After inoculation, cells utilized all available oxygen and DO level dropped to zero rapidly in few hours. Correspondingly, the pH level of the culture broth decreased as a result of acetic acid byproduct formation due to fermentation (Han et al., 1992), and then recovered as the DO level recovers after 17 hours in the experiment. After about 60 hours DO, pH, and OD reached stable levels and steady state conditions in the microchemostat were established.

The net increase rate of bacterial biomass in suspension X is given by the simple balance Equation 1 (Herbert et al., 1956) for mixed bioreactors. The kinetics model of Equation 2 for continuous culture is the result of the dynamic balance between the carbon source-limited cell growth rates with medium feeding rates at steady states. With a medium feeding rate F of 1 μL/min and a reactor volume V of 150 μL, the specific cell growth rate μ, which equals the dilution rate D, is 0.4 hr⁻¹. This relatively low growth rate is characterized by DO level as high as ˜81%, and the steady state is maintained for ˜8 turnover times. OD₆₀₀ level stabilized at ˜1.05, and pH level is 6.5.

$$\begin{matrix}\frac{ⅆ X}{ⅆ t} = μ X - DX & ( 1 ) \\ μ = D = \frac{F}{V} & ( 2 )\end{matrix}$$

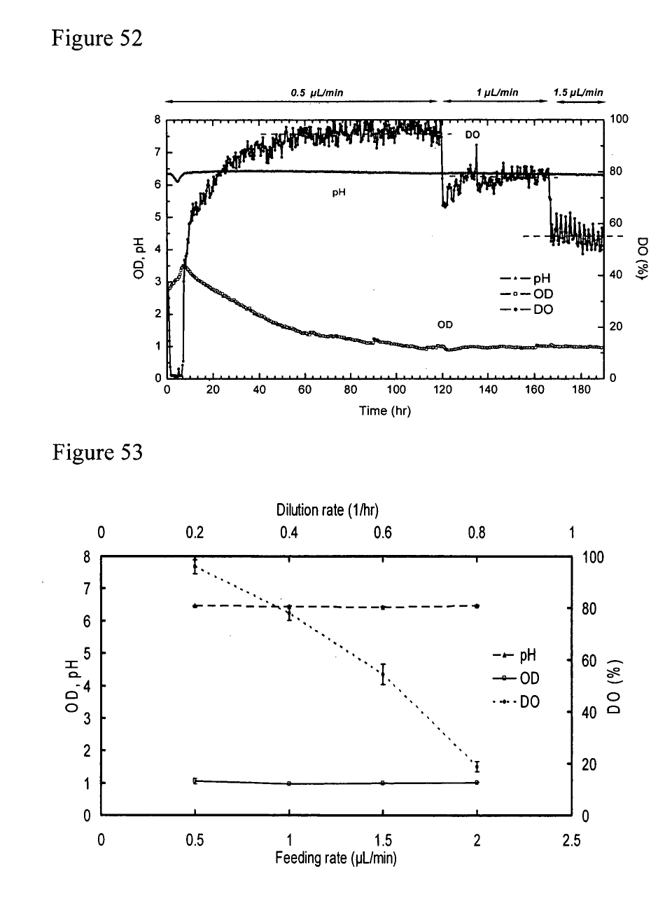

In a subsequent experiment multiple steady states are reached when medium feeding rate are increased from 0.5 μL/min to 1 μL/min and to 1.5 μL/min, sequentially (FIG. 52). Steady state conditions were maintained for at least 8 turnovers at each dilution rate. Steady DO levels observed were about 94%, 77%, and 56%, respectively. Lower DO levels at higher dilution rates are direct indications of faster growth and metabolism rates. Aerobic metabolism in the microchemostat makes pH level in the culture medium relatively stable at different dilution rates due to sufficient oxidative catabolism and the pH buffer capacity from phosphates in the MOPS medium. As the measurement for biomass concentration, OD₆₀₀ level also remained at a stable level of ˜1 (biomass concentration of ˜0.46 g cell dry weight/L), despite the changes of different dilution rates; this is consistent to bioprocess stoichoimetry observed in conventional bioreactors when glucose is the sole carbon and energy source for E. coli aerobic cultivation (Harvey, 1970; Shuler and Kargi, 2001). The robustness of the microchemostat is also demonstrated: for the steady state established at the 1 μL/min medium feeding rate in FIG. 52, DO, pH, and OD levels are very close to the values obtained in the earlier experiment.

Different cell culture conditions represented by DO, pH, and OD, are summarized in FIG. 52. This demonstrates the capability of microchemostat for effective maneuvering cell growth rate and culture environments.

Inhibition of Cell Back Growth and Wall Growth

Liquid medium upstream to the heated zone (i.e., between the medium source and the heated zone) was collected and incubated in fresh LB medium, and no cell growth was observed. In contrast cells were present upstream of the culture chamber in the un-heated feeding channel. With the implementation of local heating of the medium inflow channel, chemotaxis and back growth of E. coli cells were effectively inhibited.

### Example 13

### Construction of a Multilayer Microbioreactor with Integrated Fluidic and Optical Plugs

A microbioreactor system with microfluidic and optical connectors and integrated microlenses was constructed. The microbioreactor consists of five thermally-bonded poly(methylmethacrylate) (PMMA) layers as shown schematically in FIG. 54A. Precise thermal bonding of PMMA with different glass transition (TG) temperatures was done in two steps: three bottom layers were first bonded together and then bonded with top two layers. In the center of the system, a round reactor chamber was fabricated with a built-in magnetic spin bar mixer for mixing of fermentation medium. On the top side of the system four reversible “plug-n-pump” (Perozziello, 2004) microfluidic interconnections attach external tubing to three microchannels that lead to the reactor chamber, and serve for inoculation, reagent-feeding, sampling (from a sample reservoir), and waste outlet (FIG. 54B). Aseptic self-sealing of these interconnects was realized with custom-made O-rings (silastic elastomer, Dow Corning) placed in the upper PMMA layers.

A thin layer of spin-coated poly(dimethylsiloxane) (PDMS) covers the reactor chamber and serves as an aeration membrane. This thin PDMS layer is held by a thicker PDMS layer to facilitate device assembly, and covered by a grid structure to prevent bulging. A PMMA cork with a slightly larger diameter than the PMMA housing frame presses down on the PDMS and the silastic O-ring for sealing by friction. It also aligns an optical fiber for transmission measurement. Two recesses at the bottom of the bioreactor chamber accommodate pH and DO fluorescence lifetime sensors. Recesses beneath these sensors in the bottom PMMA layer accommodate and passively self-align optical connectors. In these connectors, optical fibers are held and align to spherical PDMS microlenses (FIG. 54C), thus connecting the microbioreactor system to external instruments. The assembled and bonded microbioreactor is shown in FIG. 54D.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. Furthermore, where the claims recite an apparatus, it is to be understood that methods of using the apparatus as described in any of the claims reciting methods are also disclosed, unless it would be evident to one of ordinary skill in the art that a contradiction or inconsistency would arise. Where the application or claims disclose one or more first structure(s) located within or passing through one or more second structure(s) or layer(s), it is to be understood that embodiments in which all or part of one or more of the first structure(s) is located within or passes through all or part of one or more of the second structure(s) or layer(s) are also disclosed. It is also to be understood that where the claims recite an apparatus that has particular features or characteristics, the invention encompasses an apparatus comprising means for implementing such features or characteristics. In addition, it is to be understood that any particular embodiment of the present invention that falls within the prior art may be explicitly excluded from the claims. Since such embodiments are deemed to be known to one of ordinary skill in the art, they may be excluded even if the exclusion is not set forth explicitly herein.

### REFERENCES

1. D. M. Disley, P. R. Morrill, K. Sproule, C. R. Lowe, “An optical biosensor for monitoring recombinant proteins in process media,” 14, 481-493 (1999).
2. D. E. Cane, C. T. Walsh, C. Khosla, “Harnessing the biosynthetic code: combinations, permutations, and mutations,” Science 282, 63-68 (1998).
3. M. Kleerebezemab, P. Hols, J. Hugenholtz, “Lactic acid bacteria as a cell factory: rerouting of carbon metabolism in Lactococcus lactis by metabolic engineering,” Enzyme Microb Technol. 26, 840-848 (2000).
4. D. C. Cameron, N. E. Altaras, M. L. Hoffman, A. J. Shaw, “Metabolic engineering of propanediol pathways,” Biotechnol Prog. 14, 116-25 (1998).
5. D. H. Pieper, W. Reineke, “Engineering bacteria for bioremediation,” Curr. Opin. Biotechnol. 11 262-70 (2000).
6. J. Ohlrogge, “Plant metabolic engineering: are we ready for phase two?,” Curr. Opin. Plant Biol. 2, 121-122.
7. S. Guillouet, A. A. Rodal, G.-H. An, P. A. Lessard, and A. J. Sinskey “Expression of the Escherichia coli catabolic threonine dehydratase, in Corynebacterium glutamicum and its effect on isoleucine production,” Applied and Environmental Microbiology 6, 3100-3107 (1999).
8. M. W Losey, M. A., Schmidt, and K. F. Jensen, “A micro packed-bed reactor for chemical synthesis”, In Microreaction Technology: Industrial Prospects (Ed, Ehrfeld, W.) Springer, Berlin, pp. 277-286 (2000).
9. R. Srinivasan, I.-M. Hsing, P. E. Berger, K. F. Jensen, S. L Firebaugh, M. A. Schmidt, M. P Harold, J. J. Lerou, and J. F Ryley. “Micromachined reactors for catalytic partial oxidation reactions”. AIChE Journal, 43, 3059-3069 (1997).
10. T. M Floyd, M. W. Losey, S. L. Firebaugh, K. F. Jensen and M. A Schmidt. “Novel liquid phase microreactors for safe production of hazardous specialty chemicals,” In Microreaction Technology: Industrial Prospects (Ed. Ehrfeld, W.) Springer, Berlin, pp. 171-180 (2000).
11. S. L Firebaugh, K. F. Jensen and M. A. Schmidt, “Miniaturization and integration of photoacoustic detection with a microfabricated chemical reactor system,” In Micro Total Analysis Systems 2000 (Eds, Berg, A. v. d., Olthuis, W. and Berveld, P.) Kluwer Academic Publishers, Dordrecht, pp. 49-52 (2000).
12. M. W Losey, M. A., Schmidt, and K. F. Jensen, “Microfabricated multiphase packed-bed reactors: characterization of mass transfer and reactions,” I&EC Research submitted (2000).
13. J. J Jackmann, T. Floyd, M. A. Schmidt, and K. F. Jensen, “Development of methods for on-line chemical detection with liquid-phase microchemical reactors using conventional and unconventional techniques,” In Micrototal Analysis Systems 2000 (Eds, Berg, A. v. d., Olthuis, W. and Bergveld, P.) Kluwer Academic Publishers, Dordrecht, pp. 155-159 (2000).
14. S. W. Lee, P. E. Laibinis, “Protein resistant coatings for glass and metal oxide surfaces derived from oligo(ethylene glycol)-terminated alkyltrichlorosilanes.” Biomaterials 19, 1669-1675 (1998).
16. N. Y. Kim, P. E. Laibinis, “Covalent modification of hydrogen-terminated silicon surfaces,” In New Directions in Materials Synthesis, C. H. Winter, Ed.; ACS Symposium Series, 727, 157-168 (1999).
17. Y. K. Namyong, N. L. Jeon, I. S. Choi, S. Takami, Y. Harada, K. R, Finnie, G. S. Girolami, R. G. Nuzzo, G. M. Whitesides, and P. E. Laibinis, “Surface-initiated ring-opening metathesis polymerization on silicon,” Macromolecules, 33, 2793-2795 (2000).
18. J. J. Hickman, D. Ofer, C. Zou, M. S. Wrighton, P. E. Laibinis, G. M. Whitesides, “Selective functionalization of gold microstructures with ferrocenyl derivatives via reaction with thiols or disulfides: Characterization by electro-chemistry and auger electron spectroscopy,” J. Am. Chem. Soc., 113, 1128-1132 (1991).
19. J. J. Hickman, D. Ofer, M. S. Wrighton, P. E. Laibinis, G. M. Whitesides, “Molecular self-assembly of two-terminal, voltammetric microsensors with an internal reference,” Science, 252, 688-691 (1991).
20. R. Michalitsch; P. E. Laibinis, “Electrochemical halide detection by gold electrodes exposing a layer of silver atoms,” Angewandte Chemie (submitted).
21. Q.-Y. Li, L. M. Davis, “Rapid and efficient detection of single chromophore molecules in aqueous solution,” Applied Optics, 34, 3208-3217 (1995).
22. A. G. Mignani, F. Baldini, “In-vivo biomedical monitoring by fiber-optic systems,” J. of Lightwave Tech., 13, 1396-1406 (1995).
23. l.-Y. Li, L. M. Davis, Applied Optics, 34, 3208-3217 (1995)
24. C.-M. Chun, W. Lo, K.-Y. Wong, “Application of a luminescence-based pH optrode to monitoring of fermentation by Klebsiella pneumoniae,” Biosensors and Bioactuators, 15, 7-11 (2000).
25. M. E. Lippitsch, S. Draxler, D. Kieslinger, Sensors and Actuators B, 38-39, 96-102 (1997).
26. S. Draxler, M. E. Lippitsch, “pH sensors using fluorescence decay time,” Sensors and Acutators B, 29, 199-203 (1995).
27. B. J. Feilmeier, G. Iseminger, D. Schroeder, H. Webber, G. J. Phillips, “Green fluorescent protein functions as a reporter for protein localization in escherichia coli,” J. Bacteriol., 182, 4068-4076 (2000).
28. D. H. Edwards, H. B. Thomaides, J. Errington “Promiscuous targeting of bacillus subtilis cell division protein DivIVA to division sites in Escherichia coli and fission yeast,” Embo. J, 19, 2719-27 (2000).
29. D. Aldon, B. Brito, C. Boucher, S. Genin, “A bacterial sensor of plant cell contact controls the transcriptional induction of Ralstonia solanacearum pathogenicity genes,” Embo J 19, 2304-14 (2000).
30. K. R. Finer, J. J. Finer “Use of Agrobacterium expressing green fluorescent protein to evaluate colonization of sonication-assisted Agrobacterium-mediated transformation-treated soybean cotyledons” Lett Appl Microbiol. 30, 406-410 (2000).
31. M. F. Garcia-Parajo, G. M. Segers-Nolten, J. Veerman, J. Greve, N. F. van Hulst “Real-time light-driven dynamics of the fluorescence emission in single green fluorescent protein molecules,” Proc Natl Acad Sci U.S.A., 97, 7237-7242 (2000).
32. A. K. Dunn, J. Handelsman, “A vector for promoter trapping in Bacillus cereus,” Gene, 226, 297-305 (1999).
33. J. N. Demas and B. A. DeGraff, “Design and Applications of Highly Luminescent Transition Metal Complexes”, Analytical Chemistry, vol. 63, ppl 829-837, 1991.
34. S. B. Bamobt, et al., “Phase Fluorometric Sterilizable Optical Oxygen Sensor”, Biotechnology and Bioengineering, vol. 43, ppl 1139-1145, 1994.
35. A. K. McEvoy, et al., “Dissolved oxygen sensor based on fluorescence quenching of oxygen-sensitive ruthenium complexes immobilized in sol-gel derived porous silicon coatings”, Analyst, vol. 121, pp. 785-788, 1996.
36. Z. Zhujun, et al., “A Carbon Dioxide sensor Based on Fluorescence”, Analytica Chimica Acta, 160: 305-309 (1984).
37. Y. Kawabata, et al., “Fiber-Optic Sensor for Carbon Dioxide with a pH Indicator Dispersed in a Poly (Ethylene Glycol) Membrane”, Analytica Chimica Acta, 219:223-229 (1989).
38. B. Weigl, et al., “Chemically and mechanically resistant carbon dioxide optrode based on a covalently immobilized pH indicator”, Analytica Chimica Acta, 282:335-343 (1993).
39. Y. Kostov, et al., “Low-cost Microbioreactor for High Throughput Bioprocessing”, Biotechnology and Bioengineering, vol. 72, pp. 346-352, 2001.
40. S. DeWitt, “Microreactors for Chemical Synthesis”, Curr. Op. in Biotechnology, 3:350-356, 1999.
41. F. Neidhardt, (ed.), “Escherichia Coli and Salmonella: Cellular and Molecular Biology, CD-ROM edition, September 1999, Amer Society for Microbiology; ISBN: 1555811647.
42. E. Winzeler, “Functional characterization of the S. cerevisiae genome by gene deletion and parallel analysis”, Science 1999 Aug. 6;285(5429):901-6.
43. J. Sambrook, et al., Molecular Cloning—A Laboratory Manual, 2^{nd} ed.: Cold Spring Harbor Laboratory Press, 1989.
44. T. C. Merkel, et al., “Gas sorption, diffusion, and permeation in poly(dimethylsiloxane)”, J. of Polymer Science, Part B—Polymer Physics, vol. 38, ppl 415-434, 2000.
45. Perry's Chemical Engineering Handbook: R.R. Donnelley & Sons Company, 1984.
46. B. Atkinson and F. Mavituva, Biochemical Engineering and Biotechnology Handbook. New York: The Nature Press, 1983.
47. Rooney, M. T. V. and W. R. Seitz, An optically sensitive membrane for pH based on swellable polymer microspheres in a hydrogel. Analytical Communications, 1999. 36(7): p. 267-270.
48. Vacik, J., et al., The Effect of Ph and Temperature on the Electrical-Conductivity of Membranes Made of Methacrylic-Acid Co-Polymers. Collection of Czechoslovak Chemical Communications, 1983. 48(11): p. 3071-3078.
49. Vacik, J. and J. Kopecek, Specific Resistances of Hydrophilic Membranes Containing Ionogenic Groups. Journal of Applied Polymer Science, 1975. 19(11): p. 3029-3044.
50. Sheppard, N. F., R. C. Tucker, and S. Salehihad, Design of a Conductimetric Ph Microsensor Based on Reversibly Swelling Hydrogels. Sensors and Actuators B-Chemical, 1993. 10(2): p. 73-77.
51. Sheppard, N. F., M. J. Lesho, and P. McNally, Microfabricated Conductimetric Ph Sensor. Sensors and Actuators B-Chemical, 1995. 28(2): p. 95-102.
52. Zabriskie, D, et al. “Estimation of fermentation biomass concentration by measuring culture fluorescence”, Appl. Eur. Microbiol. 1978, Vol. 35(2), pp. 337-343;
53. Marose, S., et al. “Two-dimensional fluorescence spectroscopy: A new tool for online bioprocess monitoring”, Biotechnology Progress, 1998, 14, pp. 63-74).
54. Stephanopolous, G., ed. Bioprocessing. Second ed. Biotechnology, ed. H.-J. Rehm, et al. Vol. 3. 1993, VCH Publishers Inc.: New York.
55. Bailey, J. E. and D. F. Ollis, Biochemical Engineering Fundamentals. Second ed. McGraw-Hill chemical engineering series. 1986: McGraw-Hill, Inc.
56. Demas, J., et al., “Applications of luminescent transition platinum group metal complexes to sensor technology and molecular probes”, Coordination Chemistry Reviews, 211, 2001, pp. 317-351.
57. Demas, J., et al. “Oxygen sensors based on luminescence quenching”, Analytical Chemistry News and Features, Dec. 1, 1999, pp. 793A-800A.
58. Duffy, D., et al., “Rapid prototyping of microfluidic systems in poly(dimethylsiloxane),” Analytical Chemistry, vol. 70, pp. 4974-4984, 1998.
59. Mulder, M., Basic Principles of Membrane Technology. Second ed. 1996: Kluwer Academic Publishers.
60. Tamai, Y., H. Tanaka, and K. Nakanishi, Molecular Simulation of Permeation of Small Penetrants through Membranes .1. Diffusion-Coefficients. Macromolecules, 1994. 27(16): p. 4498-4508.
61. Tamai, Y., H. Tanaka, and K. Nakanishi, Molecular Simulation of Permeation of Small Penetrants through Membranes .2. Solubilities. Macromolecules, 1995. 28(7): p. 2544-2554.
62. Watson, J. M. and M. G. Baron, The behaviour of water in poly(dimethylsiloxane). Journal of Membrane Science, 1996. 110(1): p. 47-57.
63. Fritz, L. and D. Hofmann, Molecular dynamics simulations of the transport of water-ethanol mixtures through polydimethylsiloxane membranes. Polymer, 1997. 38(5): p. 1035-1045.
64. Merkel, T. C., et al., Gas sorption, diffusion, and permeation in poly(dimethylsiloxane) Journal of Polymer Science Part B—Polymer Physics, 2000. 38(3): p. 415-434.
65. Stern, S. A., V. M. Shah, and B. J. Hardy, Structure-Permeability Relationships in Silicone Polymers. Journal of Polymer Science Part B—Polymer Physics, 1987. 25(6): p. 1263-1298.
66. Pinnau, I. and L. G. Toy, Gas and vapor transport properties of amorphous perfluorinated copolymer membranes based on 2,2-bistrifluoromethyl-4,5-difluoro-1,3-dioxole/tetrafluoroethylene. Journal of Membrane Science, 1996. 109(1): p. 125-133.
67. C. McDonagh, et al., “Tailoring of sol-gel films for optical sensing of oxygen in gas and aqueous phase”, Analytical Chemistry, Vol. 70(1), Jan. 1, 1998, pp. 45-50.
68. S. Parekh, et al., “Improvement of microbial strains and fermentation processes”, Appl. Microbiol. Technol., 54:287-301, 2000.
69. A. L. Demain and J. E. Davis, Manual of industrial microbiology and technology, 2^{nd} ed., Am. Soc. Microbiol. Press, Washington, D.C., 1998.
70. Swarz, J. R., “Advances in Escherichia coli production of therapeutic proteins”, Current Opinion in Biotechnology, 2001. 12: p. 195-201.
71 Baneyx, F., “Recombinant protein expression in Escherichia coli”, Current Opinion in Biotechnology, 1999. 10(5): p. 411-421.
72. Cereghino G P, Cregg J M., “Applications of yeast in biotechnology: protein production and genetic analysis”, Curr Opin Biotechnol. 1999 October;10(5):422-7.
73. S. Hashimoto and A. Ozaki, “Whole microbial cell processes for manufacturing amino acids, vitamins or ribonucleotides”, Curr Opin Biotechnol. 1999 December;10(6):604-8.
74. Tanaka, H., J. Takahashi, and K. Ueda, “Studies on Effect of Agitation on Mycelia in Submerged Mold Culture .3. Standard for Intensity of Agitation Shock on Mycelia on Agitation of Mycelial Suspensions”, Journal of Fermentation Technology, 1975. 53(1): p. 18-26.
75. Oldshue, Y., “Fermentation mixing scale-up techniques”, Biotechnology and Bioengineering, 1966. 8: p. 3-24.
76. Hauser, H., et al. (eds.), Mammalian Cell Biotechnology in Protein Production, W de Gruyter, 1997.
77. Campbell, S., The Science and Engineering of Microelectronic Fabrication, 2nd Ed., Oxford: 2001.
78. Madou, M., Fundamentals of Microfabrication, Boca Raton: CRC Press, 1997.
79. Lahann, J., “Reactive Polymer Coatings: A Platform for Patterning Proteins and Mammalian Cells onto a Broad Range of Materials”, Langmuir, May 2002.
80. Ferraro, J. R., et al., Introductory Raman Spectroscopy, Academic Press; 2nd edition (Oct. 28, 2002).
81. Laserna, J. J. (Ed.) Modern Techniques in Raman Spectroscopy, John Wiley & Son Ltd; 1 edition (Aug. 28, 1996).
82. Pramanik, J. & Keasling, J. D. Stoichiometric model of Escherichia coli metabolism: incorporation of growth-rate dependent biomass composition and mechanistic energy requirements. Biotechnology and Bioengineering 56, 398-421, 1997.
83. Wolfbeis, O. S., “Fiber-Optic Chemical Sensors and Biosensors”, Anal. Chem. 74: 2663-2678, 2002.
84. Anderlei T, Buchs J. 2001. Device for sterile online measurement of the oxygen transfer rate in shaking flasks. Biochemical Engineering Journal 7(2):157-162.
85. Ausubel F M, Brent R, Kingston R E, Moore D D, Seidman J G, Smith J A, Struhl K, editors. 1995. Short Protocols in Molecular Biology. Third ed: John Wiley & Sons, Inc.
86. Bacon J R, Demas J N. 1987. Determination of oxygen concentrations by luminescence quenching of a polymer-immobilized transition-metal complex. Analytical Chemistry 59(23):2780-5.
87. Bauer S, Shiloach J. 1974. Maximal exponential growth rate and yield of E. coli obtainable in a bench-scale fermentor. Biotechnology and Bioengineering 16(7):933-41.
88. Brunker R L, Brown O R. 1971. Effects of hyperoxia on oxidized and reduced NAD and NADP concentrations in Escherichia coli. Microbios 4(15):193-203.
89. Carraway E R, Demas J N, DeGraff B A, Bacon J R. 1991. Photophysics and photochemistry of oxygen sensors based on luminescent transition-metal complexes. Analytical Chemistry 63(4):337-42.
90. Chartrain M, Salmon P M, Robinson D K, Buckland B C. 2000. Metabolic engineering and directed evolution for the production of pharmaceuticals. Current Opinion in Biotechnology 11(2):209-214.
91. Demas J N, DeGraff B A. 1991. Design and applications of highly luminescent transition metal complexes. Analytical Chemistry 63(17):829-837.
92. Demas J N, DeGraff B A, Coleman P B. 1999. Oxygen sensors based on luminescence quenching. Analytical Chemistry 71(23):793A-800A.
93. Demas J N, DeGraff B A, Xu W. 1995. Modeling of luminescence quenching-based sensors: comparison of multisite and nonlinear gas solubility models. Analytical Chemistry 67(8):1377-80.
94. Ge X, Kostov Y, Rao G. 2003. High-stability non-invasive autoclavable naked optical CO2 sensor. Biosensors & Bioelectronics 18(7):857-865.
95. Gottlieb S F. 1971. Effect of hyperbaric oxygen on microorganisms. Annual Review of Microbiology 25:111-152.
96. Gram A. 1997. Biochemical engineering and industry. Journal of Biotechnology 59(1-2):19-23.
97. Gupta A, Rao G. 2003. A study of oxygen transfer in shake flasks using a non-invasive oxygen sensor. Biotechnology and Bioengineering 84(3):351-358.
98. Hermann R, Lehmann M, Buchs J. 2003. Characterization of gas-liquid mass transfer phenomena in microtiter plates. Biotechnology and Bioengineering 81(2): 178-186.
99. John G T, Goelling D, Klimant I, Schneider H, Heinzle E. 2003a. pH-Sensing 96-well microtitre plates for the characterization of acid production by dairy starter cultures. Journal of Dairy Research 70(3):327-333.
100. John G T, Klimant I, Wittmann C, Heinzle E. 2003b. Integrated optical sensing of dissolved oxygen in microtiter plates: A novel tool for microbial cultivation. Biotechnology and Bioengineering 81(7):829-836.
101. Kim J W, Lee Y H. 1998. Development of microfermenter chip. Journal of the Korean Physical Society 33:S462-S466.
102. Klimant I, Wolfbeis O S. 1995. Oxygen-sensitive luminescent materials based on silicone-soluble ruthenium diimine complexes. Analytical Chemistry 67(18):3160-6.
103. Konz J O, King J, Cooney C L. 1998. Effects of oxygen on recombinant protein expression. Biotechnology Progress 14(3):393-409.
104. Kosch U, Klimant I, Werner T, Wolfbeis O S. 1998. Strategies to design pH optodes with luminescence decay times in the microsecond time regime. Analytical Chemistry 70(18):3892-3897.
105. Kostov Y, Harms P, Randers-Eichhorn L, Rao G. 2001. Low-cost microbioreactor for high-throughput bioprocessing. Biotechnology and Bioengineering 72(3):346-352.
106. Lakowicz J R, editor. 1999. Principles of Fluorescence Spectroscopy. 2nd edition ed: Plenum Publishing Corporation.
107. Lamping S R, Zhang H, Allen B, Shamlou P A. 2003. Design of a prototype miniature bioreactor for high throughput automated bioprocessing. Chemical Engineering Science 58(3-6):747-758.
108. Lide D R, editor. 2001. CRC Handbook of Chemistry and Physics. 82nd ed: CRC Press.
109. Liebsch G, Klimant I, Krause C, Wolfbeis O S. 2001. Fluorescent imaging of pH with optical sensors using time domain dual lifetime referencing. Analytical Chemistry 73(17):4354-4363.
110. Lin J. 2000. Recent development and applications of optical and fiber-optic pH sensors. Trac-Trends in Analytical Chemistry 19(9):541-552.
111. Maharbiz M M, Holtz W J, Howe R T, Keasling J D. 2004. Microbioreactor arrays with parametric control for high-throughput experimentation. Biotechnology and bioengineering 85(4):376-81.
112. Maharbiz M M, Holtz W J, Sharifzadeh S, Keasling J D, Howe R T. 2003. A microfabricated electrochemical oxygen generator for high-density cell culture arrays. Journal of Microelectromechanical Systems 12(5):590-599.
113. Maier U, Buchs J. 2001. Characterisation of the gas-liquid mass transfer in shaking bioreactors. Biochemical Engineering Journal 7(2):99-106.
114. Merkel T C, Bondar V I, Nagai K, Freeman B D, Pinnau I. 2000. Gas sorption, diffusion, and permeation in poly(dimethylsiloxane). Journal of Polymer Science Part B-Polymer Physics 38(3):415-434.
115. Monod J. 1949. The growth of bacterial cultures. Annual Review of Microbiology 3:371-394.
116. Onken U, Liefke E. 1989. Effect of total and partial pressure (oxygen and carbon dioxide) on aerobic microbial processes. Advances in Biochemical Engineering/Biotechnology 40(Bioprocesses Eng.): 137-69.
117. Parekh S, Vinci V A, Strobel R J. 2000. Improvement of microbial strains and fermentation processes. Applied Microbiology and Biotechnology 54(3):287-301.
118. Perry R H, Green D, editors. 1984. Perry's Chemical Engineering Handbook: R.R. Donnelley & Sons Company.
119. Randers-Eichhorn L, Bartlett R A, Frey D D, Rao G. 1996. Noninvasive oxygen measurements and mass transfer considerations in tissue culture flasks. Biotechnology and Bioengineering 51(4):466-478.
120. Schmid R D, Hammelehle R. 2003. Pocket Guide to Biotechnology and Genetic Engineering: Wiley-VCH.
121. Shanks J V, Stephanopoulos G. 2000. Biochemical engineering—bridging the gap between gene and product. Current Opinion in Biotechnology 11: 169-170.
122. Stitt D T, Nagar M S, Haq T A, Timmins M R. 2002. Determination of growth rate of microorganisms in broth from oxygen-sensitive fluorescence plate reader measurements. BioTechniques 32(3):684, 686, 688-689.
123. Tolosa L, Kostov Y, Harms P, Rao G. 2002. Noninvasive measurement of dissolved oxygen in shake flasks. Biotechnology and Bioengineering 80(5):594-597.
124. Van Suijdam J C, Kossen N W F, Joha A C. 1978. Model for oxygen transfer in a shake flask. Biotechnology and Bioengineering 20(11):1695-709.
125. Verhulst P-F. 1838. Notice sur la loi que la population suit dans son accroissement. Correspondance Mathematique et Physique (10): 113-121.
126. Weuster-Botz D, Altenbach-Rehm J, Arnold M. 2001. Parallel substrate feeding and pH-control in shaking-flasks. Biochemical Engineering Journal 7(2):163-170.
127. Wittmann C, Kim H M, John G, Heinzle E. 2003. Characterization and application of an optical sensor for quantification of dissolved O₂ in shake-flasks. Biotechnology Letters 25(5):377-380.
128. Moeser, G. D., et al., 2002. Water-based magnetic fluids as extractants for synthetic organic compounds, Ind. Eng. Chem. Res., 41, 4739-4749.
129. Nihonyanagi, S., Ye S., Uosaki, K. 2001. Electrochimica Acta, 46, 3057-3061.
130. Hyun-Goo Choi, H-G., Zhang, Z., Boccazzi P., Laibinis, P. E., Sinskey, A. J., Jensen, K. F. Poly(Ethylene Glycol) (PEG)-Modified Poly(Dimethylsiloxane) (PDMS) for Protein- and Cell-Resistant Surfaces in Microbioreactor, Proceedings of mTAS 2003-Seventh International Conference on Miniaturized Chemical and Biochemical Analysis Systems, M. A. Northrup, K. F. Jensen, and D. J. Harrison (Eds.), Squaw Valley, Calif., Transducers Research Foundation, 2003 pp. 1105-8.
131. Stephanopoulos, G. N., Aristidou, A. A. and Nielsen, J. 1998. Metabolic Engineering. San Diego: Academic Press.
132. Nielsen, J. 1998. Metabolic engineering: Techniques for analysis of targets for genetic manipulations. Biotechnology and Bioengineering 58(2-3):125-132.
133. Sydor, J. R. and Nock, S. 2003. Protein expression profiling arrays: tools for the multiplexed high-throughput analysis of proteins. Proteome Science, 1:3 (published on the Internet at www.Proteomsci.com/content/1/1/3.
133. Walther, I., B. H. v. d. Schoot, et al. (1994). “Development of a miniature bioreactor for continuous culture in a space laboratory.” Journal of Biotechnology 30(1): 21-32.
134. Walther, I., B. v. d. Schoot, et al. (1999). “Microtechnology in Space Bioreactors.” Chimia 53(75-80).
135. Walther, I., B. v. d. Schoot, et al. (2000). “Performace of a Miniaturized Bioreactor in Space Flight: Microtechnology at the Service of Space Biology.” Enzyme and Microbial Technology 27: 778-783.
136. Dykhuizen, D. E. and Dean, Antony. 2004. Evolution of specialists in an experimental microcosm. Genetics 167: 2015-2026.
137. Z. Zhang, N. Szita, P. Boccazzi, A. J. Sinskey, and K. F. Jensen, “Monitoring and control of cell growth in fed-batch microbioreactors,” μTAS2003, 765(2003).
138. A. Zanzotto, N. Szita, P. Boccazzi, P. Lessard, A. J. Sinskey, and K. F. Jensen, “A Membrane-aerated Microbioreactor for High-throughput Bioprocessing,” Biotech. Bioeng., 85, 376(2004).
139. Z. Zhang, P. Boccazzi, H.-G. Choi, N. Szita, A. J. Sinskey, K. F. Jensen, “A microchemostat—continuous cell culture in microbioreactors,” μTAS2004, 231(2004).
140. N. Szita, P. Boccazzi, Z. Zhang, A. Zanzotto, A. J. Sinskey, K. F. Jensen, “Multiplexedmicrobioreactor system for high-throughput bioprocess,” μTAS2004, 390(2004).
141. G. Perozziello, M. F. Jensen, J. E. Mc Cormack, F. Bundgaard, and O. Geschke, “Plug'n'pump fluidic interconnection,” μTAS2004, 575(2004).
142. K. Maeda, Y. Imae, J. Shioi, F. Oosawa, Journal of Bacteriology, 1976, 127, 1039-1046.
143. J. Adler, Scientific American. 1976, 234, 40-47.
144. W.-J. Chang, D. Akin, M. Sedlak, M. R. Ladisch, R. Bashir, Biomedical Microdevices, 2003, 5, 281-290.
145. M. F. Jensen, U. Kruehne, L. H. Christensen, O. Geschke Micromech. and Microeng. 15 (2005) 91-97.
146. Marc P. Christensen, Pedrag Miojkovic, Michael J. McFadden and Michael W. Haney IEEE Journal on selected topics in Quantum Electronics, Vol. 9, No. 2, p 548 March/April 2003.

1. A microbioreactor comprising comprising:
at least one culture vessel having an interior volume of less than 1 ml;
a mechanism for actively mixing the contents of the culture vessel;
an inflow port to allow fresh culture medium to be continuously supplied to the culture vessel; and
an outflow port to allow culture medium to be continuously removed from the culture vessel at the same rate as fresh medium is supplied, such that a constant fluid volume and constant growth conditions are maintained within the culture vessel for a prolonged period of time after cells cultured in the culture vessel reach a steady state.

2-137. (canceled)

138. The microbioreactor of claim 1, wherein the microbioreactor is capable of operating as a microchemostat.

139. The microbioreactor of claim 1, wherein the mechanism for actively mixing the contents of the culture vessel comprises a stirbar.

140. The microbioreactor of claim 1, wherein at least a portion of a wall of the culture vessel comprises a gas-permeable membrane.

141. The microbioreactor of claim 1, further comprising:
an internal sensor that detects or measures dissolved oxygen within the vessel; and
an internal sensor that detects or measures pH within the vessel.

142. The microbioreactor of claim 1, further comprising an inflow channel or tube in communication with the inflow port and an outflow channel or tube in communication with the outflow port.

143. The microbioreactor of claim 142, wherein the inflow channel flows through a portion of the microbioreactor that inhibits cell growth within and movement of living cells into or through at least a portion of the inflow channel.

144. The microbioreactor of claim 142, further comprising means for inhibiting cell growth within, and movement of living cells into or through, at least a portion of the inflow channel or tube.

145. The microbioreactor of claim 144, wherein the means comprises one or more items selected from the group consisting of: (i) a heating element, (ii) a device that emits electromagnetic radiation sufficient to kill cells, and (iii) a filter having a pore size selected to prevent passage of cells through the filter.

146. The microbioreactor of claim 142, further comprising:
a collection chamber in communication with the outflow channel; and
a second channel or tube in communication with the outflow channel.

147. The microbioreactor of claim 142, wherein the collection chamber is located in a portion of the microbioreactor that maintains conditions that inhibit metabolic activity of cells within the collection chamber.

148. The microbioreactor of claim 146, further comprising means for inhibiting metabolic activity of cells within the collection chamber.

149. The microbioreactor of claim 148, wherein the means comprises one or more items selected from the group consisting of: (i) a cooling element; (ii) a second channel or tube in communication with the collection chamber and a supply of a cytostatic or cytotoxic agent, so that the chamber is supplied with a cytotoxic or cytostatic agent that inhibits metabolic activity of cells within the collection chamber.

150. A culture system comprising the microbioreactor of claim 1, further comprising:
means for collecting an optical signal from the interior of the culture vessel.

151. The microbioreactor of claim 1, wherein the interior of the culture vessel comprises a well located at least in part within a first body layer of material.

152. The microbioreactor of claim 151, wherein the well is located entirely within the first body layer of material.

153. The microbioreactor of claim 151, wherein the inflow port and outflow port are in communication with channels located at least in part within the first body layer.

154. The microbioreactor of claim 151, further comprising a second body layer having a void therein, wherein the first and second body layers are separated by a gas-permeable membrane and are positioned such that the void in the second body layer is separated from the well in the first body layer by the gas-permeable membrane, so that gas is exchanged between the interior of the culture vessel and the external environment.

155. The microbioreactor of claim 154, further comprising a layer of material covering the void, so that the void and the covering layer define an enclosed headspace located substantially opposite the interior of the culture vessel.

156. The microbioreactor of claim 151, wherein the first body layer is substantially made of a rigid material.

157. The microbioreactor of claim 151, further comprising:
a second body layer having a void therein; and
a third body layer having a void therein and located between the gas-permeable membrane and the second body layer, wherein both the second and third body layers are positioned such that the voids in the second and third body layers are located substantially opposite one another and separated from the well in the first body layer by the gas-permeable membrane, so that gas is exchanged between the interior of the culture vessel and the external environment.

158. The microbioreactor of claim 157, further comprising a layer of material covering the void in the second body layer, so that the void and the covering layer define an enclosed headspace located substantially opposite the interior of the culture vessel.

159. The microbioreactor of claim 157, wherein the first and second body layers are substantially made of a rigid substance.

160. The microbioreactor of claim 157, wherein the third body layer serves as a gasket for the gas-permeable membrane.

161. The microbioreactor of claim 157, further comprising a substrate layer that supports the first body layer.

162. The microbioreactor of claim 151, wherein the first body layer comprises a central section in which the well is located and one or more sections spaced apart from the central portion but physically connected thereto by one or more connecting elements.

163. The microbioreactor of claim 162, wherein at least one channel extends from the culture vessel through a connecting element and into an adjacent section.

164. The microbioreactor of claim 162, wherein at least one of the sections comprises a zone that inhibits cell movement, cell growth, or both, and through which a medium inflow channel passes.

165. The microbioreactor of claim 164, wherein the zone that inhibits cell movement, cell growth, or both is located within a section spaced apart from the section in which the well is located.

166. The microbioreactor of claim 164, wherein the zone is heated to a temperature sufficient to substantially inhibit bacterial chemotaxis.

167. The microbioreactor of claim 162, wherein a section that is spaced apart from the section in which the well is located comprises a zone that inhibits cell metabolism and that contains a sample collection chamber.

168. The microbioreactor of claim 167, wherein the zone is cooled to a temperature sufficiently low to a temperature low enough to substantially inhibit cell metabolism.

169. The microbioreactor of claim 1, wherein the microbioreactor optionally comprises one or more channels in communication with the culture vessel, and wherein at least a portion of an interior surface of the culture vessel, at least a portion of an interior surface of one or more of the channels, or both, is modified to resist adherence of cells, proteins, or both.

170. The microbioreactor of claim 169, wherein the modification comprises attachment of a polymer containing PEG to the surface.

171. The microbioreactor of claim 170, wherein the polymer is a polymer comprising a poly(acrylic acid) backbone with PEG-containing side chains grafted thereto.

172. The microbioreactor of claim 171, wherein the polymer is a PAA-g-(PEG-r-PPG) comb polymer.

173. A culture system comprising the microbioreactor of claim 1, further comprising a pumping system.

174. A culture system comprising the microbioreactor of claim 1, further comprising:
a medium reservoir in communication with the culture vessel and elevated above it so as to create pressure that drives medium into and out of the culture vessel.

175. A method of performing cell culture comprising:
introducing at least one cell into a microbioreactor that comprises a culture vessel having an interior volume of less than 1 ml;
continuously flowing fresh culture medium into the vessel while continuously removing culture medium containing cells from the culture vessel at the same rate as that with which fresh medium enters the vessel so that a constant medium volume is maintained in the culture vessel;
actively mixing the contents of the culture vessel;
maintaining the cells for sufficient time to achieve a first steady state.

176. The method of claim 175, further comprising:
maintaining the at least one cell under constant culture conditions for an additional period of time.

177. The method of claim 175, wherein the cells produce a product used in the pharmaceutical, food, and/or chemical industries.

178. The method of claim 177, wherein the product is selected from the group consisting of: primary and secondary metabolites, enzymes, recombinant proteins, and vaccine antigens.

179. The method of claim 175, further comprising altering the dissolved oxygen concentration in the medium in the culture vessel, altering the mass transfer coefficient (k_{L}a) of oxygen into the medium in the culture vessel, altering the composition of the medium entering the culture vessel, altering the pH of the contents of the culture vessel, or any combination of the foregoing.

180. The method of claim 175, further comprising measuring cell density, dissolved oxygen, pH, or any combination of the foregoing while culturing the at least one cell.

181. The method of claim 175, further comprising introducing medium into the culture vessel through an inflow channel or tube and inhibiting cell growth within, and movement of living cells into or through, at least a portion of the inflow channel or tube.

182. The method of claim 175, further comprising collecting cells in a collection chamber in communication with the culture vessel and inhibiting metabolic activity of cells within the collection chamber.

183. The method of claim 175, further comprising:
collecting one or more samples of the medium that is removed from the vessel in the continuous removal step; and
performing an analytical procedure on the sample or samples.

184. The method of claim 183, further comprising selecting a cell strain or bioprocess parameter based on the result of the analytical procedure.

185. The method of claim 175, further comprising:
altering the medium inflow and outflow rates, oxygenation rate, pH, composition, or any combination of the foregoing, so as to alter the growth conditions in the culture vessel; and
maintaining the culture for a time sufficient to reach a second steady state.

186. The method of claim 175, further comprising steps of:
collecting one or more samples of the medium that is removed from the vessel in the continuous removal step while the culture is in the first steady state;
collecting one or more samples of the medium that is removed from the vessel in the continuous removal step while the culture is in the second steady state; and
performing an analytical procedure on the sample or samples removed in the first and second steady states.

187. A method of selecting a strain that produces a desired product or degrades an unwanted compound comprising steps of:
culturing a plurality of different strains, each in a microbioreactor of claim 1;
measuring the amount of the desired or unwanted product in each of the microbioreactors; and
selecting a strain that produces an optimum amount of a desired product or degrades a maximum amount of the unwanted compound.

188. A method of selecting a bioprocess parameter comprising steps of:
culturing cells of an organism type in a plurality of individual microbioreactors of claim 1 under constant growth conditions, wherein the microbioreactors are operated under conditions in which the value of the bioprocess parameter varies between the individual microbioreactors and wherein the organism produces a product or degrades a compound;
monitoring biomass, product formation, or compound degradation in each of the microbioreactors; and
identifying the value of the bioprocess parameter that results in optimum biomass, optimum product formation, or optimum compound degradation.

189. The method of claim 188, in which the bioprocess parameter is actively controlled.

190. A method of performing a fermentation comprising:
culturing cells in a production scale fermentor, wherein one or more bioprocess parameters for the production scale fermentor is selected according to the method of claim 188.

191. A method of modifying a polymeric surface other than a PDMS surface so as to confer resistance to adherence of cells, proteins, or both, comprising steps of:
assembling an amine-terminated self assembled monolayer on the surface; and
contacting the surface with a PEG-containing polymer under conditions in which the PEG-containing polymer contains sufficient negative charges to cause adsorption to the self-assembled monolayer.

192. The method of claim 191, wherein the polymeric surface is PMMA or poly(carbonate).

193. The method of claim 191, wherein the PEG-containing polymer comprises a PAA or PMAA backbone.

194. The method of claim 191, further comprising the step of: generating free OH groups on the polymeric surface prior to assembling the amine-terminated self-assembled monolayer.

195. An apparatus comprising at least one polymeric surface modified according to the method of claim 191.
